(19) United States
(12) Patent Application Publication
(10) Pub. No.: US 2012/0101134 A1
(43) Pub. Date: Apr. 26, 2012

(54) 5-MEMBERED HETEROARYL DERIVATIVES USED AS SPHINGOSINE 1-PHOSPHATE RECEPTOR AGONISTS

(75) Inventors: Guanghui Deng, Shanghai (CN); Xichen Lin, Shanghai (CN); Qinghua Meng, Shanghai (CN); Feng Ren, Shanghai (CN); Jia-Ning Xiang, Shanghai (CN); Heng Xu, Shanghai (CN); Haibo Zhang, Shanghai (CN); Baowei Zhao, Shanghai (CN)

(73) Assignee: GLAXO GROUP LIMITED, Greenford, Middlesex (GB)

(21) Appl. No.: 13/379,431

(22) PCT Filed: Jun. 24, 2010
(86) PCT No.: PCT/CN10/00939

§ 371 Date: Dec. 20, 2011

(30) Foreign Application Priority Data
Jun. 26, 2009 (GB) 0911126.1
Oct. 21, 2009 (GB) 0918473.0

(51) Int. Cl. A61K 31/4439; C07D 413/04; C07D 271/107; C07D 285/12; C07D 285/08; C07D 277/30; C07D 417/04; A61K 31/4245; A61K 31/433; A61K 31/426; A61P 25/00; A61P 29/00; A61P 11/06; A61P 19/02; A61P 17/06; A61P 35/00; A61P 35/04; A61P 9/00; A61P 31/12; A61P 3/10; C07D 271/06

(52) U.S. Cl. 514/342; 548/131; 546/269.4; 548/145; 548/136; 548/128; 548/204; 546/270.4; 514/364; 514/340; 514/363; 514/361; 514/365

(57) ABSTRACT

5-membered heteroaryl derivatives of formula (I) or salts thereof, processes for their preparation, pharmaceutical compositions containing them and their uses in the treatment of various disorders mediated by S1P1 receptors are disclosed.

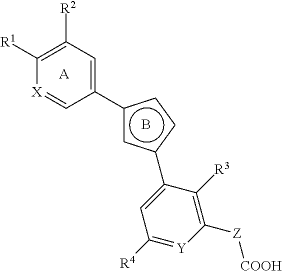

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandela et al 2002 Science 296:346, Fujino et al 2003 J, Pharmacology and Experimental Therapeutics 305:45658, Brinkman et al 2004 American J Transplantation 4:1019, Webb et al 2004 J Neuroimmunology 153:108, Morris et al 2005 Eur J Immuno) 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of SIPS knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO07/024,922, WO07/116,866, WO08/076,356, WO08/074,821, WO09/043,889, WO09/043,890, and U.S.09/007,6070.

The following patent application describes indole-oxadiazole derivatives as antipicornaviral agents: WO96/009822. The following patent applications describe indole-carboxylic acid derivatives as leukotriene receptor antagonists, pesticides and agrochemical fungicides respectively: WO06/090817, EP 0 439 785 and DE 39 39 238.

International patent applications WO08/074,821 and WO08/76356 describe oxadiazole-indole derivatives as S1P1 agonists.

The following patent applications describe oxadiazole and other 5-membered heteroaromatic ring derivatives as S1P1 agonists: WO05/082089, WO06/131336, WO08/029,306, WO08/029,370, and WO09/011,850.

The Chemical Abstracts Registry disloses a compound of 3-(3-(5-(3-cyano-4-isopropoxyphenyl)-1,2,4-oxadiazol-3-yl)-5-methylphenyl)propanoic acid without any associated activity or use (Chem Abs 1025928-99-7, 6 Jun. 2008).

A structurally novel class of compounds has now been found which provides agonists of the S1P1 receptor.

The present invention therefore provides as novel compounds of formula (I) or a salt thereof:

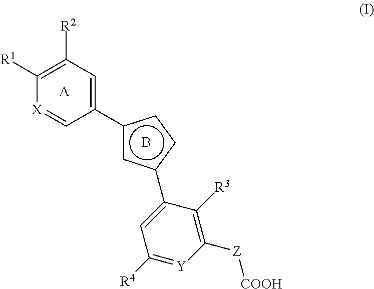

wherein

### X is CH or N;

### Y is CR⁵ or N;

Z is C₍₀₋₅₎alkyl optionally interrupted by O, S or N, and optionally substituted by C₍₀₋₃₎alkyl or OH;
B is a 5-membered heteroaryl ring selected from:

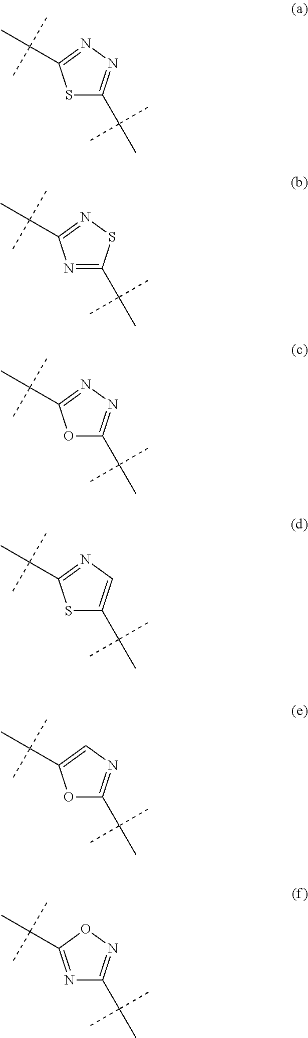

R¹ is C₍₁₋₄₎alkoxy;
R² is cyano or chloro;
R³ is hydrogen, halogen, trifluoromethyl, C₍₁₋₅₎alkyl, C₍₁₋₅₎alkoxy or CN;
R⁴ is hydrogen, halogen, trifluoromethyl, C₍₁₋₃₎alkyl or C₍₁₋₃₎alkoxy; and
R⁵ is hydrogen, halogen, trifluoromethyl, C₍₁₋₃₎alkyl or C₍₁₋₃₎alkoxy, with the proviso that the compound is not 3-(3-(5-(3-cyano-4-isopropoxyphenyl)-1,2,4-oxadiazol-3-yl)-5-methylphenyl)propanoic acid.

In one embodiment X is CH. In another embodiment X is N.

In one embodiment Y is CR⁵, wherein R⁵ is hydrogen or halogen. In another embodiment Y is CR⁵, wherein R⁵ is hydrogen or fluorine. In a further embodiment R⁵ is hydrogen. In a further embodiment Y is N.

In one embodiment Z is C₍₂₋₅₎alkyl optionally interrupted by O and optionally substituted by C₍₁₋₃₎alkyl. In another embodiment Z is C₍₂₋₅₎alkyl optionally substituted by methyl. In a further embodiment Z is OC₍₁₎alkyl.

In one embodiment Z is C₍₀₋₅₎alkyl. In another embodiment Z is C₍₂₎alkyl substituted by methyl. In a further embodiment Z is OC₍₁₋₃₎alkyl. In another embodiment Z is C₍₁₋₂₎NC₍₁₋₂₎alkyl optionally substituted by methyl on the N atom.

In one embodiment B is (a), (b), (c), (d) or (f).

In one embodiment B is (f).

In one embodiment R¹ is C₍₁₋₄₎alkoxy. In another embodiment R¹ is ispropoxy.

In one embodiment R² is cyano. In another embodiment R² is chloro.

In one embodiment R³ is hydrogen, methyl, ethyl, propyl, methoxy, chloro or trifluoromethyl.

In one embodiment R⁴ is hydrogen or fluoro.

In one embodiment R⁵ is hydrogen.

In one embodiment

### X is CH or N;

### Y is CR⁵ or N;

Z is C₍₂₋₅₎alkyl optionally interrupted by O and optionally substituted by C₍₁₋₃₎alkyl;

### B is (f);

R¹ is C₍₁₋₄₎alkoxy;
R² is cyano or chloro;
R³ is hydrogen, methyl, ethyl, methoxy, chloro or trifluoromethyl;
R⁴ is hydrogen; and
R⁵ is hydrogen.

In another embodiment

### X is CH or N;

### Y is CR⁵ or N;

Z is C₍₂₋₅₎alkyl optionally substituted by methyl or Z is OC₍₁₋₃₎alkyl;

### B is (f);

R¹ is ispropoxy;
R² is cyano or chloro;
R³ is hydrogen, methyl, ethyl, methoxy, chloro or trifluoromethyl;
R⁴ is hydrogen; and
R⁵ is hydrogen.

In one embodiment

### X is CH or N;

### Y is CR⁵ or N;

Z is C₍₀₋₅₎alkyl optionally interrupted by O or N and optionally substituted by C₍₁₋₃₎alkyl;

### B is (a), (b), (c), (d) or (f);

R¹ is C₍₁₋₄₎alkoxy;
R² is cyano or chloro;
R³ is hydrogen, methyl, ethyl, propyl, methoxy, chloro or trifluoromethyl;
R⁴ is hydrogen or fluoro; and
R⁵ is hydrogen.

In another embodiment

### X is CH or N;

### Y is CR⁵ or N;

Z is C₍₀₋₅₎alkyl, C₍₂₎alkyl substituted by methyl, OC₍₁₋₃₎alkyl or C₍₁₋₂₎NC₍₁₋₂₎alkyl optionally substituted by methyl on the N atom.

### B is (a), (b), (c), (d) or (f);

R¹ is ispropoxy;
R² is cyano or chloro;
R³ is hydrogen, methyl, ethyl, propyl, methoxy, chloro or trifluoromethyl;
R⁴ is hydrogen or fluoro; and
R⁵ is hydrogen.

The term “alkyl” as a group or part of a group e.g. alkoxy or hydroxyalkyl refers to a straight or branched alkyl group in all isomeric forms. The term “C₍₁₋₆₎ alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

As used herein, the term “halogen” refers to fluorine (F), chlorine (Cl), bromine (Br), or iodine (I) and the term “halo” refers to the halogen: fluoro (—F), chloro (—Cl), bromo (—Br) and iodo (—I).

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

Suitable compounds of formula (I) are:
3-[3-(5-{3-chloro-4-[(1-methylethyl)oxy]phenyl}-1,2,4-oxadiazol-3-yl)-2-methylphenyl]propanoic acid
4-[3-(5-{3-chloro-4-[(1-methylethyl)oxy]phenyl}-1,2,4-oxadiazol-3-yl)-2-methylphenyl]butanoic acid
(2R)-3-[3-(5-{5-chloro-6-[(1-methylethyl)oxy]-3-pyridinyl}-1,2,4-oxadiazol-3-yl)-2-methylphenyl]-2-methylpropanoic acid
4-[3-(5-{5-chloro-6-[(1-methylethyl)oxy]-3-pyridinyl}-1,2,4-oxadiazol-3-yl)-2-methylphenyl]butanoic acid
3-[3-(5-{3-cyano-4-[(1-methylethyl)oxy]phenyl}-1,2,4-oxadiazol-3-yl)-2-methylphenyl]propanoic acid
4-[3-(5-{3-cyano-4-[(1-methylethyl)oxy]phenyl}-1,2,4-oxadiazol-3-yl)-2-methylphenyl]butanoic acid
4-[4-(5-{3-chloro-4-[(1-methylethyl)oxy]phenyl}-1,2,4-oxadiazol-3-yl)-3-methyl-2-pyridinyl]butanoic acid
3-[2-chloro-3-(5-{5-chloro-6-[(1-methylethyl)oxy]-3-pyridinyl}-1,2,4-oxadiazol-3-yl)phenyl]propanoic acid
4-[2-chloro-3-(5-{5-chloro-6-[(1-methylethyl)oxy]-3-Pyridinyl}-1,2,4-oxadiazol-3-yl)phenyl]butanoic acid
3-[2-chloro-3-(5-{3-cyano-4-[(1-methylethyl)oxy]phenyl}-1,2,4-oxadiazol-3-yl)phenyl]propanoic acid
4-[2-chloro-3-(5-{3-cyano-4-[(1-methylethyl)oxy]phenyl}-1,2,4-oxadiazol-3-yl)phenyl]butanoic acid
6-[2-chloro-3-(5-{3-cyano-4-[(1-methylethyl)oxy]phenyl}-1,2,4-oxadiazol-3-yl)phenyl]hexanoic acid
3-[3-(5-{5-chloro-6-[(1-methylethyl)oxy]-3-pyridinyl}-1,2,4-oxadiazol-3-yl)-2-ethylphenyl]propanoic acid
4-[3-(5-{5-chloro-6-[(1-methylethyl)oxy]-3-pyridinyl}-1,2,4-oxadiazol-3-yl)-2-ethylphenyl]butanoic acid
4-[3-(5-{3-cyano-4-[(1-methylethyl)oxy]phenyl}-1,2,4-oxadiazol-3-yl)-2-ethylphenyl]butanoic acid
3-[3-(5-{3-chloro-4-[(1-methylethyl)oxy]phenyl}-1,2,4-oxadiazol-3-yl)-2-(methyloxy)phenyl]propanoic acid
4-[3-(5-{3-chloro-4-[(1-methylethyl)oxy]phenyl}-1,2,4-oxadiazol-3-yl)-2-(methyloxy)phenyl]butanoic acid
3-[3-(5-{3-cyano-4-[(1-methylethyl)oxy]phenyl}-1,2,4-oxadiazol-3-yl)-2-(methyloxy)phenyl]propanoic acid
4-[3-(5-{3-cyano-4-[(1-methylethyl)oxy]phenyl}-1,2,4-oxadiazol-3-yl)-2-(methyloxy)phenyl]butanoic acid
3-[4-(5-{3-cyano-4-[(1-methylethyl)oxy]phenyl}-1,2,4-oxadiazol-3-yl)-2-pyridinyl]propanoic acid
4-[4-(5-{3-cyano-4-[(1-methylethyl)oxy]phenyl}-1,2,4-oxadiazol-3-yl)-2-pyridinyl]butanoic acid
3-[5-(5-{3-cyano-4-[(1-methylethyl)oxy]phenyl}-1,2,4-oxadiazol-3-yl)-2-fluorophenyl]propanoic acid
4-[5-(5-{3-cyano-4-[(1-methylethyl)oxy]phenyl}-1,2,4-oxadiazol-3-yl)-2-fluorophenyl]butanoic acid
3-[3-(5-{3-cyano-4-[(1-methylethyl)oxy]phenyl}-1,2,4-oxadiazol-3-yl)-2-(trifluoromethyl)phenyl]propanoic acid
4-[3-(5-{3-cyano-4-[(1-methylethyl)oxy]phenyl}-1,2,4-oxadiazol-3-yl)-2-(trifluoromethyl)phenyl]butanoic acid
3-(5-{3-cyano-4-[(1-methylethyl)oxy]phenyl}-1,2,4-oxadiazol-3-yl)-2-methylbenzoic acid
{[3-(5-{5-chloro-6-[(1-methylethyl)oxy]-3-pyridinyl}-1,2,4-oxadiazol-3-yl)-2-methylphenyl]oxy}acetic acid
4-[3-(5-{3-chloro-4-[(1-methylethyl)oxy]phenyl}-1,2,4-oxadiazol-3-yl)-2-ethylphenyl]butanoic acid
3-[3-(5-{3-chloro-4-[(1-methylethyl)oxy]phenyl}-1,2,4-oxadiazol-3-yl)-2-ethylphenyl]propanoic acid
3-[3-(5-{3-cyano-4-[(1-methylethyl)oxy]phenyl}-1,2,4-oxadiazol-3-yl)-5-fluoro-2-(methyloxy)phenyl]propanoic acid
4-[3-(5-{3-cyano-4-[(1-methylethyl)oxy]phenyl}-1,2,4-oxadiazol-3-yl)-5-fluoro-2-(methyloxy)phenyl]butanoic acid
4-[3-(5-{5-chloro-6-[(1-methylethyl)oxy]-3-pyridinyl}-1,2,4-oxadiazol-3-yl)-5-fluoro-2-(methyloxy)phenyl]butanoic acid
3-[3-(5-{5-chloro-6-[(1-methylethyl)oxy]-3-pyridinyl}-1,2,4-oxadiazol-3-yl)-5-fluoro-2-(methyloxy)phenyl]propanoic acid
3-[3-(5-{3-chloro-4-[(1-methylethyl)oxy]phenyl}-1,2,4-oxadiazol-3-yl)-5-fluoro-2-(methyloxy)phenyl]propanoic acid
4-[3-(5-{3-chloro-4-[(1-methylethyl)oxy]phenyl}-1,2,4-oxadiazol-3-yl)-5-fluoro-2-(methyloxy)phenyl]butanoic acid
4-[3-(5-{3-chloro-4-[(1-methylethyl)oxy]phenyl}-1,2,4-oxadiazol-3-yl)-2-propylphenyl]butanoic acid
4-[3-(5-{3-cyano-4-[(1-methylethyl)oxy]phenyl}-1,2,4-oxadiazol-3-yl)-2-propylphenyl]butanoic acid
4-{[3-(5-{3-cyano-4-[(1-methylethyl)oxy]phenyl}-1,2,4-oxadiazol-3-yl)-2-ethylphenyl]oxy}butanoic acid
4-{[3-(5-{3-chloro-4-[(1-methylethyl)oxy]phenyl}-1,2,4-oxadiazol-3-yl)-2-ethylphenyl]oxy}butanoic acid
{[3-(5-{3-cyano-4-[(1-methylethyl)oxy]phenyl}-1,2,4-oxadiazol-3-yl)-2-ethylphenyl]oxy}acetic acid
4-[3-(5-{3-cyano-4-[(1-methylethyl)oxy]phenyl}-1,3,4-oxadiazol-2-yl)-2-ethylphenyl]butanoic acid
4-[3-(5-{3-cyano-4-[(1-methylethyl)oxy]phenyl}-1,3,4-thiadiazol-2-yl)-2-ethylphenyl]butanoic acid
N-{[3-(5-{3-chloro-4-[(1-methylethyl)oxy]phenyl}-1,3,4-thiadiazol-2-yl)-2-ethylphenyl]methyl}-N-methylglycine
4-[3-(3-{3-cyano-4-[(1-methylethyl)oxy]phenyl}-1,2,4-thiadiazol-5-yl)-2-ethylphenyl]butanoic acid
4-[3-(5-{3-chloro-4-[(1-methylethyl)oxy]phenyl}-1,2,4-thiadiazol-3-yl)-2-ethylphenyl]butanoic acid
4-[3-(5-{3-cyano-4-[(1-methylethyl)oxy]phenyl}-1,2,4-thiadiazol-3-yl)-2-ethylphenyl]butanoic acid
N-{[3-(5-{3-cyano-4-[(1-methylethyl)oxy]phenyl}-1,2,4-thiadiazol-3-yl)-2-ethylphenyl]methyl}-N-methylglycine
N-{[3-(5-{3-cyano-4-[(1-methylethyl)oxy]phenyl}-1,2,4-thiadiazol-3-yl)-2-ethylphenyl]methyl}-b-alanine
N-{2-[3-(5-{3-chloro-4-[(1-methylethyl)oxy]phenyl}-1,2,4-thiadiazol-3-yl)-2-ethylphenyl]ethyl}-N-methylglycine
4-[3-(2-{3-cyano-4-[(1-methylethyl)oxy]phenyl}-1,3-thiazol-5-yl)-2-ethylphenyl]butanoic acid
4-[3-(2-{3-cyano-4-[(1-methylethyl)oxy]phenyl}-1,3-thiazol-5-yl)-5-fluoro-2-(methyloxy)phenyl]butanoic acid
4-[3-(2-{3-chloro-4-[(1-methylethyl)oxy]phenyl}-1,3-thiazol-5-yl)-2-ethylphenyl]butanoic acid
N-{2-[3-(2-{3-cyano-4-[(1-methylethyl)oxy]phenyl}-1,3-thiazol-5-yl)-2-ethylphenyl]ethyl}-N-methylglycine
N-{[3-(2-{3-chloro-4-[(1-methylethyl)oxy]phenyl}-1,3-thiazol-5-yl)-2-ethylphenyl]methyl}-N-methylglycine
N-{[3-(2-{3-cyano-4-[(1-methylethyl)oxy]phenyl}-1,3-thiazol-5-yl)-2-ethylphenyl]methyl}-N-methylglycine
N-{2-[3-(2-{3-chloro-4-[(1-methylethyl)oxy]phenyl}-1,3-thiazol-5-yl)-2-ethylphenyl]ethyl}-N-methylglycine
N-{[3-(2-{3-cyano-4-[(1-methylethyl)oxy]phenyl}-1,3-thiazol-5-yl)-2-ethylphenyl]methyl}-N-methyl-β-alanine
4-[4-(2-{3-cyano-4-[(1-methylethyl)oxy]phenyl}-1,3-thiazol-5-yl)-3-ethyl-2-pyridinyl]butanoic acid
N-{2-[3-(2-{3-cyano-4-[(1-methylethyl)oxy]phenyl}-1,3-thiazol-5-yl)-2-ethylphenyl]ethyl}-N-methyl-b-alanine
3-[3-(5-{3-chloro-4-[(1-methylethyl)oxy]phenyl}-1,2,4-oxadiazol-3-yl)phenyl]propanoic acid
   or salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

In a further aspect, this invention provided processes for the preparation of a compound of formula (I). In one aspect, certain compounds of formula (I) were prepared by the process in Schemes I to XIV.

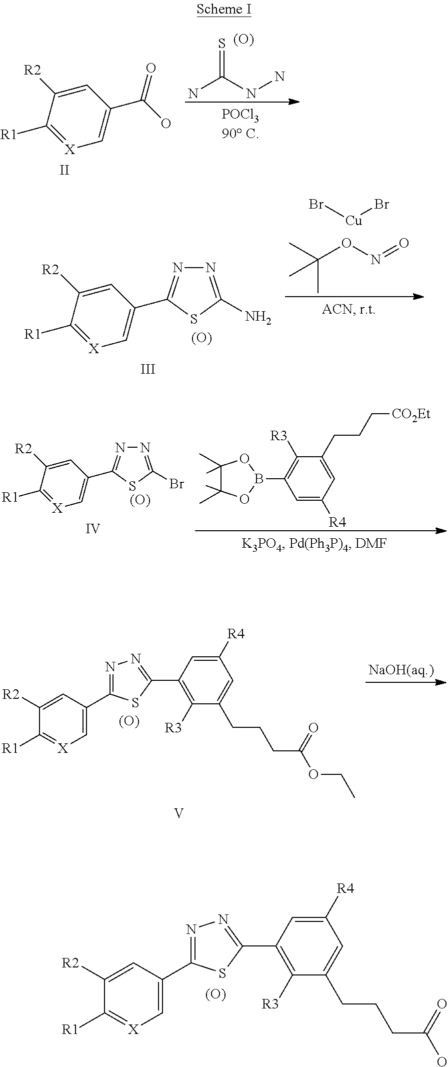

The first step of the process (II to III) is carried out in POCl₃ at 90° C. In the second step of the process (III to IV) suitable reagents include CuBr₂ and 1,1-dimethylethyl nitrite were used at low temperature (0° C.). The Suzuki coupling reaction (IV to V) may be performed employing suitable reagents including Pd(PPh₃)₄ and K₃PO₄ in a suitable solvent such as DMF or DME under microwave condition. The last step of the process (V to I) is carried out by treatment with basic (such as sodium hydroxide in a suitable solvent such as isopropanol) conditions and may be carried out at room temperature.

In another aspect, compounds of formula (I) can be prepared by the process in Scheme II to XIV.

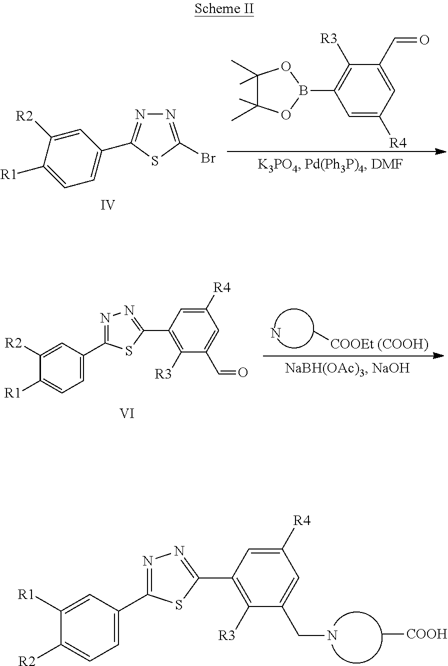

The Suzuki coupling reation in scheme II (IV to VI) is similar as that described in scheme I. In the next step, intermediate (VI) can be converted to (I) by reductive amination reaction with amino acids in the presence of a suitable reducing reagent such as NaBH(OAc)₃ in a suitable solvent (methanol or CH₂Cl₂) at room temperature. If the amino ester (instead of the amino acid) was used for the reductive amination process, the ester should be hydrolyzed to the corresponding acid by treatment with basic (such as sodium hydroxide in a suitable solvent such as isopropanol) conditions to get compound I.

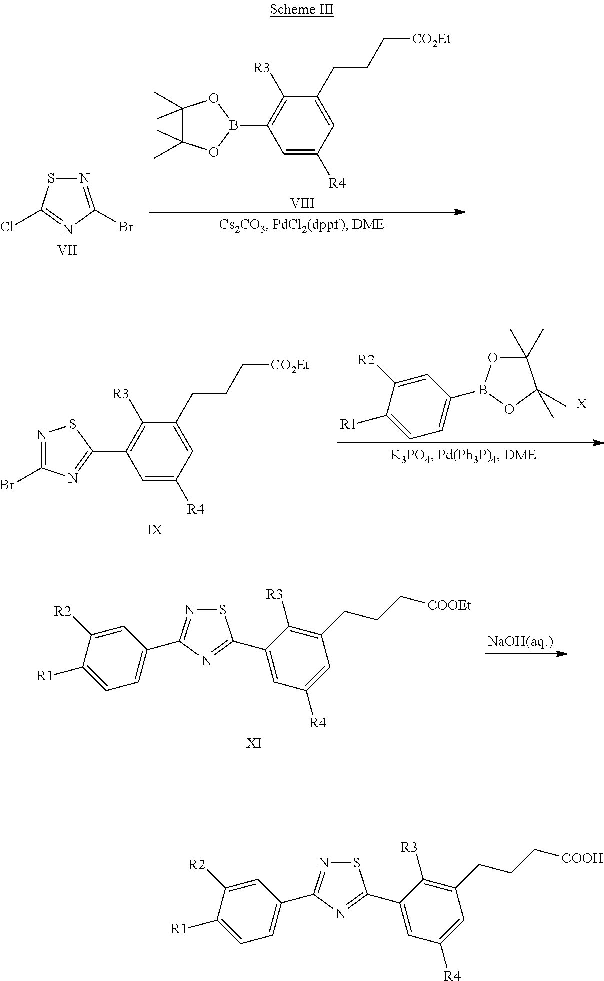

In scheme III, the first two steps (VII to XI) are Suzuki coupling reactions, and suitable palladium catalysts (such as PdCl₂(dppf) and Pd(PPh₃)₄), bases (such as Cs₂CO₃ and K₃PO₄), and solvents (such as DMF and DME) need to be selected to provide good yield. Representative compounds of formula (VIII) can be prepared as described in the experimental section. Hydrolysis of the ethyl ester in intermediate XI provide compound I.

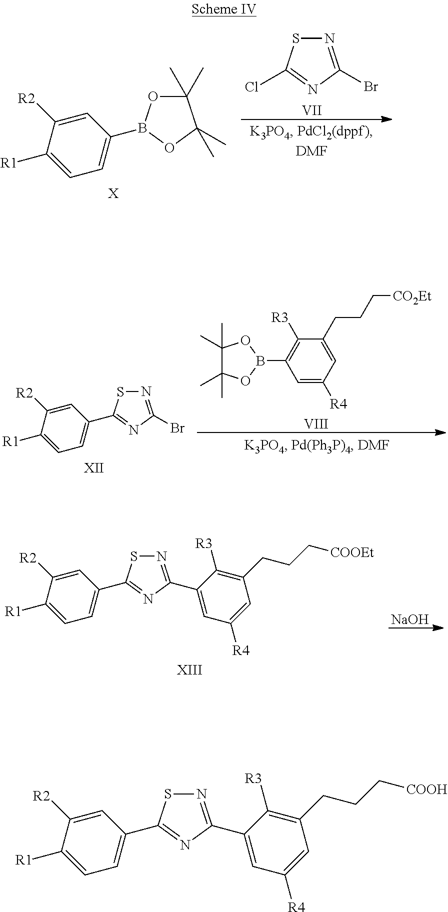

Steps in scheme IV (X to XIII to I) are similar with process (VII to XI to I) in scheme III.

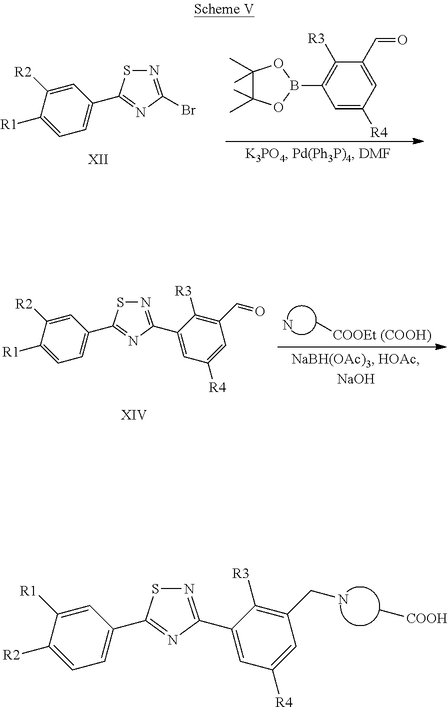

The two steps (XII to XIV to I) in scheme V are similar with process (IV to VI to I) in scheme II.

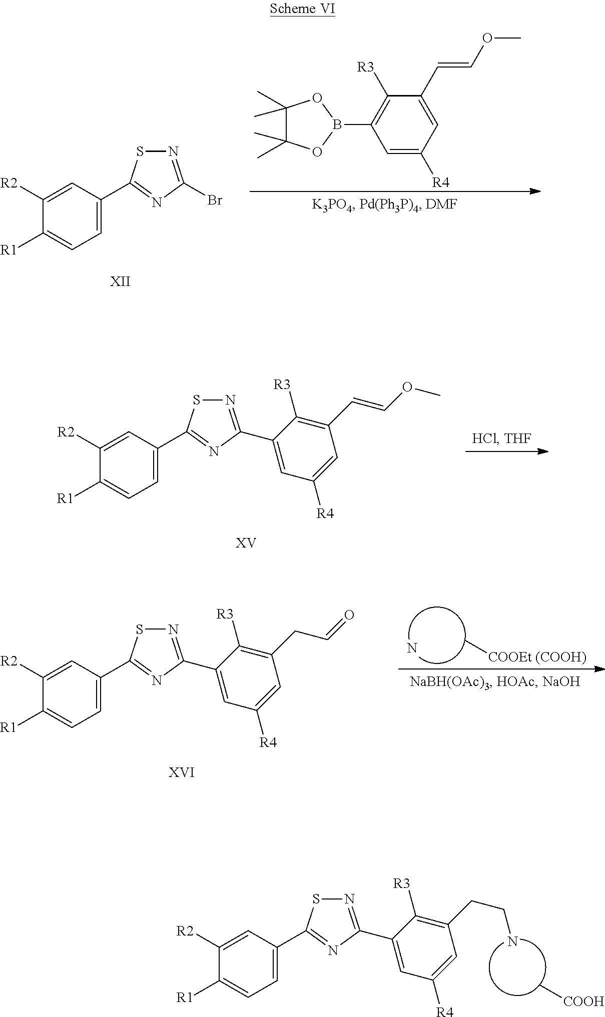

In the first step of Suzuki coupling process (XII to XV) suitale reagents include Pd(PPh₃)₄ and K₃PO₄ in a solvent such as DMF or DME under microwave condition. In the second step of the process (XV to XVI) suitable reagent HCl in suitable solvent dioxane or TMSCI in acetonitrile at room temperature. The last step (XVI to I) is similar with process (VI to I) in scheme II.

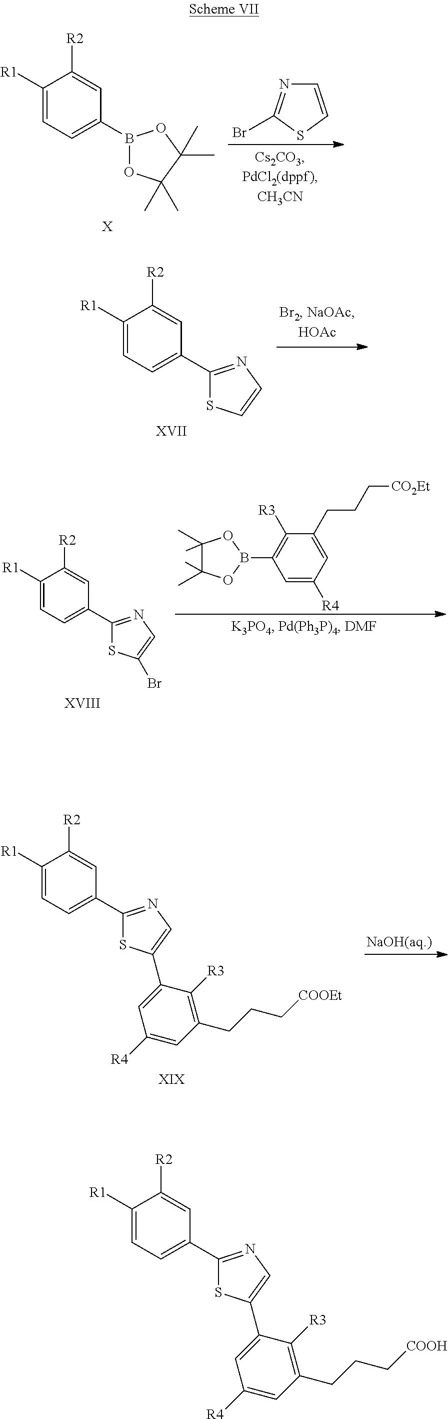

In the first step of Suzuki coupling process (X to XVII) suitale reagents include PdCl₂(dppf) and Cs₂CO₃ in a solvent such as CH₃CN or DME under microwave condition. The second step of the process (XVII to XVIII) is carried out by treatment with Br₂ in a suitable solvent such as HOAc at room temperature. The last two steps (XIX to I) are similar with process (IV to I) in scheme I.

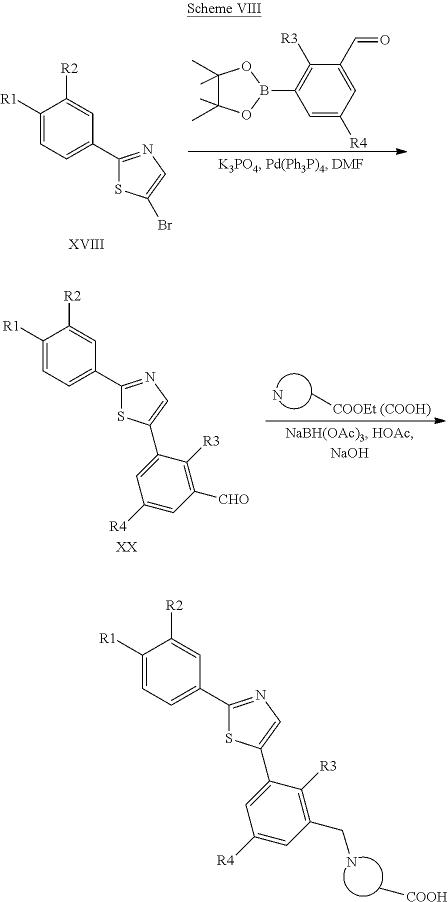

The two steps (XVIII to I) are similar with process (IV to I) in scheme II.

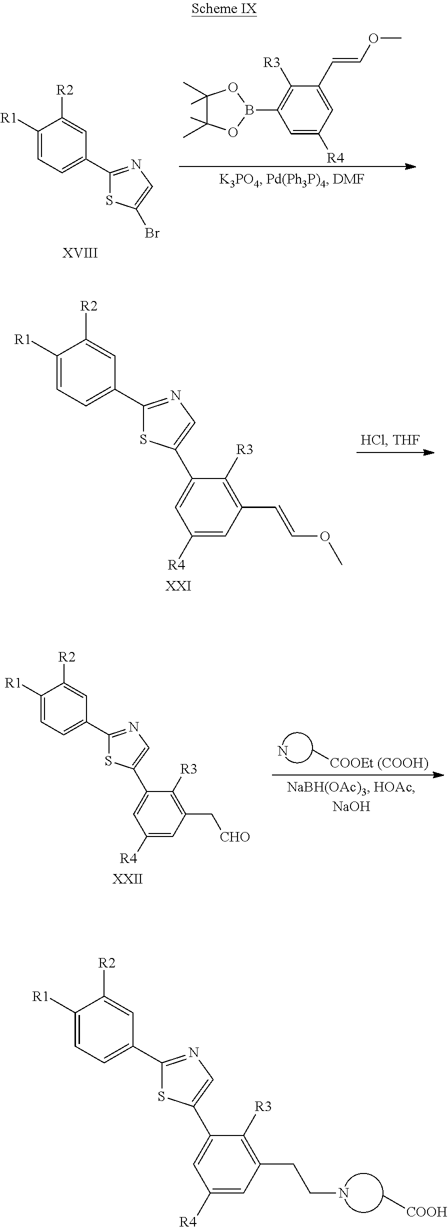

The two steps (XVIII to I) are similar with process (XII to I) in scheme VI.

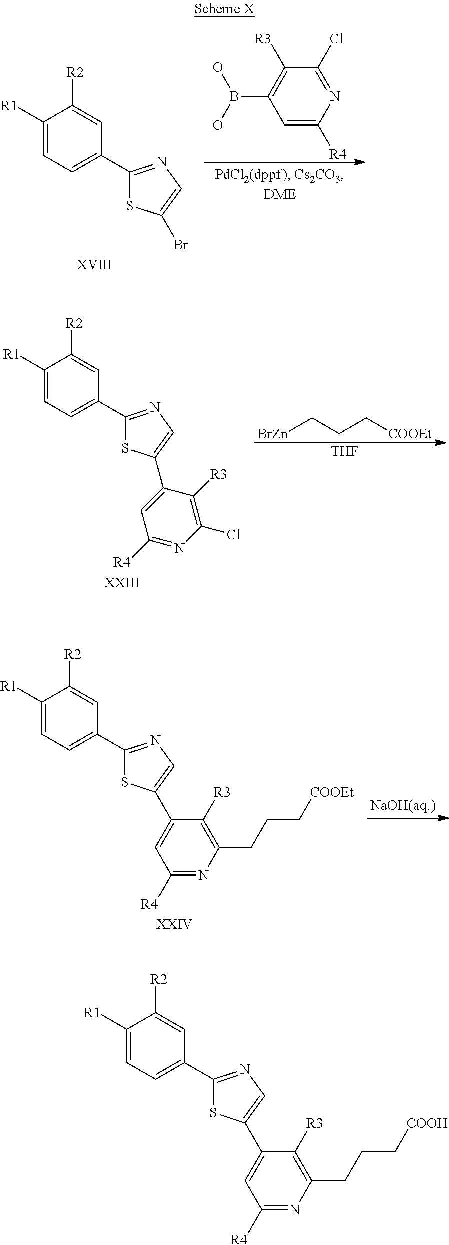

In the first step of Suzuki coupling process (XVIII to XXIII) suitale reagents include PdCl₂(dppf) and Cs₂CO₃ in a solvent such as DME under microwave condition. Formula (XXIII) can be converted to (XXIV) by treatment with suitable reagent BrZn(CH₂)₃COOEt in a suitable solvent such as THF at elevated temperature. The last step (XXIV to I) is similar with process (V to I) in scheme I.

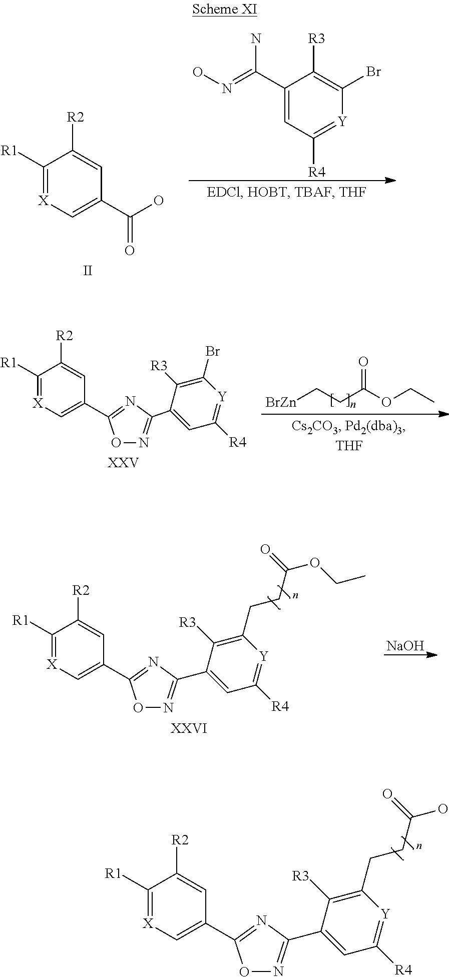

In the first step of the process (II to XXV) suitable reagents include EDCI, HOBT and TBAF. The second step of Negishi coupling process (XXV to XXVI) suitale reagents include Pd₂(dba)₃ and Cs₂CO₃ in a solvent such as THF. The last step of the process (XXVI to I) is carried out by treatment with basic (such as sodium hydroxide in a suitable solvent such as i-propanol or THF) conditions and may be carried out at 60-90° C.

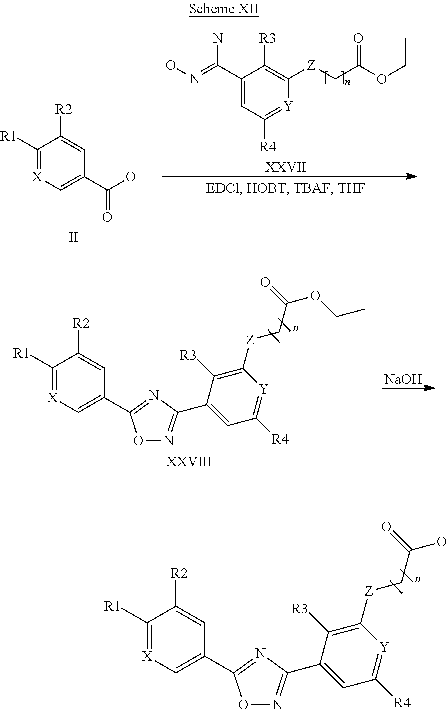

The first step of process (II to XXVIII) is similar with process (II to XXV) in scheme XI. Compounds of formula (XXVII) can be prepared as described in the experimental section. The last step of the process (XXVIII to I) is carried out by treatment with basic (such as sodium hydroxide in a suitable solvent such as isopropanol) conditions and may be carried out at room temperature.

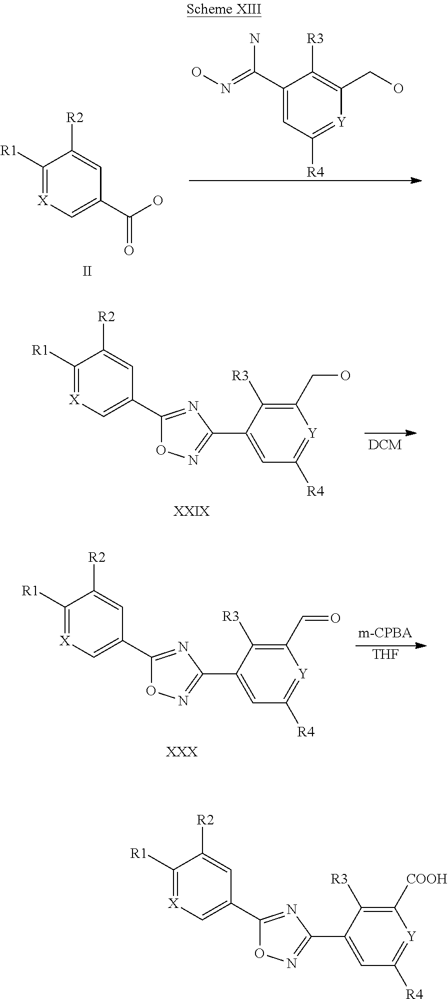

The first step of process (II to XXIX) is similar with process (II to XXV) in scheme XI. In the last two steps, formula (XXX) can be converted to (I) by oxidation in suitable conditions.

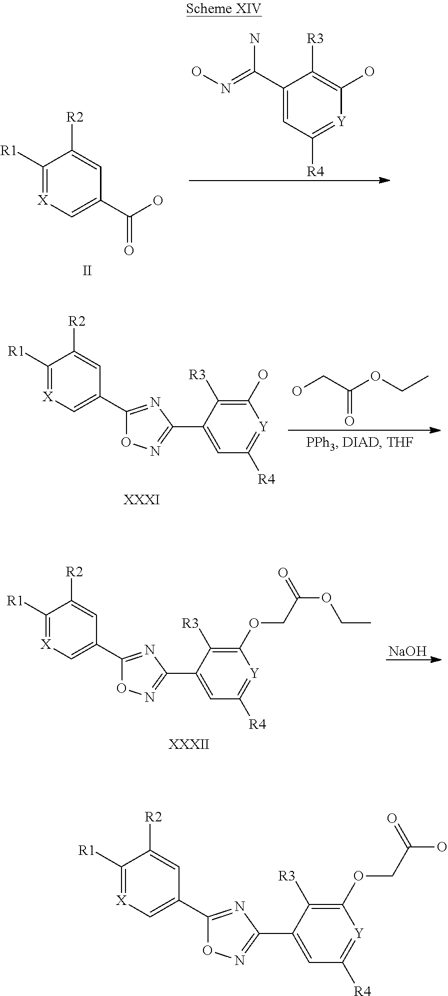

The first step of process (II to XXXI) is similar with process (II to XXV) in scheme XI. The second step of process (XXXI to XXXII) is carried out in suitable solvent such as THF with reagents include ethyl hydroxyacetate, PPh₃ and DIAD. The last step of the process (XXXII to I) is carried out by treatment with basic (such as sodium hydroxide in a suitable solvent such as i-propanol or THF) conditions and may be carried out at 50° C.

Pharmaceutically acceptable salts may be prepared conventionally by reaction with the appropriate acid or acid derivative.

The potencies and efficacies of the compounds of this invention for the S1P1 receptor can be determined by GTPγS assay or S1P1 Tango assay performed on the human cloned receptor as described herein. Compounds of formula (I) have demonstrated agonist activity at the S1P1 receptor, using functional assays described herein.

Compounds of formula (I) including 3-(3-(5-(3-cyano-4-isopropoxyphenyl)-1,2,4-oxadiazol-3-yl)-5-methylphenyl)propanoic acid and their pharmaceutically acceptable salts are therefore of use in the treatment of conditions or disorders which are mediated via the S1P1 receptor.

In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides a compound of formula (I) and a pharmaceutically acceptable salt thereof, for use as therapeutic substances, in particular in the treatment of the conditions or disorders mediated via the S1P1 receptor. In particular the invention provides a compound of formula (I) or a pharmaceutically acceptable salt thereof for use as therapeutic substances in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes. The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof.

Compounds of formula (I) and their pharmaceutically acceptable salts are of use as therapeutic substances in the treatment of multiple sclerosis.

In another aspect, the invention provides for the use of compounds of formula (I) or a pharmaceutically acceptable salt thereof in the manufacture of a medicament for use in the treatment of the conditions or disorders mediated via the S1P1 receptor

The invention provides a method of treatment of multiple sclerosis, which comprises administering to the sufferer a therapeutically safe and effective amount of compound of formula (I) or a pharmaceutically acceptable salt thereof.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) can generally be prepared by carrying out the procedures disclosed in the Schemes and/or in the Examples below, by substituting a readily available isotopically labelled reagent for a non-isotopically labeled reagent.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
min—minute
ul—microlitres
MeCN—acetonitrile
MeOH—methanol
EtOH—ethanol
Et₂O—diethyl ether
EtOAc—ethyl acetate
DABCO—1,4-diazabiclo[2,2,2]octane
DCM—dichloromethane
DIAD—diisopropyl azodicarboxylate
DME—1,2-bis(methyloxy)ethane

### DMF—N,N-dimethylformamide

DMSO—dimethylsuiphoxide
EDAC—N-(3-Dimethylaminopropyl)-N′-ethylcarbodiimide hydrochloride
EDC—N-(3-Dimethylaminopropyl)-N′-ethylcarbodiimide hydrochloride
EDCI—N-(3-Dimethylaminopropyl)-N′-ethylcarbodiimide hydrochloride

### HOBT/HOBt—Hydroxybenzotriazole

IPA—isopropylalcohol

### NCS—N-chlorosuccinimide

PyBOP—Benzotriazol-1-yl-oxytripyrrolidinophosphonium hexafluorophosphate
THF—tetrahydrofuran
dba—dibenzylidene acetone
RT—room temperature
° C.—degrees Celsius

### M—Molar

H—proton
s—singlet
d—doublet
t—triplet
q—quartet
MHz—megahertz
MeOD—deuterated methanol

### LCMS—Liquid Chromatography Mass Spectrometry

### LC/MS—Liquid Chromatography Mass Spectrometry

MS—mass spectrometry

### ES—Electrospray

MH⁺—mass ion+H⁺
MDAP—mass directed automated preparative liquid chromatography.
sat.—saturated

### Chromatography

Unless stated otherwise, all chromatography was carried out using silica columns.

### General Chemistry Section

The intermediates for the preparation of the examples may not necessarily have been prepared from the specific batch of precursor described.

### DESCRIPTION FOR D1

### 3-bromo-N-hydroxy-2-methylbenzenecarboximidamide (D1)

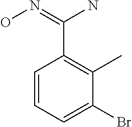

A mixture of 3-bromo-2-methylbenzonitrile (3.0 g), sodium bicarbonate (2.57 g) and hydroxylamine hydrochloride (2.13 g) in ethanol (100 ml) was stirred at 65° C. overnight. The inorganic precipitate was filtered off. The solid was washed thoroughly with ethanol. The filtrate was concentrated. The obtained solid was dried in vacuo to afford 3-bromo-N-hydroxy-2-methylbenzenecarboximidamide (D1) (3.27 g). MS (ES): C₈H₉BrN₂O requires 229; found 230.1 (M+H⁺).

### DESCRIPTION FOR D2

### 3-(3-bromo-2-methylphenyl)-5-{3-chloro-4-[(1-methylethyl)oxy]phenyl}-1,2,4-oxadiazole (D2)

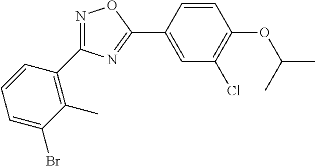

3-Chloro-4-[(1-methylethyl)oxy]benzoic acid (5.0 g), EDCI (11.16 g), HOBT (7.87 g) were dissolved in THF (100 ml). After the mixture was stirred for 10 mins at RT, a solution of 3-bromo-N-hydroxy-2-methylbenzenecarboximidamide (D1) (4.27 g) in THF (100 ml) was added slowly. The obtained mixture was stirred for further 2 hours at room temperature. LCMS show only one intermediate was found. After addition of TBAF (24.36 g), the reaction mixture was refluxed for 3 days. The organic phase was washed three times with water (50 mL), dried over sodium sulphate and evaporated in vacuo to give the crude product as an orange solid. The crude product was added to a silica gel column and was eluted with hexane:DCM=9:1LCMS confirmed as the desired compound 3-(3-bromo-2-methylphenyl)-5-{3-chloro-4-[(1-methylethyl)oxy]phenyl}-1,2,4-oxadiazole (D2) (5.55 g). MS (ES): C₁₈H₁₆BrClN₂O₂ requires 407; found 408.2 (M+H⁺).

### DESCRIPTION FOR D3

### ethyl 3-[3-(5-{3-chloro-4-[(1-methylethyl)oxy]phenyl}-1,2,4-oxadiazol-3-yl)-2-methylphenyl]propanoate (D3)

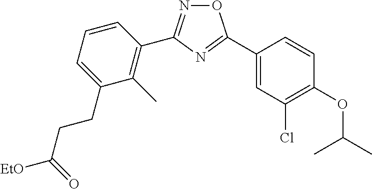

To a suspension of 3-(3-bromo-2-methylphenyl)-5-{3-chloro-4-[(1-methylethyl)oxy]phenyl}-1,2,4-oxadiazole (D2) (140 mg), Pd(PPh₃)₄ (60 mg) in THF (15 ml) stirred under nitrogen at room temperature was added a solution of bromo[3-(ethyloxy)-3-oxopropyl]zinc (0.5 M in THF, 5 ml) in one charge of 1 minute. The reaction mixture was stirred at 40° C. for 2 hours. The mixture was cooled to room temperature. The solvent was evaporated off and EtOAc (25 ml) was added. The organic phase was washed with 2M sodium hydroxide solution 25 ml (three times), dried over sodium sulphate and evaporated to afford ethyl 3-[3-(5-{3-chloro-4-[(1-methylethyl)oxy]phenyl}-1,2,4-oxadiazol-3-yl)-2-methylphenyl]propanoate (D3) (126 mg), applied to next step directly. MS (ES): C₂₃H₂₅ClN₂O₄ requires 429; found 430.1 (M+H⁺).

### DESCRIPTION FOR D4

### ethyl 4-[3-(5-{3-chloro-4-[(1-methylethyl)oxy]phenyl}-1,2,4-oxadiazol-3-yl)-2-methylphenyl]butanoate (D4)

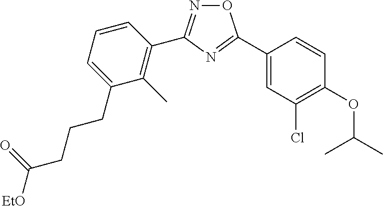

To a suspension of 3-(3-bromo-2-methylphenyl)-5-{3-chloro-4-[(1-methylethyl)oxy]phenyl}-1,2,4-oxadiazole (D2) (140 mg), Pd(PPh₃)₄ (64 mg) in THF (15 ml) stirred under nitrogen at room temperature was added a solution of bromo[4-(ethyloxy)-4-oxobutyl]zinc (0.5 M in THF, 2 ml) in one portion. The reaction mixture was stirred at 40° C. for 2 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-chloro-4-[(1-methylethyl)oxy]phenyl}-1,2,4-oxadiazol-3-yl)-2-methylphenyl]butanoate (D4) (141 mg), applied to next step directly. MS (ES): C₂₄H₂₇ClN₂O₄ requires 443; found 444.1 (M+H⁺).

### DESCRIPTION FOR D5

### 5-[3-(3-bromo-2-methylphenyl)-1,2,4-oxadiazol-5-yl]-3-chloro-2-[(1-methylethyl)oxy]pyridine (D5)

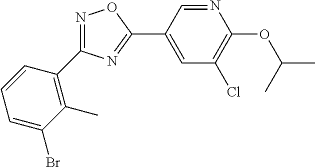

5-chloro-6-[(1-methylethyl)oxy]-3-pyridinecarboxylic acid (6.65 g), EDCI (14.78 g), HOBT (10.42 g) were dissolved in THF (100 ml). After the mixture was stirred for 10 mins at RT, a solution of 3-bromo-N-hydroxy-2-methylbenzenecarboximidamide (D1) (4.95 g) in THF (50 ml) was added slowly. The obtained mixture was stirred for further 2 hours at room temperature. LCMS show only one intermediate was found. After addition of TBAF (32.3 g), the reaction mixture was heated to 80° C. for 2 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (Hexane:DCM=9:1) to afford a off white solid. LCMS confirmed as the desired compound 5-[3-(3-bromo-2-methylphenyl)-1,2,4-oxadiazol-5-yl]-3-chloro-2-[(1-methylethyl)oxy]pyridine (D5) (6.86 g). δH (CDCl₃, 400 MHz): 1.46 (6H, d), 2.49 (3H, s), 4.71 (1H, m), 7.38 (1H, d), 8.00 (1H, dd), 8.35 (1H, d), 8.40 (1H, d), 8.88 (1H, d). MS (ES): C₁₇H₁₅BrClN₃O₂ requires 407; found 408.2 (M+H⁺).

### DESCRIPTION FOR D6

### methyl (2R)-3-[3-(5-{5-chloro-6-[(1-methylethyl)oxy]-3-pyridinyl}-1,2,4-oxadiazol-3-yl)-2-methylphenyl]-2-methylpropanoate (D6)

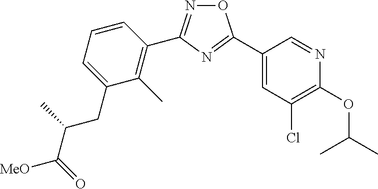

To a suspension of 5-[3-(3-bromo-2-methylphenyl)-1,2,4-oxadiazol-5-yl]-3-chloro-2-[(1-methylethyl)oxy]pyridine (138 mg), Pd(PPh₃)₄ (59 mg) in THF (15 ml) stirred under nitrogen at room temperature was added a solution of bromo[2-methyl-3-(methyloxy)-3-oxopropyl]zinc (0.5 M in THF, 3.4 ml) in one portion. The reaction mixture was stirred at 60° C. for 3 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford methyl (2R)-3-[3-(5-{5-chloro-6-[(1-methylethyl)oxy]-3-pyridinyl}-1,2,4-oxadiazol-3-yl)-2-methylphenyl]-2-methylpropanoate (146 mg), applied to next step directly. MS (ES): C₂₃H₂₅ClN₂O₄ requires 429; found 430.1 (M+H⁺).

### DESCRIPTION FOR D7

### ethyl 4-[3-(5-{5-chloro-6-[(1-methylethyl)oxy]-3-pyridinyl}-1,2,4-oxadiazol-3-yl)-2-methylphenyl]butanoate (D7)

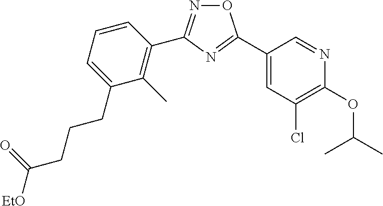

To a suspension of 5-[3-(3-bromo-2-methylphenyl)-1,2,4-oxadiazol-5-yl]-3-chloro-2-[(1-methylethyl)oxy]pyridine (300 mg), Pd(PPh₃)₄ (127 mg) in THF (15 ml) stirred under nitrogen at room temperature was added a solution of bromo[4-(ethyloxy)-4-oxobutyl]zinc (0.5 M in THF, 12 ml) in one portion. The reaction mixture was stirred at 50° C. for 0.5 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{5-chloro-6-[(1-methylethyl)oxy]-3-pyridinyl}-1,2,4-oxadiazol-3-yl)-2-methylphenyl]butanoate (231 mg), applied to next step directly. MS (ES): C₂₃H₂₆ClN₃O₄ requires 444; found 445.1 (M+H⁺).

### DESCRIPTION FOR D8

### 5-[3-(3-bromo-2-methylphenyl)-1,2,4-oxadiazol-5-yl]-2-[(1-methylethyl)oxy]benzonitrile (D8)

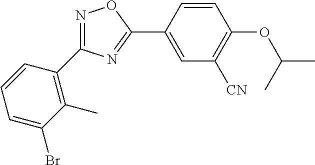

3-cyano-4-[(1-methylethyl)oxy]benzoic acid (0.8 g), EDCI (1.49 g), HOBT (1.19 g) were dissolved in THF (10 mL). After the mixture was stirred for 10 mins at RT, a solution of 3-bromo-N-hydroxy-2-methylbenzenecarboximidamide (0.84 g) in THF (10 ml) was added slowly. The obtained mixture was stirred for further 2 hours at room temperature. LCMS show only one intermediate was found. After addition of TBAF (3.73 g), the reaction mixture was heated to 80° C. for 2 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=10:1) to afford a off white solid. LCMS confirmed as the desired compound 5-[3-(3-bromo-2-methylphenyl)-1,2,4-oxadiazol-5-yl]-2-[(1-methylethyl)oxy]benzonitrile (0.96 g). MS (ES): C₁₉H₁₆BrN₃O₂ requires 397; found 398.2 (M+H⁺).

### DESCRIPTION FOR D9

### ethyl 3-[3-(5-{3-cyano-4-[(1-methylethyl)oxy]phenyl}-1,2,4-oxadiazol-3-yl)-2-methylphenyl]propanoate (D9)

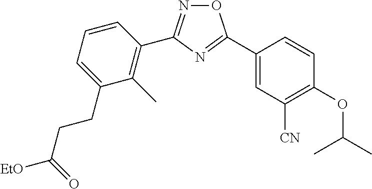

To a suspension of 5-[3-(3-bromo-2-methylphenyl)-1,2,4-oxadiazol-5-yl]-2-[(1-methylethyl)oxy]benzonitrile (150 mg), Pd₂(dba)₃ (52 mg), tris(1,1-dimethylethyl)phosphane (33 mg), Cs₂CO₃ (37 mg) in THF (5 ml) stirred under nitrogen at room temperature was added a solution of bromo[3-(ethyloxy)-3-oxopropyl]zinc (0.5 M in THF, 2 ml) in one portion. The reaction mixture was stirred at room temperature for 1.5 hours. Evaporate off the solvent, EtOAc (25 ml) was added. The organic phase was washed with 2M sodium hydroxide solution 25 ml (three times), dried over sodium sulphate and evaporated to afford ethyl 3-[3-(5-{3-cyano-4-[(1-methylethyl)oxy]phenyl}-1,2,4-oxadiazol-3-yl)-2-methylphenyl]propanoate (146 mg), applied to next step directly. MS (ES): C₂₄H₂₅N₃O₄ requires 419; found 420.1 (M+H⁺).

### DESCRIPTION FOR D10

### ethyl 4-[3-(5-{3-cyano-4-[(1-methylethyl)oxy]phenyl}-1,2,4-oxadiazol-3-yl)-2-methylphenyl]butanoate (D10)

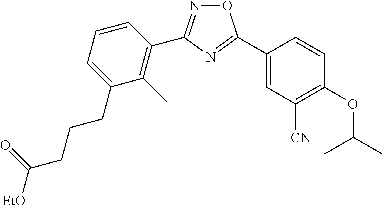

To a suspension of 5-[3-(3-bromo-2-methylphenyl)-1,2,4-oxadiazol-5-yl]-2-[(1-methylethyl)oxy]benzonitrile (150 mg), Pd₂(dba)₃ (52 mg), tris(1,1-dimethylethyl)phosphane (33 mg), Cs₂CO₃ (37 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 1.5 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-cyano-4-[(1-methylethyl)oxy]phenyl}-1,2,4-oxadiazol-3-yl)-2-methylphenyl]butanoate (153 mg), applied to next step directly. MS (ES): C₂₅H₂₇N₃O₄ requires 433; found 434.1 (M+H⁺).

### DESCRIPTION FOR D11

### 2-bromo-N-hydroxy-3-methyl-4-pyridinecarboximidamide (D11)

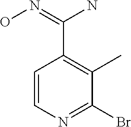

A mixture of 2-bromo-3-methyl-4-pyridinecarbonitrile (3.0 g), sodium bicarbonate (2.57 g) and hydroxylamine hydrochloride (2.13 g) in ethanol (100 ml) was heated at reflux for overnight. The inorganic precipitate was filtered off. The solid was washed thoroughly with ethanol. The filtrate was concentrated. The obtained solid was dried in vacuo to afford 2-bromo-N-hydroxy-3-methyl-4-pyridinecarboximidamide (3.27 g). MS (ES): C₇H₈BrN₂O requires 230; found 231.1 (M+H⁺).

### DESCRIPTION FOR D12

### 2-bromo-4-(5-{3-chloro-4-[(1-methylethyl)oxy]phenyl}-1,2,4-oxadiazol-3-yl)-3-methylpyridine (D12)

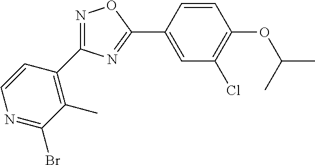

3-Chloro-4-[(1-methylethyl)oxy]benzoic acid (0.5 g), EDCI (1.16 g), HOBT (0.8 g) were dissolved in THF (10 ml). After the mixture was stirred for 10 mins at RT, a solution of 2-bromo-N-hydroxy-3-methyl-4-pyridinecarboximidamide (0.32 g) in THF (50 ml) was added slowly. The obtained mixture was stirred for further 2 hours at room temperature. LCMS show only one intermediate was found. After addition of TBAF (2.4 g), the reaction mixture was heated to 80° C. for 1 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (Hexane:DCM=9:1) to afford a off white solid. LCMS confirmed as the desired compound 2-bromo-4-(5-{3-chloro-4-[(1-methylethyl)oxy]phenyl}-1,2,4-oxadiazol-3-yl)-3-methylpyridine (0.54 g). δH (CDCl₃, 400 MHz): 1.49 (6H, d), 2.78 (3H, s), 4.73-4.74 (1H, m), 7.08 (1H, d), 7.87 (1H, d), 8.05-8.08 (1H, dd), 8.24 (1H, d), 8.38 (1H, d). MS (ES): C₁₇H₁₅BrClN₃O₂ requires 409; found 410.2 (M+H⁺).

### DESCRIPTION FOR D13

### ethyl 4-[4-(5-{3-chloro-4-[(1-methylethyl)oxy]phenyl}-1,2,4-oxadiazol-3-yl)-3-methyl-2-pyridinyl]butanoate (D13)

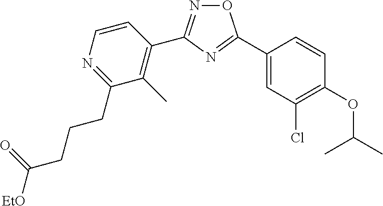

To a suspension of 2-bromo-4-(5-{3-chloro-4-[(1-methylethyl)oxy]phenyl}-1,2,4-oxadiazol-3-yl)-3-methylpyridine (130 mg), Pd(PPh₃)₄ (37 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 40 mins. Evaporate off the solvent, EtOAc (25 ml) was added. The organic phase was washed with 2M sodium hydroxide solution 25 mL (three times), dried over sodium sulphate and evaporated to afford ethyl 4-[4-(5-{3-chloro-4-[(1-methylethyl)oxy]phenyl}-1,2,4-oxadiazol-3-yl)-3-methyl-2-pyridinyl]butanoate (135 mg), applied to next step directly. MS (ES): C₂₃H₂₆ClN₃O₄ requires 444; found 445.1 (M+H⁺).

### DESCRIPTION FOR D14

### 3-bromo-2-chloro-N-hydroxybenzenecarboximidamide (D14)

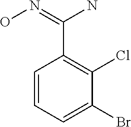

A mixture of 3-bromo-2-chlorobenzonitrile (2.0 g), sodium bicarbonate (1.55 g) and hydroxylamine hydrochloride (1.28 g) in ethanol (50 mL) was heated at reflux for overnight. The inorganic precipitate was filtered off. The solid was washed thoroughly with ethanol. The filtrate was concentrated. The obtained solid was dried in vacuo to afford 3-bromo-2-chloro-N-hydroxybenzenecarboximidamide (2.13 g). MS (ES): C₇H₆BrClN₂O requires 248; found 249.1 (M+H⁺).

### DESCRIPTION FOR D15

### 5-[3-(3-bromo-2-chlorophenyl)-1,2,4-oxadiazol-5-yl]-3-chloro-2-[(1-methylethyl)oxy]pyridine (D15)

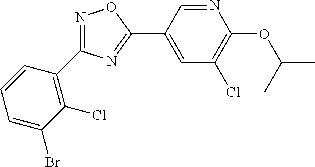

5-chloro-6-[(1-methylethyl)oxy]-3-pyridinecarboxylic acid (2.6 g), EDCI (4.62 g), HOBT (3.69 g) were dissolved in THF (50 ml). After the mixture was stirred for 10 mins at RT, a solution of 3-bromo-2-chloro-N-hydroxybenzenecarboximidamide (2.13 g) in THF (50 ml) was added slowly. The obtained mixture was stirred for further 2 hours at room temperature. LCMS show only one intermediate was found. After addition of TBAF (12.6 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (Hexane:DCM=10:1) to afford a off white solid. LCMS confirmed as the desired compound 5-[3-(3-bromo-2-chlorophenyl)-1,2,4-oxadiazol-5-yl]-3-chloro-2-[(1-methylethyl)oxy]pyridine (3.25 g). δH (CDCl₃, 400 MHz): 1.46 (6H, d), 5.49-5.52 (1H, m), 7.30 (1H, dd), 7.82-7.85 (1H, dd), 7.90-7.92 (1H, dd), 8.39 (1H, d), 8.88 (1H, d). MS (ES): C₁₆H₁₂BrCl₂N₃O₂ requires 427; found 428.2 (M+H⁺).

### DESCRIPTION FOR D16

### ethyl 3-[2-chloro-3-(5-{5-chloro-6-[(1-methylethyl)oxy]-3-pyridinyl}-1,2,4-oxadiazol-3-yl)phenyl]propanoate (D16)

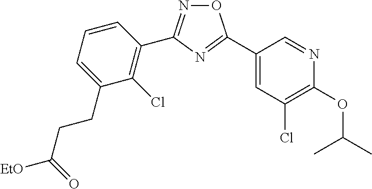

To a suspension of 5-[3-(3-bromo-2-chlorophenyl)-1,2,4-oxadiazol-5-yl]-3-chloro-2-[(1-methylethyl)oxy]pyridine (100 mg), Pd₂(dba)₃ (32 mg), P(o-tol)₃ (21 mg) in THF (3 ml) stirred under nitrogen at room temperature was added a solution of bromo[3-(ethyloxy)-3-oxopropyl]zinc (0.5 M in THF, 1.2 ml) in one portion. The reaction mixture was stirred at room temperature for 0.5 hours. Evaporate off the solvent, EtOAc (25 ml) was added. The organic phase was washed with 2M sodium hydroxide solution 25 mL (three times), dried over sodium sulphate and evaporated to afford ethyl 3-[2-chloro-3-(5-{5-chloro-6-[(1-methylethyl)oxy]-3-pyridinyl}-1,2,4-oxadiazol-3-yl)phenyl]propanoate (146 mg), applied to next step directly. MS (ES): C₂₁H₂₁Cl₂N₃O₄ requires 449; found 450.1 (M+H⁺).

### DESCRIPTION FOR D17

### ethyl 4-[2-chloro-3-(5-{5-chloro-6-[(1-methylethyl)oxy]-3-pyridinyl}-1,2,4-oxadiazol-3-yl)phenyl]butanoate (D17)

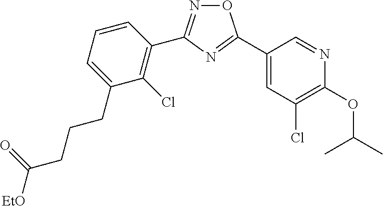

To a suspension of 5-[3-(3-bromo-2-chlorophenyl)-1,2,4-oxadiazol-5-yl]-3-chloro-2-[(1-methylethyl)oxy]pyridine (150 mg), Pd₂(dba)₃ (64 mg), tris(1,1-dimethylethyl)phosphane (40 mg), Cs₂CO₃ (45 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at 40° C. for 0.5 hours. The mixture was cooled to room temperature. Evaporate off the solvent, EtOAc (25 ml) was added. The organic phase was washed with 2M sodium hydroxide solution 25 mL (three times), dried over sodium sulphate and evaporated to afford ethyl 4-[2-chloro-3-(5-{5-chloro-6-[(1-methylethyl)oxy]-3-pyridinyl}-1,2,4-oxadiazol-3-yl)phenyl]butanoate (147 mg), applied to next step directly. MS (ES): C₂₂H₂₃Cl₂N₃O₄ requires 463; found 464.1 (M+H⁺).

### DESCRIPTION FOR D18

5-[3-(3-bromo-2-chlorophenyl)-1,2,4-oxadiazol-5-yl]-2-[(1-methylethyl)oxy]benzonitrile (D18)

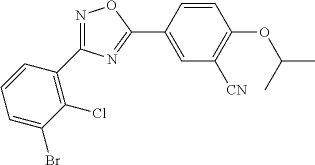

3-cyano-4-[(1-methylethyl)oxy]benzoic acid (1.05 g), EDCI (1.96 g), HOBT (1.57 g) were dissolved in THF (50 ml). After the mixture was stirred for 10 mins at RT, a solution of 3-bromo-N-hydroxy-2-methylbenzenecarboximidamide (1.2 g) in THF (10 ml) was added slowly. The obtained mixture was stirred for further 2 hours at room temperature. LCMS show only one intermediate was found. After addition of TBAF (4.87 g), the reaction mixture was heated to 80° C. for 2 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-[3-(3-bromo-2-chlorophenyl)-1,2,4-oxadiazol-5-yl]-2-[(1-methylethyl)oxy]benzonitrile (1.03 g). MS (ES): C₁₈H₁₃BrClN₃O₂ requires 417; found 418.2 (M+H⁺).

### DESCRIPTION FOR D19

### ethyl 3-[2-chloro-3-(5-{3-cyano-4-[(1-methylethyl)oxy]phenyl}-1,2,4-oxadiazol-3-yl)phenyl]propanoate (D19)

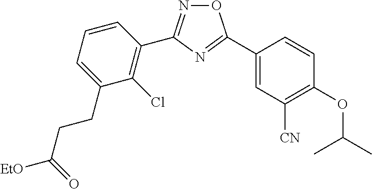

To a suspension of 5-[3-(3-bromo-2-chlorophenyl)-1,2,4-oxadiazol-5-yl]-2-[(1-methylethyl)oxy]benzonitrile (150 mg), Pd₂(dba)₃ (49 mg), tris(1,1-dimethylethyl)phosphane (31 mg), Cs₂CO₃ (35 mg) in THF (5 ml) stirred under nitrogen at room temperature was added a solution of bromo[3-(ethyloxy)-3-oxopropyl]zinc (0.5 M in THF, 2 ml) in one portion. The reaction mixture was stirred at room temperature for 1.5 hours. Evaporate off the solvent, EtOAc (25 ml) was added. The organic phase was washed with 2M sodium hydroxide solution 25 ml (three times), dried over sodium sulphate and evaporated to afford ethyl 3-[2-chloro-3-(5-{3-cyano-4-[(1-methylethyl)oxy]phenyl}-1,2,4-oxadiazol-3-yl)phenyl]propanoate (159 mg), applied to next step directly. MS (ES): C₂₃H₂₂ClN₃O₄ requires 439; found 440.1 (M+H⁺).

### DESCRIPTION FOR D20

### ethyl 4-[2-chloro-3-(5-{3-cyano-4-[(1-methylethyl)oxy]phenyl}-1,2,4-oxadiazol-3-yl)phenyl]butanoate (D20)

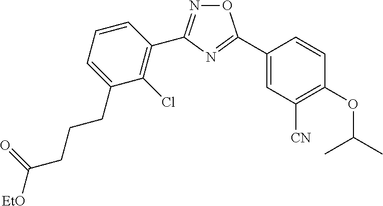

To a suspension of 5-[3-(3-bromo-2-chlorophenyl)-1,2,4-oxadiazol-5-yl]-2-[(1-methylethyl)oxy]benzonitrile (150 mg), Pd₂(dba)₃ (49 mg), tris(1,1-dimethylethyl)phosphane (31 mg), Cs₂CO₃ (35 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 2 hours. The mixture was cooled to room temperature. Evaporate off the solvent, EtOAc (25 ml) was added. The organic phase was washed with 2M sodium hydroxide solution 25 mL (three times), dried over sodium sulphate and evaporated to afford ethyl 4-[2-chloro-3-(5-{3-cyano-4-[(1-methylethyl)oxy]phenyl}-1,2,4-oxadiazol-3-yl)phenyl]butanoate (142 mg), applied to next step directly. MS (ES): C₂₄H₂₄ClN₃O₄ requires 453; found 454.1 (M+H⁺).

### DESCRIPTION FOR D21

### ethyl 6-[2-chloro-3-(5-{3-cyano-4-[(1-methylethyl)oxy]phenyl}-1,2,4-oxadiazol-3-yl)phenyl]hexanoate (D21)

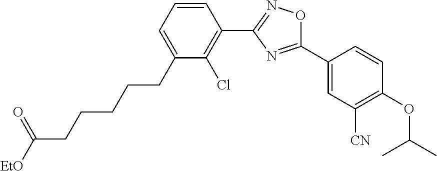

To a suspension of 5-[3-(3-bromo-2-chlorophenyl)-1,2,4-oxadiazol-5-yl]-2-[(1-methylethyl)oxy]benzonitrile (150 mg), Pd₂(dba)₃ (49 mg), tris(1,1-dimethylethyl)phosphane (31 mg), Cs₂CO₃ (35 mg) in THF (5 ml) stirred under nitrogen at room temperature was added a solution of bromo[6-(ethyloxy)-6-oxohexyl]zinc (0.5 M in THF, 2 ml) in one portion. The reaction mixture was stirred at room temperature for 2 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 6-[2-chloro-3-(5-{3-cyano-4-[(1-methylethyl)oxy]phenyl}-1,2,4-oxadiazol-3-yl)phenyl]hexanoate (167 mg), applied to next step directly. MS (ES): C₂₆H₂₈ClN₃O₄ requires 481; found 482.1 (M+H⁺).

### DESCRIPTION FOR D22

### 3-bromo-2-ethyl-N-hydroxybenzenecarboximidamide (D22)

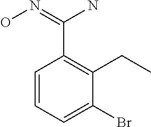

A mixture of 3-bromo-2-ethylbenzonitrile (1.5 g), sodium bicarbonate (1.20 g) and hydroxylamine hydrochloride (1.0 g) in ethanol (65 ml) was heated at reflux for overnight. The inorganic precipitate was filtered off. The solid was washed thoroughly with ethanol. The filtrate was concentrated. The obtained solid was dried in vacuo to afford 3-bromo-2-ethyl-N-hydroxybenzenecarboximidamide (1.62 g). MS (ES): C₉H₁₁BrN₂O requires 242; found 243.1 (M+H⁺).

### DESCRIPTION FOR D23

### 5-[3-(3-bromo-2-ethylphenyl)-1,2,4-oxadiazol-5-yl]-3-chloro-2-[(1-methylethyl)oxy]pyridine (D23)

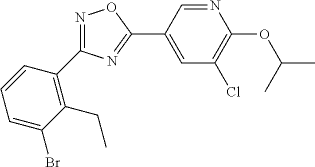

5-chloro-6-[(1-methylethyl)oxy]-3-pyridinecarboxylic acid (2.0 g), EDCI (3.56 g), HOBT (2.84 g) were dissolved in THF (50 ml). After the mixture was stirred for 10 mins at RT, a solution of 3-bromo-2-ethyl-N-hydroxybenzenecarboximidamide (1.62 g) in THF (50 ml) was added slowly. The obtained mixture was stirred for further 2 hours at room temperature. LCMS show only one intermediate was found. After addition of TBAF (12.6 g), the reaction mixture was heated to 80° C. for 2 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (Hexane:DCM=10:1) to afford a off white solid. LCMS confirmed as the desired compound 5-[3-(3-bromo-2-ethylphenyl)-1,2,4-oxadiazol-5-yl]-3-chloro-2-[(1-methylethyl)oxy]pyridine (2.41 g). δH (CDCl₃, 400 MHz): 1.28 (3H, t), 1.46 (611, d), 3.13-3.17 (2H, m), 5.48-5.52 (1H, m), 7.18-7.22 (1H, dd), 7.73-7.75 (1H, dd), 7.87-7.89 (1H, dd), 8.38 (1H, d), 8.88 (1H, d). MS (ES): C₁₈H₁₇BrClN₃O₂ requires 421; found 422.2 (M+H⁺).

### DESCRIPTION FOR D24

### ethyl 3-[3-(5-{5-chloro-6-[(1-methylethyl)oxy]-3-pyridinyl}-1,2,4-oxadiazol-3-yl)-2-ethylphenyl]propanoate (D24)

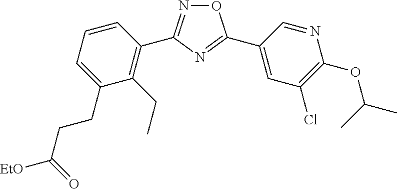

To a suspension of 5-[3-(3-bromo-2-ethylphenyl)-1,2,4-oxadiazol-5-yl]-3-chloro-2-[(1-methylethyl)oxy]pyridine (120 mg), Pd₂(dba)₃ (39 mg), P(o-tol)₃ (26 mg) in THF (3 ml) stirred under nitrogen at room temperature was added a solution of bromo[3-(ethyloxy)-3-oxopropyl]zinc (0.5 M in THF, 1.7 ml) in one portion. The reaction mixture was stirred at room temperature for overnight. Evaporate off the solvent, EtOAc (25 ml) was added. The organic phase was washed with 2M sodium hydroxide solution 25 ml (three times), dried over sodium sulphate and evaporated to afford ethyl 3-[3-(5-{5-chloro-6-[(1-methylethyl)oxy]-3-pyridinyl}-1,2,4-oxadiazol-3-yl)-2-ethylphenyl]propanoate (108 mg), applied to next step directly. MS (ES): C₂₃H₂₆ClN₃O₄ requires 443; found 444.1 (M+H⁺).

### DESCRIPTION FOR D25

### ethyl 4-[3-(5-{5-chloro-6-[(1-methylethyl)oxy]-3-pyridinyl}-1,2,4-oxadiazol-3-yl)-2-ethylphenyl]butanoate (D25)

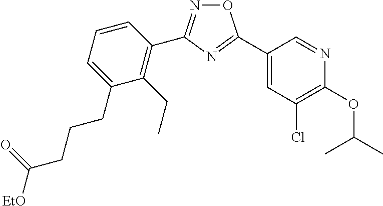

To a suspension of 5-[3-(3-bromo-2-ethylphenyl)-1,2,4-oxadiazol-5-yl]-3-chloro-2-[(1-methylethyl)oxy]pyridine (180 mg), Pd₂(dba)₃ (58 mg), P(o-tol)₃ (39 mg) in THF (5 ml) stirred under nitrogen at room temperature was added a solution of bromo[4-(ethyloxy)-4-oxobutyl]zinc (0.5 M in THF, 2.5 ml) in one portion. The reaction mixture was stirred at 40° C. for overnight. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{5-chloro-6-[(1-methylethyl)oxy]-3-pyridinyl}-1,2,4-oxadiazol-3-yl)-2-ethylphenyl]butanoate (174 mg), applied to next step directly. MS (ES): C₂₄H₂₈ClN₃O₄ requires 457; found 458.1 (M+H⁺).

### DESCRIPTION FOR D26

### 5-[3-(3-bromo-2-ethylphenyl)-1,2,4-oxadiazol-5-yl]-2-[(1-methylethyl)oxy]benzonitrile (D26)

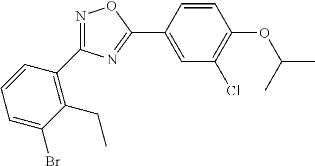

3-cyano-4-[(1-methylethyl)oxy]benzoic acid (0.91 g), EDCI (1.70 g), HOBT (1.36 g) were dissolved in THF (50 ml). After the mixture was stirred for 10 mins at RT, a solution of 3-bromo-2-ethyl-N-hydroxybenzenecarboximidamide (0.98 g) in THF (10 ml) was added slowly. The obtained mixture was stirred for further 2 hours at room temperature. LCMS show only one intermediate was found. After addition of TBAF (4.22 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-[3-(3-bromo-2-ethylphenyl)-1,2,4-oxadiazol-5-yl]-2-[(1-methylethyl)oxy]benzonitrile (0.65 g). MS (ES): C₂₀H₁₈BrN₃O₂ requires 411; found 412.2 (M+H⁺).

### DESCRIPTION FOR D27

### ethyl 4-[3-(5-{3-cyano-4-[(1-methylethyl)oxy]phenyl}-1,2,4-oxadiazol-3-yl)-2-ethylphenyl]butanoate (D27)

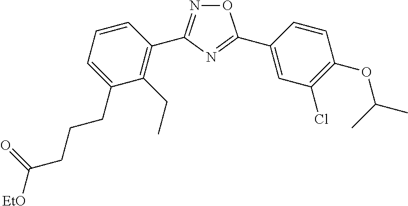

To a suspension of 5-[3-(3-bromo-2-ethylphenyl)-1,2,4-oxadiazol-5-yl]-2-[(1-methylethyl)oxy]benzonitrile (150 mg), Pd₂(dba)₃ (50 mg), tris(1,1-dimethylethyl)phosphane (31 mg), Cs₂CO₃ (35 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for overnight. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-cyano-4-[(1-methylethyl)oxy]phenyl}-1,2,4-oxadiazol-3-yl)-2-ethylphenyl]butanoate (152 mg), applied to next step directly. MS (ES): C₂₆H₂₉N₃O₄ requires 447; found 448.1 (M+H⁺).

### DESCRIPTION FOR D28

### 3-bromo-N-hydroxy-2-(methyloxy)benzenecarboximidamide (D28)

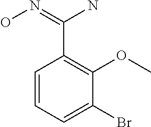

A mixture of 3-bromo-2-(methyloxy)benzonitrile (1.3 g), sodium bicarbonate (1.03 g) and hydroxylamine hydrochloride (0.85 g) in ethanol (50 ml) was heated at reflux for overnight. The inorganic precipitate was filtered off. The solid was washed thoroughly with ethanol. The filtrate was concentrated. The obtained solid was dried in vacuo to afford 3-bromo-N-hydroxy-2-(methyloxy)benzenecarboximidamide (1.37 g). MS (ES): C₈H₉BrN₂O₂ requires 244; found 245.1 (M+H⁺).

### DESCRIPTION FOR D29

### 3-[3-bromo-2-(methyloxy)phenyl]-5-{3-chloro-4-[(1-methylethyl)oxy]phenyl}-1,2,4-oxadiazole (D29)

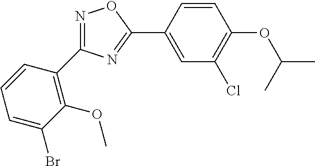

3-Chloro-4-[(1-methylethyl)oxy]benzoic acid (1.63 g), EDCI (2.90 g), HOBT (2.32 g) were dissolved in THF (50 ml). After the mixture was stirred for 10 mins at RT, a solution of 3-bromo-N-hydroxy-2-(methyloxy)benzenecarboximidamide (1.37 g) in THF (50 ml) was added slowly. The obtained mixture was stirred for further 2 hours at room temperature. LCMS show only one intermediate was found. After addition of TBAF (1.98 g), the reaction mixture was heated to 80° C. for 4 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (Hexane:DCM=9:1) to afford a off white solid. LCMS confirmed as the desired compound 3-[3-bromo-2-(methyloxy)phenyl]-5-{3-chloro-4-[(1-methylethyl)oxy]phenyl}-1,2,4-oxadiazole (2.53 g). MS (ES): C₁₈H₁₆BrClN₂O₃ requires 422; found 423.2 (M+H⁺).

### DESCRIPTION FOR D30

### ethyl 3-[3-(5-{3-chloro-4-[(1-methylethyl)oxy]phenyl}-1,2,4-oxadiazol-3-yl)-2-(methyloxy)phenyl]propanoate (D30)

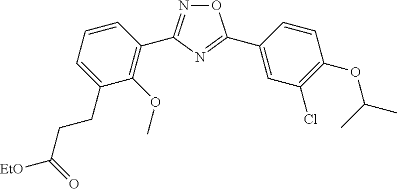

To a suspension of 3-[3-bromo-2-(methyloxy)phenyl]-5-{3-chloro-4-[(1-methylethyl)oxy]phenyl}-1,2,4-oxadiazole (150 mg), Pd₂(dba)₃ (49 mg), P(o-tol)₃ (32 mg) in THF (5 ml) stirred under nitrogen at room temperature was added a solution of bromo[3-(ethyloxy)-3-oxopropyl]zinc (0.5 M in THF, 2 ml) in one portion. The reaction mixture was stirred at 40° C. for 2 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 3-[3-(5-{3-chloro-4-[(1-methylethyl)oxy]phenyl}-1,2,4-oxadiazol-3-yl)-2-(methyloxy)phenyl]propanoate (153 mg), applied to next step directly. MS (ES): C₂₂H₂₄ClN₃O₅ requires 445; found 446.1 (M+H⁺).

### DESCRIPTION FOR D31

### ethyl 4-[3-(5-{3-chloro-4-[(1-methylethyl)oxy]phenyl}-1,2,4-oxadiazol-3-yl)-2-(methyloxy)phenyl]butanoate (D31)

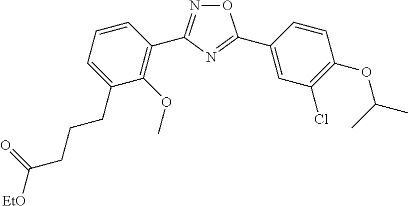

To a suspension of 3-[3-bromo-2-(methyloxy)phenyl]-5-{3-chloro-4-[(1-methylethyl)oxy]phenyl}-1,2,4-oxadiazole (150 mg), Pd₂(dba)₃ (48 mg), P(o-tol)₃(32 mg) in THF (5 ml) stirred under nitrogen at room temperature was added a solution of bromo[4-(ethyloxy)-4-oxobutyl]zinc (0.5 M in THF, 2 mL) in one portion. The reaction mixture was stirred at 40° C. for overnight. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-chloro-4-[(1-methylethyl)oxy]phenyl}-1,2,4-oxadiazol-3-yl)-2-(methyloxy)phenyl]butanoate (160 mg), applied to next step directly. MS (ES): C₂₄H₂₇ClN₂O₅ requires 459; found 460.1 (M+H⁺).

### DESCRIPTION FOR D32

### 5-{3-[3-bromo-2-(methyloxy)phenyl]-1,2,4-oxadiazol-5-yl}-2-[(1-methylethyl)oxy]benzonitrile (D32)

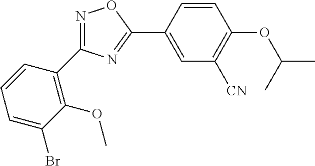

3-cyano-4-[(1-methylethyl)oxy]benzoic acid (0.8 g), EDCI (1.49 g), HOBT (1.19 g) were dissolved in THF (10 ml). After the mixture was stirred for 10 mins at RT, a solution of 3-bromo-N-hydroxy-2-(methyloxy)benzenecarboximidamide (1.1 g) in THF (10 ml) was added slowly. The obtained mixture was stirred for further 2 hours at room temperature. LCMS show only one intermediate was found. After addition of TBAF (4.87 g), the reaction mixture was heated to 80° C. for 2 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=9:1) to afford a off white solid. LCMS confirmed as the desired compound 5-{3-[3-bromo-2-(methyloxy)phenyl]-1,2,4-oxadiazol-5-yl}-2-[(1-methylethyl)oxy]benzonitrile (1.03 g). MS (ES): C₁₉H₁₆BrN₃O₃ requires 413; found 414.2 (M+H⁺).

### DESCRIPTION FOR D33

### ethyl 3-[3-(5-{3-cyano-4-[(1-methylethyl)oxy]phenyl}-1,2,4-oxadiazol-3-yl)-2-(methyloxy)phenyl]propanoate (D33)

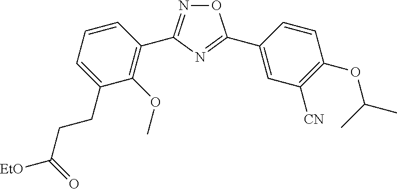

To a suspension of 5-{3-[3-bromo-2-(methyloxy)phenyl]-1,2,4-oxadiazol-5-yl}-2-[(1-methylethyl)oxy]benzonitrile (150 mg), Pd₂(dba)₃ (52 mg), tris(1,1-dimethylethyl)phosphane (33 mg), Cs₂CO₃ (37 mg) in THF (5 mL) stirred under nitrogen at room temperature was added a solution of bromo[3-(ethyloxy)-3-oxopropyl]zinc (0.5 M in THF, 2 ml) in one portion. The reaction mixture was stirred at room temperature for 2 hours. Evaporate off the solvent, EtOAc (25 ml) was added. The organic phase was washed with 2M sodium hydroxide solution 25 ml (three times), dried over sodium sulphate and evaporated to afford ethyl 3-[3-(5-{3-cyano-4-[(1-methylethyl)oxy]phenyl}-1,2,4-oxadiazol-3-yl)-2-(methyloxy)phenyl]propanoate (147 mg), applied to next step directly. MS (ES): C₂₄H₂₅N₃O₅ requires 435; found 436.1 (M+H⁺).

### DESCRIPTION FOR D34

### ethyl 4-[3-(5-{3-cyano-4-[(1-methylethyl)oxy]phenyl}-1,2,4-oxadiazol-3-yl)-2-(methyloxy)phenyl]butanoate (D34)

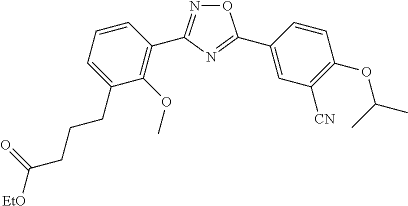

To a suspension of 5-{3-[3-bromo-2-(methyloxy)phenyl]-1,2,4-oxadiazol-5-yl}-2-[(1-methylethyl)oxy]benzonitrile (150 mg), Pd₂(dba)₃ (49 mg), tris(1,1-dimethylethyl)phosphane (31 mg), Cs₂CO₃ (35 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 2 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-cyano-4-[(1-methylethyl)oxy]phenyl}-1,2,4-oxadiazol-3-yl)-2-(methyloxy)phenyl]butanoate (139 mg), applied to next step directly. MS (ES): C₂₅H₂₇N₃O₅ requires 449; found 450.1 (M+H⁺).

### DESCRIPTION FOR D35

### 2-bromo-N-hydroxy-4-pyridinecarboximidamide (D35)

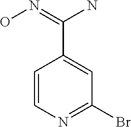

A mixture of 2-bromo-4-pyridinecarbonitrile (0.7 g), sodium bicarbonate (0.65 g) and hydroxylamine hydrochloride (0.53 g) in ethanol (50 ml) was heated at reflux for overnight. The inorganic precipitate was filtered off. The solid was washed thoroughly with ethanol. The filtrate was concentrated. The obtained solid was dried in vacuo to afford 2-bromo-N-hydroxy-4-pyridinecarboximidamide (0.78 g). MS (ES): C₆H₆BrN₃O requires 215; found 216.1 (M+H⁺).

### DESCRIPTION FOR D36

### 5-[3-(2-bromo-4-pyridinyl)-1,2,4-oxadiazol-5-yl]-2-[(1-methylethyl)oxy]benzonitrile (D36)

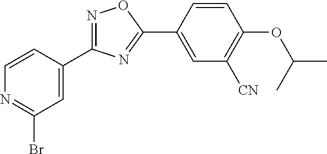

3-cyano-4-[(1-methylethyl)oxy]benzoic acid (0.8 g), EDCI (1.49 g), HOBT (1.19 g) were dissolved in THF (10 ml). After the mixture was stirred for 10 mins at RT, a solution of 2-bromo-N-hydroxy-4-pyridinecarboximidamide (0.78 g) in THF (10 ml) was added slowly. The obtained mixture was stirred for further 2 hours at room temperature. LCMS show only one intermediate was found. After addition of TBAF (4.22 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=8:1) to afford a off white solid. LCMS confirmed as the desired compound 5-[3-(2-bromo-4-pyridinyl)-1,2,4-oxadiazol-5-yl]-2-[(1-methylethyl)oxy]benzonitrile (0.95 g). MS (ES): C₁₇H₁₃BrN₄O₂ requires 384; found 385.2 (M+H⁺).

### DESCRIPTION FOR D37

### ethyl 3-[4-(5-{3-cyano-4-[(1-methylethyl)oxy]phenyl}-1,2,4-oxadiazol-3-yl)-2-pyridinyl]propanoate (D37)

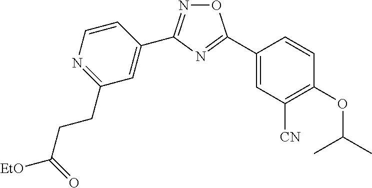

To a suspension of 5-[3-(2-bromo-4-pyridinyl)-1,2,4-ox1adiazol-5-yl]-2-[(1-methylethyl)oxy]benzonitrile (150 mg), Pd₂(dba)₃ (50 mg), tris(1,1-dimethylethyl)phosphane (106 mg), Cs₂CO₃ (35 mg) in THF (5 ml) stirred under nitrogen at room temperature was added a solution of bromo[3-(ethyloxy)-3-oxopropyl]zinc (0.5 M in THF, 2 ml) in one portion. The reaction mixture was stirred at room temperature for 1.5 hours. Evaporate off the solvent, EtOAc (25 ml) was added. The organic phase was washed with 2M sodium hydroxide solution 25 ml (three times), dried over sodium sulphate and evaporated to afford ethyl 3-[4-(5-{3-cyano-4-[(1-methylethyl)oxy]phenyl}-1,2,4-oxadiazol-3-yl)-2-pyridinyl]propanoate (158 mg), applied to next step directly. MS (ES): C₂₂H₂₂N₄O₄ requires 406; found 407.1 (M+H⁺).

### DESCRIPTION FOR D38

### ethyl 4-[4-(5-{3-cyano-4-[(1-methylethyl)oxy]phenyl}-1,2,4-oxadiazol-3-yl)-2-pyridinyl]butanoate (D38)

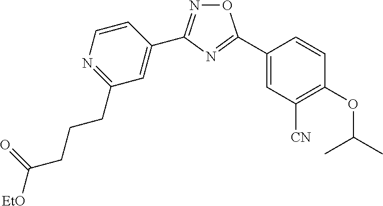

To a suspension of 5-[3-(2-bromo-4-pyridinyl)-1,2,4-oxadiazol-5-yl]-2-[(1-methylethyl)oxy]benzonitrile (150 mg), Pd₂(dba)₃ (52 mg), tris(1,1-dimethylethyl)phosphane (33 mg), Cs₂CO₃ (37 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 1.5 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[4-(5-{3-cyano-4-[(1-methylethyl)oxy]phenyl}-1,2,4-oxadiazol-3-yl)-2-pyridinyl]butanoate (139 mg), applied to next step directly. MS (ES): C₂₃H₂₄N₄O₄ requires 420; found 421.1 (M+H⁺).

### DESCRIPTION FOR D39

### 3-bromo-4-fluoro-N-hydroxybenzenecarboximidamide (D39)

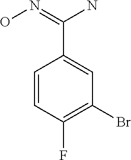

A mixture of 3-bromo-4-fluorobenzonitrile (2 g), sodium bicarbonate (1.68 g) and hydroxylamine hydrochloride (1.39 g) in ethanol (50 ml) was heated at reflux for overnight. The inorganic precipitate was filtered off. The solid was washed thoroughly with ethanol. The filtrate was concentrated. The obtained solid was dried in vacuo to afford 3-bromo-4-fluoro-N-hydroxybenzenecarboximidamide (2.02 g). MS (ES): C₇H₆BrFN₂O requires 232; found 233.1 (M+H⁺).

### DESCRIPTION FOR D40

### 5-[3-(3-bromo-4-fluorophenyl)-1,2,4-oxadiazol-5-yl]-2-[(1-methylethyl)oxy]benzonitrile (D40)

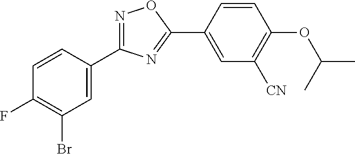

3-cyano-4-[(1-methylethyl)oxy]benzoic acid (1.6 g), EDCI (3.0 g), HOBT (2.4 g) were dissolved in THF (10 ml). After the mixture was stirred for 10 mins at RT, a solution of 2-bromo-N-hydroxy-4-pyridinecarboximidamide (2.2 g) in THF (10 ml) was added slowly. The obtained mixture was stirred for further 2 hours at room temperature. LCMS show only one intermediate was found. After addition of TBAF (9.66 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-[3-(3-bromo-4-fluorophenyl)-1,2,4-oxadiazol-5-yl]-2-[(1-methylethyl)oxy]benzonitrile (1.1 g). MS (ES): C₁₈H₁₃BrFN₃O₂ requires 401; found 402.2 (M+H⁺).

### DESCRIPTION FOR D41

### ethyl 3-[5-(5-{3-cyano-4-[(1-methylethyl)oxy]phenyl}-1,2,4-oxadiazol-3-yl)-2-fluorophenyl]propanoate (D41)

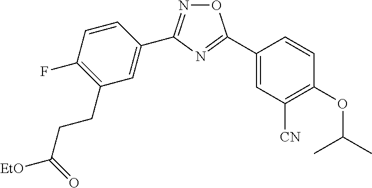

To a suspension of 5-[3-(3-bromo-4-fluorophenyl)-1,2,4-oxadiazol-5-yl]-2-[(1-methylethyl)oxy]benzonitrile (150 mg), Pd₂(dba)₃ (51 mg), tris(1,1-dimethylethyl)phosphane (92 mg), Cs₂CO₃ (36 mg) in THF (7 ml) stirred under nitrogen at room temperature was added a solution of bromo[3-(ethyloxy)-3-oxopropyl]zinc (0.5 M in THF, 2 ml) in one portion. The reaction mixture was stirred at room temperature for 1.5 hours. Evaporate off the solvent, EtOAc (25 ml) was added. The organic phase was washed with 2M sodium hydroxide solution 25 ml (three times), dried over sodium sulphate and evaporated to afford ethyl 3-[5-(5-{3-cyano-4-[(1-methylethyl)oxy]phenyl}-1,2,4-oxadiazol-3-yl)-2-fluorophenyl]propanoate (168 mg), applied to next step directly. MS (ES): C₂₃H₂₂FN₃O₄ requires 423; found 424.1 (M+H⁺).

### DESCRIPTION FOR D42

### ethyl 4-[5-(5-{3-cyano-4-[(1-methylethyl)oxy]phenyl}-1,2,4-oxadiazol-3-yl)-2-fluorophenyl]butanoate (D42)

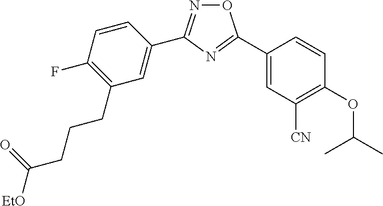

To a suspension of 5-[3-(3-bromo-4-fluorophenyl)-1,2,4-oxadiazol-5-yl]-2-[(1-methylethyl)oxy]benzonitrile (150 mg), Pd₂(dba)₃ (52 mg), tris(1,1-dimethylethyl)phosphane (36 mg), Cs₂CO₃ (37 mg) in THF (5 ml) stirred under nitrogen at room temperature was added a solution of bromo[4-(ethyloxy)-4-oxobutyl]zinc (0.5 M in THF, 2 ml) in one portion. The reaction mixture was stirred at room temperature for 1.5 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[5-(5-{3-cyano-4-[(1-methylethyl)oxy]phenyl}-1,2,4-oxadiazol-3-yl)-2-fluorophenyl]butanoate (168 mg), applied to next step directly. MS (ES): C₂₄H₂₄FN₃O₄ requires 437; found 437.1 (M+H⁺).

### DESCRIPTION FOR D43

### 3-bromo-N-hydroxy-2-(trifluoromethyl)benzenecarboximidamide (D43)

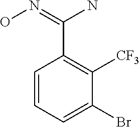

A mixture of 3-bromo-2-(trifluoromethyl)benzonitrile (0.96 g), sodium bicarbonate (0.65 g) and hydroxylamine hydrochloride (0.54 g) in ethanol (30 ml) was heated at reflux for overnight. The inorganic precipitate was filtered off. The solid was washed thoroughly with ethanol. The filtrate was concentrated. The obtained solid was dried in vacuo to afford 3-bromo-N-hydroxy-2-(trifluoromethyl)benzenecarboximidamide (1.06 g). MS (ES): C₈H₆BrF₃N₂O requires 282; found 283.1 (M+H⁺).

### DESCRIPTION FOR D44

### 5-{3-[3-bromo-2-(trifluoromethyl)phenyl]-1,2,4-oxadiazol-5-yl}-2-[(1-methylethyl)oxy]benzonitrile (D44)

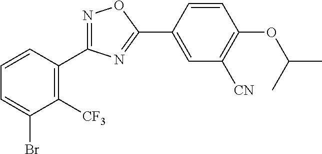

3-cyano-4-[(1-methylethyl)oxy]benzoic acid (0.66 g), EDCI (1.24 g), HOBT (1 g) were dissolved in THF (10 ml). After the mixture was stirred for 10 mins at RT, a solution of 3-bromo-N-hydroxy-2-(trifluoromethyl)benzenecarboximidamide (1.06 g) in THF (10 ml) was added slowly. The obtained mixture was stirred for further 2 hours at room temperature. LCMS show only one intermediate was found. After addition of TBAF (4.19 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-{3-[3-bromo-2-(trifluoromethyl)phenyl]-1,2,4-oxadiazol-5-yl}-2-[(1-methylethyl)oxy]benzonitrile (0.62 g). MS (ES): C₁₉H₁₃BrF₃N₃O₂ requires 451; found 452.2 (M+H⁺).

### DESCRIPTION FOR D45

### ethyl 3-[3-(5-{3-cyano-4-[(1-methylethyl)oxy]phenyl}-1,2,4-oxadiazol-3-yl)-2-(trifluoromethyl)phenyl]propanoate (D45)

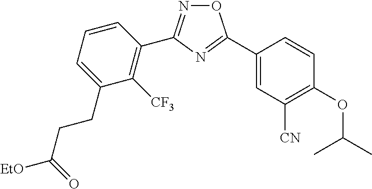

To a suspension of 5-{3-[3-bromo-2-(trifluoromethyl)phenyl]-1,2,4-oxadiazol-5-yl}-2-[(1-methylethyl)oxy]benzonitrile (115 mg), Pd₂(dba)₃ (35 mg), tris(1,1-dimethylethyl)phosphane (22 mg), Cs₂CO₃ (25 mg) in THF (7 ml) stirred under nitrogen at room temperature was added a solution of bromo[3-(ethyloxy)-3-oxopropyl]zinc (0.5 M in THF, 1.5 ml) in one portion. The reaction mixture was stirred at room temperature for 2 hours. Evaporate off the solvent, EtOAc (25 ml) was added. The organic phase was washed with 2M sodium hydroxide solution 25 ml (three times), dried over sodium sulphate and evaporated to afford ethyl 3-[3-(5-{3-cyano-4-[(1-methylethyl)oxy]phenyl}-1,2,4-oxadiazol-3-yl)-2-(trifluoromethyl)phenyl]propanoate (121 mg), applied to next step directly. MS (ES): C₂₄H₂₃F₃N₄O₄ requires 488; found 489.1 (M+H⁺).

### DESCRIPTION FOR D46

### ethyl 4-[3-(5-{3-cyano-4-[(1-methylethyl)oxy]phenyl}-1,2,4-oxadiazol-3-O-2-(trifluoromethyl)phenyl]butanoate (D46)

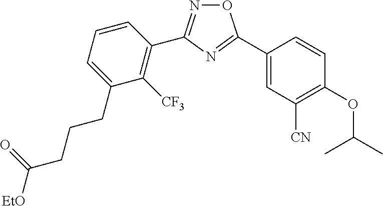

To a suspension of 5-{3-[3-bromo-2-(trifluoromethyl)phenyl]-1,2,4-oxadiazol-5-yl}-2-[(1-methylethyl)oxy]benzonitrile (115 mg), Pd₂(dba)₃ (35 mg), tris(1,1-dimethylethyl)phosphane (15 mg), Cs₂CO₃ (25 mg) in THF (7 ml) stirred under nitrogen at room temperature was added a solution of bromo[4-(ethyloxy)-4-oxobutyl]zinc (0.5 M in THF, 1.5 ml) in one portion. The reaction mixture was stirred at room temperature for 1.5 hours. The mixture was cooled to room temperature. Evaporate off the solvent, EtOAc (25 ml) was added. The organic phase was washed with 2M sodium hydroxide solution 25 ml (three times), dried over sodium sulphate and evaporated to afford ethyl 4-[3-(5-{3-cyano-4-[(1-methylethyl)oxy]phenyl}-1,2,4-oxadiazol-3-yl)-2-(trifluoromethyl)phenyl]butanoate (108 mg), applied to next step directly. MS (ES): C₂₄H₂₃F₃N₄O₄ requires 488; found 489.1 (M+H⁺).

### DESCRIPTION FOR D47

### N-hydroxy-3-(hydroxymethyl)-2-methylbenzenecarboximidamide (D47)

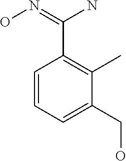

A mixture of 3-(hydroxymethyl)-2-methylbenzonitrile (2.0 g), sodium bicarbonate (3.42 g) and hydroxylamine hydrochloride (2.83 g) in ethanol (40 ml) was heated at reflux for overnight. The inorganic precipitate was filtered off. The solid was washed thoroughly with ethanol. The filtrate was concentrated. The obtained solid was dried in vacuo to afford N-hydroxy-3-(hydroxymethyl)-2-methylbenzenecarboximidamide (2.08 g). MS (ES): C₉H₁₂N₂O₂ requires 180; found 181.1 (M+H⁺).

### DESCRIPTION FOR D48

### 5-{3-[3-(hydroxymethyl)-2-methylphenyl]-1,2,4-oxadiazol-5-yl}-2-[(1-methylethyl)oxy]benzonitrile (D48)

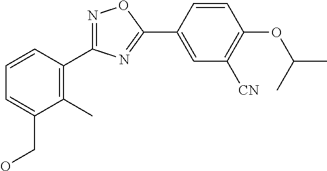

3-cyano-4-[(1-methylethyl)oxy]benzoic acid (1.32 g), EDCI (2.4 g), HOBT (2 g) were dissolved in THF (50 ml). After the mixture was stirred for 10 mins at Room Temperature, LCMS show one intermediate was formed. Evaporate off the solvent, EtOAc (50 ml) was added, organic layer was washed by water (50 ml) 3 times, combine the organic layer, evaporate off the solvent, THF (50 ml) readded, in the resulting solution a solution of 5-bromo-2-fluoro-N-hydroxybenzenecarboximidamide (0.73 g) in THF (10 ml) was added slowly. The obtained mixture was stirred for further 2 hours at room temperature. LCMS show only one intermediate was found. After addition of TBAF (4.19 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-{3-[3-(hydroxymethyl)-2-methylphenyl]-1,2,4-oxadiazol-5-yl}-2-[(1-methylethyl)oxy]benzonitrile (1.05 g). δH (CDCl₃, 400 MHz): 1.48 (6H, d), 2.62 (3H, s), 4.11-4.16 (1H, m), 4.79-4.84 (1H, m), 7.13 (1H, d), 7.35-7.39 (1H, dd), 7.58 (1H, d), 7.89 (1H, d), 8.34-8.36 (1H, dd), 8.44 (1H, d). MS (ES): C₂₀H₁₉N₃O₃ requires 349; found 350.2 (M+H⁺).

### DESCRIPTION FOR D49

### 5-[3-(3-formyl-2-methylphenyl)-1,2,4-oxadiazol-5-yl]-2-[(1-methylethyl)oxy]benzonitrile (D49)

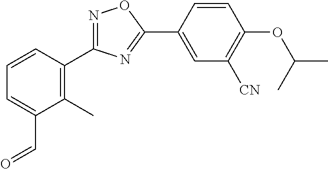

5-{3-[3-(hydroxymethyl)-2-methylphenyl]-1,2,4-oxadiazol-5-yl}-2-[(1-methylethyl)oxy]benzonitrile (66 mg) were well dissolved in DCM (3 ml). In the resulting solution was added Dess-Martin periodinane (99 mg), stirred for 1 hour, LCMS show complete conversion, filter off the solid, evaporate off the solvent. LCMS confirmed as the desired compound 5-[3-(3-formyl-2-methylphenyl)-1,2,4-oxadiazol-5-yl]-2-[(1-methylethyl)oxy]benzonitrile (64 mg). MS (ES): C₂₀H₁₇N₃O₃ requires 347; found 348.2 (M+H⁺).

### DESCRIPTION FOR D50

N,3-dihydroxy-2-methylbenzenecarboximidamide (D50)

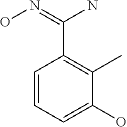

A mixture of 3-hydroxy-2-methylbenzonitrile (0.9 g), sodium bicarbonate (0.78 g) and hydroxylamine hydrochloride (0.64 g) in ethanol (40 ml) was heated at reflux for overnight. The inorganic precipitate was filtered off. The solid was washed thoroughly with ethanol. The filtrate was concentrated. The obtained solid was dried in vacuo to afford N,3-dihydroxy-2-methylbenzenecarboximidamide (0.71 g). MS (ES): C₁₀H₁₂N₂O₃ requires 208; found 209.1 (M+H⁺).

### DESCRIPTION FOR D51

### 3-(5-{5-chloro-6-[(1-methylethyl)oxy]-3-pyridinyl}-1,2,4-oxadiazol-3-yl)-2-methylphenol (D51)

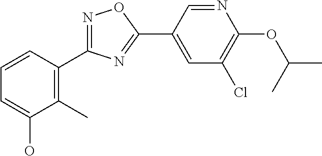

5-chloro-6-[(1-methylethyl)oxy]-3-pyridinecarboxylic acid (1.5 g), EDCI (2.67 g), HOBT (2.13 g) were dissolved in THF (50 ml). After the mixture was stirred for 10 mins at Room Temperature, LCMS show one intermediate was formed. Evaporate off the solvent, EtOAc (50 ml) was added, organic layer was washed by water (50 ml) 3 times, combine the organic layer, evaporate off the solvent, THF (50 ml) readded, in the resulting solution a solution of N,3-dihydroxy-2-methylbenzenecarboximidamide (0.71 g) in THF (10 ml) was added slowly. The obtained mixture was stirred for further 2 hours at room temperature. LCMS show only one intermediate was found. After addition of TBAF (7.28 g), the reaction mixture was heated to 80° C. for 3 days. The reaction was cooled down to room temperature, Evaporate off the solvent and added with EtOAc (100 ml). The organic solution was washed with saturated aqueous sodium dicarbonate solution (twice) and water (twice). After removal of the solvent, residue was purified by flash column (PE:EA=5:1) to afford a off white solid. LCMS confirmed as the desired compound 5-{3-[3-(hydroxymethyl)-2-methylphenyl]-1,2,4-oxadiazol-5-yl}-2-[(1-methylethyl)oxy]benzonitrile (0.49 g). δH (CDCl₃, 400 MHz): 1.48 (6H, d), 2.55 (3H, s), 5.48-5.51 (1H, m), 6.97 (1H, d), 7.21-7.25 (1H, dd), 7.58 (1H, d), 8.40 (1H, d), 8.88 (1H, d). MS (ES): C₁₇H₁₆ClN₃O₃ requires 345; found 346.2 (M+H⁺).

### DESCRIPTION FOR D52

### ethyl {[3-(5-{5-chloro-6-[(1-methylethyl)oxy]-3-pyridinyl}-1,2,4-oxadiazol-3-yl)-2-methylphenyl]oxy}acetate (D52)

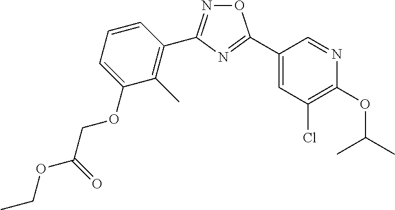

To a solution of 3-(5-{5-chloro-6-[(1-methylethyl)oxy]-3-pyridinyl}-1,2,4-oxadiazol-3-yl)-2-methylphenol (45 mg), PPh₃ (109 mg) and DIAD (84 mg) in Tetrahydrofuran (THF) (4 ml) stirred under nitrogen at room temp, in the resulting solution was added a solution of ethyl hydroxyacetate (29 mg) in one charge during 1 min. The reaction mixture was stirred at 22° C. for 1 hour. LCMS show complete conversion. Evaporate off the solvent, afford the desired compound ethyl {[3-(5-{5-chloro-6-[(1-methylethyl)oxy]-3-pyridinyl}-1,2,4-oxadiazol-3-yl)-2-methylphenyl]oxy}acetate (47 mg). MS (ES): C₂₁H₂₂ClN₃O₅ requires 431; found 432.2 (M+H⁺).

### DESCRIPTION FOR D53

### 3-(3-bromo-2-ethylphenyl)-5-{3-chloro-4-[(1-methylethyl)oxy]phenyl}-1,2,4-oxadiazole (D53)

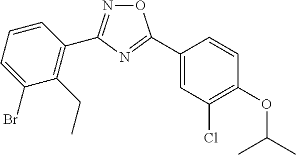

3-chloro-4-[(1-methylethyl)oxy]benzoic acid (105 mg), EDCI (190 mg), HOBT (150 mg) were dissolved in THF (3 mL). After the mixture was stirred at RT for 1 h, a solution of 3-bromo-2-ethyl-N-hydroxybenzenecarboximidamide (D22) (140 mg) in THF (3 ml) was added slowly. The obtained mixture was stirred for further 2 h at room temperature. LCMS show only one intermediate was found. After addition of TBAF (742 mg), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution (twice) and water (twice). After removal of the solvent, the residue was purified by column chromatography to afford 3-(3-bromo-2-ethylphenyl)-5-{3-chloro-4-[(1-methylethyl)oxy]phenyl}-1,2,4-oxadiazole (D53) (345 mg) as a yellow solid. MS (ES): C₁₉H₁₈BrClN₂O₂ requires 420; found 421.1 (M+H⁺).

### DESCRIPTION FOR D54

### ethyl 4-[3-(5-{3-chloro-4-[(1-methylethyl)oxy]phenyl}-1,2,4-oxadiazol-3-yl)-2-ethylphenyl]butanoate (D54)

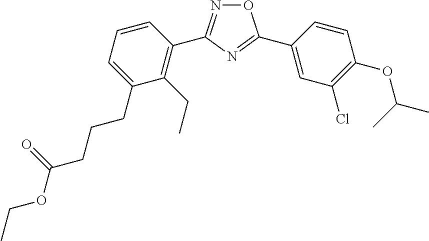

To a suspension of 3-(3-bromo-2-ethylphenyl)-5-{3-chloro-4-[(1-methylethyl)oxy]phenyl}-1,2,4-oxadiazole (D53) (120 mg), tri-t-butylphosphine (17.27 mg) and cesium carbonate (27.8 mg) in tetrahydrofuran (THF) (10 mL) stirred under nitrogen was added Pd₂(dba)₃ (39.1 mg) and a solution of bromo[3-(ethyloxy)-3-oxopropyl]zinc (0.5 M in THF, 1.707 mL) in one portion. The reaction mixture was stirred at RT for 3 h. The solvent was evaporated off and EtOAc (25 mL) was added. The aqueous phase was extracted with EA for 3 times, the combined organic layers were washed with brine, dried over sodium sulphate and evaporated to afford ethyl 4-[3-(5-{3-chloro-4-[(1-methylethyl)oxy]phenyl}-1,2,4-oxadiazol-3-yl)-2-ethylphenyl]butanoate (D54) (173 mg) as a yellow oil, which was used in the next step directly. MS (ES): C₂₅H₂₉ClN₂O₄ requires 456; found 457.2 (M+H⁺).

### DESCRIPTION FOR D55

### ethyl 3-[3-(5-{3-chloro-4-[(1-methylethyl)oxy]phenyl}-1,2,4-oxadiazol-3-yl)-2-ethylphenyl]propanoate (D55)

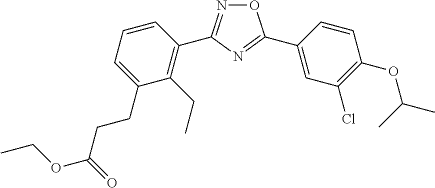

To a suspension of 3-(3-bromo-2-ethylphenyl)-5-{3-chloro-4-[(1-methylethyl)oxy]phenyl}-1,2,4-oxadiazole (D53) (130 mg), tri-t-butylphosphine (19 mg) and cesium carbonate (30 mg) in tetrahydrofuran (THF) (10 mL) stirred under nitrogen was added Pd₂(dba)₃ (42.3 mg) and a solution of bromo[3-(ethyloxy)-3-oxopropyl]zinc (0.5 M in THF, 2.5 mL) in one portion. The reaction mixture was stirred at RT for 3 h. The solvent was evaporated off and EtOAc (25 mL) was added. The aqueous phase was extracted with EA for 3 times, the combined organic layers were washed with brine, dried over sodium sulphate and evaporated to afford ethyl 3-[3-(5-{3-chloro-4-[(1-methylethyl)oxy]phenyl}-1,2,4-oxadiazol-3-yl)-2-ethylphenyl]propanoate (D55) (164 mg) as a yellow oil, which was used in the next step directly. MS (ES): C₂₄H₂₇ClN₂O₄ requires 442; found 443.2 (M+H⁺).

### DESCRIPTION FOR D56

### 3-bromo-5-fluoro-2-(methyloxy)benzonitrile (D56)

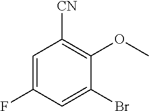

To bromine (3.8 mL) was added iron (0.813 g) in one portion. The reaction mixture was stirred at RT for 5 min, and a solution of 5-fluoro-2-(methyloxy)benzonitrile (2 g) in dichloromethane (DCM) (40 mL) was added dropwise. The reaction mixture was stirred at 60° C. for 24 h. LCMS showed trace of the product. More bromine (3.2 mL) was added. Heating continued at 60° C. for 24 h. Aqueous NaHSO₃ was added to quench the reaction. The aqueous layer was seperated and extracted with EA for 3 times. The combined organic layers were washed with brine, dried over anhydrous Na₂SO₄. The dried solution was purified by column chromatography to afford 3-bromo-5-fluoro-2-(methyloxy)benzonitrile (D56) (330 mg) as an off-white solid. δH (CDCl₃, 400 MHz): 4.04 (3H, s), 7.29 (1H, q), 7.56 (1H, q).

### DESCRIPTION FOR D57

### 3-bromo-5-fluoro-N-hydroxy-2-(methyloxy)benzenecarboximidamide (D57)

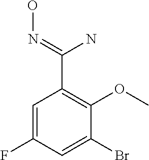

To a solution of 3-bromo-5-fluoro-2-(methyloxy)benzonitrile (D56) (495 mg) in ethanol (8 mL) was added hydroxylamine hydrochloride (301 mg) and sodium bicarbonate (362 mg). The reaction mixture was stirred at 90° C. overnight. After cooling, the reaction, the mixture was filtered through the celite, and washed by ethanol. The filtrate was concentrated in vacuo to afford 3-bromo-5-fluoro-N-hydroxy-2-(methyloxy)benzenecarboximidamide (D57) (573 mg) as an off-white solid. MS (ES): C₈H₈BrFN₂O₂ requires 262; found 263.0 (M+H⁺).

### DESCRIPTION FOR D58

### 5-{3-[3-bromo-5-fluoro-2-(methyloxy)phenyl]-1,2,4-oxadiazol-5-yl}-2-[(1-methylethyl)oxy]benzonitrile (D58)

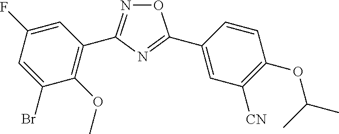

3-cyano-4-[(1-methylethyl)oxy]benzoic acid (202 mg), EDCI (377 mg), HOBT (301 mg) were dissolved in THF (6 mL). After the mixture was stirred at RT for 1 h, a solution of 3-bromo-5-fluoro-N-hydroxy-2-(methyloxy)benzenecarboximidamide (D57) (280 mg) in THF (3 ml) was added slowly. The obtained mixture was stirred for further 2 h at room temperature. LCMS show the intermediate was formed. After addition of TBAF (1.17 g), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution (twice) and water (twice). After removal of the solvent, the residue was purified by column chromatography to afford 5-{3-[3-bromo-5-fluoro-2-(methyloxy)phenyl]-1,2,4-oxadiazol-5-yl}-2-[(1-methylethyl)oxy]benzonitrile (D58) (345 mg) as a yellow solid. MS (ES): C₁₉H₁₅BrFN₃O₃ requires 431; found 432.1 (M+H⁺).

### DESCRIPTION FOR D59

### ethyl 3-[3-(5-{3-cyano-4-[(1-methylethyl)oxy]phenyl}-1,2,4-oxadiazol-3-yl)-5-fluoro-2-(methyloxy)phenyl]propanoate (D59)

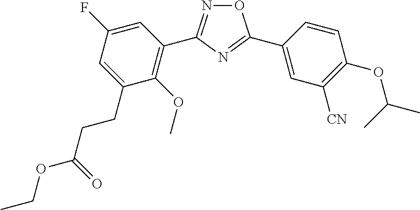

To a suspension of 5-{3-[3-bromo-5-fluoro-2-(methyloxy)phenyl]-1,2,4-oxadiazol-5-yl}-2-[(1-methylethyl)oxy]benzonitrile (D58) (152 mg), tri-t-butylphosphine (21.34 mg) and cesium carbonate (34.4 mg) in tetrahydrofuran (THF) (12 mL) stirred under nitrogen was added Pd₂(dba)₃ (48.3 mg) and a solution of bromo[3-(ethyloxy)-3-oxopropyl]zinc (0.5 M in THF, 2.11 mL) in one portion. The reaction mixture was stirred at RT for 3 h. The solvent was evaporated off and EtOAc (25 mL) was added. The aqueous phase was extracted with EA for 3 times, the combined organic layers were washed with brine, dried over sodium sulphate and evaporated to afford ethyl 3-[3-(5-{3-cyano-4-[(1-methylethyl)oxy]phenyl}-1,2,4-oxadiazol-3-yl)-5-fluoro-2-(methyloxy)phenyl]propanoate (D59) (128 mg) as a yellow oil, which was used in the next step directly. MS (ES): C₂₄H₂₄FN₃O₅ requires 453; found 454.2 (M+H⁺).

### DESCRIPTION FOR D60

### ethyl 4-[3-(5-{3-cyano-4-[(1-methylethyl)oxy]phenyl}-1,2,4-oxadiazol-3-yl)-5-fluoro-2-(methyloxy)phenyl]butanoate (D60)

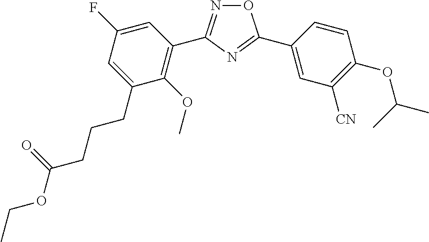

To a suspension of 5-{3-[3-bromo-5-fluoro-2-(methyloxy)phenyl]-1,2,4-oxadiazol-5-yl}-2-[(1-methylethyl)oxy]benzonitrile (D58) (156 mg), tri-t-butylphosphine (21.91 mg) and cesium carbonate (35.3 mg) in tetrahydrofuran (THF) (12 mL) stirred under nitrogen was added Pd₂(dba)₃ (49.6 mg) and a solution of bromo[4-(ethyloxy)-4-oxobutyl]zinc (0.5 M in THF, 2.16 mL) in one portion. The reaction mixture was stirred at RT for 4 h. The solvent was evaporated off and EtOAc (25 mL) was added. The aqueous phase was extracted with EA for 3 times, the combined organic layers were washed with brine, dried over sodium sulphate and evaporated, the residue was purified by column chromatography to afford ethyl 4-[3-(5-{3-cyano-4-[(1-methylethyl)oxy]phenyl}-1,2,4-oxadiazol-3-yl)-5-fluoro-2-(methyloxy)phenyl]butanoate (D60) (130 mg) as a yellow oil. MS (ES): C₂₅H₂₆FN₃O₅ requires 467; found 468.2 (M+H⁺).

### DESCRIPTION FOR D61

### 5-{3-[3-bromo-5-fluoro-2-(methyloxy)phenyl]-1,2,4-oxadiazol-5-yl}-3-chloro-2-[(1-methylethyl)oxy]pyridine (D61)

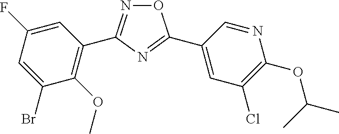

3-chloro-5-[(1-methylethyl)oxy]-2-pyridinecarboxylic acid (205 mg), EDCI (372 mg), HOBT (299 mg) were dissolved in THF (8 mL). After the mixture was stirred at RT for 1 h, a solution of 3-bromo-5-fluoro-N-hydroxy-2-(methyloxy)benzenecarboximidamide (D57) (257 mg) in THF (3 ml) was added slowly. The obtained mixture was stirred for further 2 h at room temperature. LCMS show the intermediate was formed. After addition of TBAF (1.2 g), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution (twice) and water (twice). After removal of the solvent, the residue was purified by column chromatography to afford 5-{3-[3-bromo-5-fluoro-2-(methyloxy)phenyl]-1,2,4-oxadiazol-5-yl}-3-chloro-2-[(1-methylethyl)oxy]pyridine (D61) (316 mg) as a yellow solid. MS (ES): C₁₇H₁₄BrClFN₃O₃ requires 441; found 442.0 (M+H⁺).

### DESCRIPTION FOR D62

### ethyl 4-[3-(5-{5-chloro-6-[(1-methylethyl)oxy]-3-pyridinyl}-1,2,4-oxadiazol-3-yl)-5-fluoro-2-(methyloxy)phenyl]butanoate (D62)

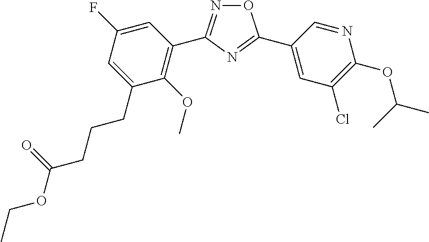

To a suspension of 5-{3-[3-bromo-5-fluoro-2-(methyloxy)phenyl]-1,2,4-oxadiazol-5-yl}-3-chloro-2-[(1-methylethyl)oxy]pyridine (D61) (137 mg), tri-t-butylphosphine (18.78 mg) and cesium carbonate (30.3 mg) in tetrahydrofuran (THF) (8 mL) stirred under nitrogen was added Pd₂(dba)₃ (42.6 mg) and a solution of bromo[4-(ethyloxy)-4-oxobutyl]zinc (0.5 M in THF, 2.2 mL) in one portion. The reaction mixture was stirred at RT overnight. The solvent was evaporated off and EtOAc (25 mL) was added. The aqueous phase was extracted with EA for 3 times, the combined organic layers were washed with brine, dried over sodium sulphate and evaporated, the residue was purified by column chromatography to afford ethyl 4-[3-(5-{5-chloro-6-[(1-methylethyl)oxy]-3-pyridinyl}-1,2,4-oxadiazol-3-yl)-5-fluoro-2-(methyloxy)phenyl]butanoate (D62) (124 mg) as a yellow oil. MS (ES): C₂₃H₂₅ClFN₃O₅ requires 477; found 478.2 (M+H⁺).

### DESCRIPTION FOR D63

### ethyl 3-[3-(5-{5-chloro-6-[(1-methylethyl)oxy]-3-pyridinyl}-1,2,4-oxadiazol-3-yl)-5-fluoro-2-(methyloxy)phenyl]propanoate (D63)

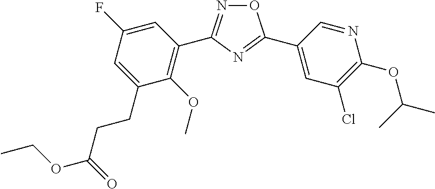

To a suspension of 5-{3-[3-bromo-5-fluoro-2-(methyloxy)phenyl]-1,2,4-oxadiazol-5-yl}-3-chloro-2-[(1-methylethyl)oxy]pyridine (D61) (142 mg), tri-t-butylphosphine (19.47 mg) and cesium carbonate (31.4 mg) in tetrahydrofuran (THF) (12 mL) stirred under nitrogen was added Pd₂(dba)₃ (44.1 mg) and a solution of bromo[3-(ethyloxy)-3-oxopropyl]zinc (0.5 M in THF, 1.93 mL) in one portion. The reaction mixture was stirred at RT overnight. The solvent was evaporated off and EtOAc (25 mL) was added. The aqueous phase was extracted with EA for 3 times, the combined organic layers were washed with brine, dried over sodium sulphate and evaporated, the residue was purified by column chromatography to afford ethyl 3-[3-(5-{5-chloro-6-[(1-methylethyl)oxy]-3-pyridinyl}-1,2,4-oxadiazol-3-yl)-5-fluoro-2-(methyloxy)phenyl]propanoate (D63) (144 mg) as a yellow oil, which was used in the next step directly. MS (ES): C₂₂H₂₃ClFN₃O₅ requires 463; found 464.2 (M+H⁺).

### DESCRIPTION FOR D64

### 3-[3-bromo-5-fluoro-2-(methyloxy)phenyl]-5-{3-chloro-4-[(1-methylethyl)oxy]phenyl}-1,2,4-oxadiazole (D64)

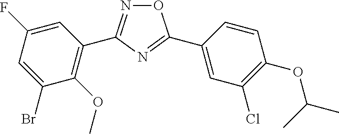

3-chloro-4-[(1-methylethyl)oxy]benzoic acid (200 mg), EDCI (357 mg), HOST (285 mg) were dissolved in THF (4 mL). After the mixture was stirred at RT for 1 h, a solution of 3-bromo-5-fluoro-N-hydroxy-2-(methyloxy)benzenecarboximidamide (D57) (280 mg) in THF (3 ml) was added slowly. The obtained mixture was stirred for further 1 h at room temperature. LCMS show the intermediate was formed. After addition of TBAF (974 mg), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution (twice) and water (twice). After removal of the solvent, the residue was purified by column chromatography to afford 3-[3-bromo-5-fluoro-2-(methyloxy)phenyl]-5-{3-chloro-4-[(1-methylethyl)oxy]phenyl}-1,2,4-oxadiazole (D64) (240 mg) as a white solid. MS (ES): C₁₈H₁₅BrClFN₂O₃ requires 440; found 441.0 (M+H⁺).

### DESCRIPTION FOR D65

### ethyl 3-[3-(5-{3-chloro-4-[(1-methylethyl)oxy]phenyl}-1,2,4-oxadiazol-3-yl)-5-fluoro-2-(methyloxy)phenyl]propanoate (D65)

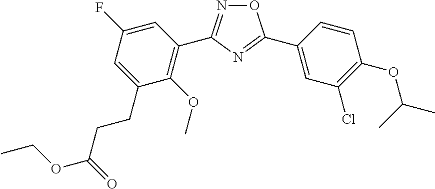

To a suspension of 3-[3-bromo-5-fluoro-2-(methyloxy)phenyl]-5-{3-chloro-4-[(1-methylethyl)oxy]phenyl}-1,2,4-oxadiazole (D64) (115 mg), tri-t-butylphosphine (17 mg) and cesium carbonate (31 mg) in tetrahydrofuran (THF) (10 mL) stirred under nitrogen was added Pd₂(dba)₃ (41 mg) and a solution of bromo[3-(ethyloxy)-3-oxopropyl]zinc (0.5 M in THF, 1.56 mL) in one portion. The reaction mixture was stirred at RT overnight. The solvent was evaporated off and EtOAc (25 mL) was added. The aqueous phase was extracted with EA for 3 times, the combined organic layers were washed with brine, dried over sodium sulphate and evaporated, the residue was purified by column chromatography to afford ethyl 3-[3-(5-{3-chloro-4-[(1-methylethyl)oxy]phenyl}-1,2,4-oxadiazol-3-yl)-5-fluoro-2-(methyloxy)phenyl]propanoate (D65) (109 mg) as a yellow oil. MS (ES): C₂₃H₂₄ClFN₂O₅ requires 462; found 463.2 (M+H⁺).

### DESCRIPTION FOR D66

### ethyl 4-[3-(5-{3-chloro-4-[(1-methylethyl)oxy]phenyl}-1,2,4-oxadiazol-3-yl)-5-fluoro-2-(methyloxy)phenyl]butanoate (D66)

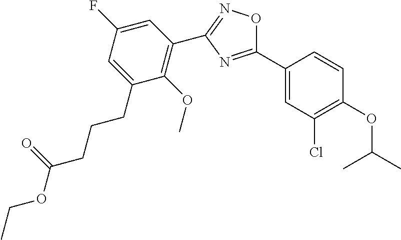

To a suspension of 3-[3-bromo-5-fluoro-2-(methyloxy)phenyl]-5-{3-chloro-4-[(1-methylethyl)oxy]phenyl}-1,2,4-oxadiazole (D64) (72 mg), tri-t-butylphosphine (9.89 mg) and cesium carbonate (18 mg) in tetrahydrofuran (THF) (8 mL) stirred under nitrogen was added Pd₂(dba)₃ (22.39 mg) and a solution of bromo[4-(ethyloxy)-4-oxobutyl]zinc (0.5 M in THF, 0.98 mL) in one portion. The reaction mixture was stirred at RT overnight. The solvent was evaporated off and EtOAc (25 mL) was added. The aqueous phase was extracted with EA for 3 times, the combined organic layers were washed with brine, dried over sodium sulphate and evaporated, the residue was purified by column chromatography to afford ethyl 4-[3-(5-{3-chloro-4-[(1-methylethyl)oxy]phenyl}-1,2,4-oxadiazol-3-yl)-5-fluoro-2-(methyloxy)phenyl]butanoate (D66) (72 mg) as a yellow oil. MS (ES): C₂₄H₂₆ClFN₂O₅ requires 476; found 477.2 (M+H⁺).

### DESCRIPTION FOR D67

### ethyl 4-(3-cyano-2-propylphenyl)butanoate (D67)

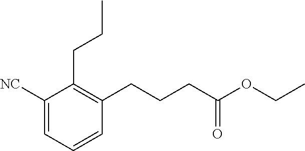

To a suspension of 3-bromo-2-propylbenzonitrile (520 mg), tri-t-butylphosphine (141 mg) and cesium carbonate (243 mg) in tetrahydrofuran (THF) (30 mL) stirred under nitrogen was added Pd₂(dba)₃ (319 mg) and a solution of bromo[4-(ethyloxy)-4-oxobutyl]zinc (0.5 M in THF, 13.92 mL) in one portion. The reaction mixture was stirred at RT overnight. The solvent was diluted with EtOAc, filered through the celite, the filtrate was seperated between aqueous and organic layers. The aqueous phase was extracted with EA for 3 times, the combined organic layers were washed with brine, dried over sodium sulphate and evaporated, the residue was purified by column chromatography to afford ethyl 4-(3-cyano-2-propylphenyl)butanoate (D67) (410 mg) as a yellow oil. MS (ES): C₁₆H₂₁NO₂ requires 259; found 260.2 (M+H⁺).

### DESCRIPTION FOR D68

### ethyl 4-{3-[(Z)-(hydroxyamino)(imino)methyl]-2-propylphenyl}butanoate (D68)

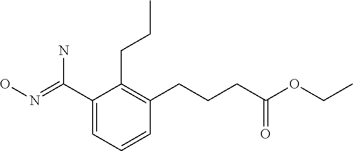

To a solution of ethyl 4-(3-cyano-2-propylphenyl)butanoate (D67) (410 mg) in ethanol (5 mL) was added hydroxylamine hydrochloride (220 mg) and sodium bicarbonate (266 mg). The reaction mixture was stirred at 90° C. overnight. After cooling, the reaction, the mixture was filtered through the celite, and washed by ethanol. The filtrate was concentrated in vacuo to afford ethyl 4-{3-[(Z)-(hydroxyamino)(imino)methyl]-2-propylphenyl}butanoate (D68) (700 mg) as an off-white solid. MS (ES): C₁₆H₂₄N₂O₄ requires 292; found 293.2 (M+H⁺).

### DESCRIPTION FOR D69

### ethyl 4-[3-(5-{3-chloro-4-[(1-methylethyl)oxy]phenyl}-1,2,4-oxadiazol-3-yl)-2-propylphenyl]butanoate (D69)

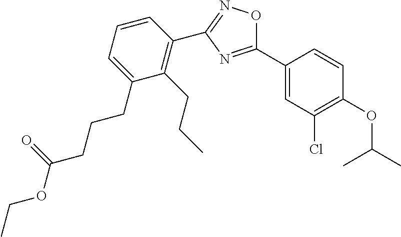

3-chloro-4-[(1-methylethyl)oxy]benzoic acid (140 mg), EDCI (250 mg), HOBT (200 mg) were dissolved in THF (8 mL). After the mixture was stirred at RT for 1 h, a solution of ethyl 4-{3-[(Z)-(hydroxyamino)(imino)methyl]-2-propylphenyl}butanoate (D68) (286 mg) in THF (3 ml) was added slowly. The obtained mixture was stirred for further 1 h at room temperature. LCMS show the intermediate was formed. After addition of TBAF (682 mg), the reaction mixture was sealed and heated under microwave at 120° C. for 1.5 h. The reaction was cooled down to room temperature, the solvent was evaporated and water was added. The aqueous layer was extracted with EtOAc for 3 times, the combined organic layers were washed with brine and dried over Na₂SO₄. After removal of the solvent, the residue was purified by column chromatography to afford ethyl 4-[3-(5-{3-chloro-4-[(1-methylethyl)oxy]phenyl}-1,2,4-oxadiazol-3-yl)-2-propylphenyl]butanoate (D69) (94 mg) as a white solid. MS (ES): C₂₆H₃₁ClN₂O₄ requires 470; found 471.3 (M+H⁺).

### DESCRIPTION FOR D70

### ethyl 4-[3-(5-{3-cyano-4-[(1-methylethyl)oxy]phenyl}-1,2,4-oxadiazol-3-yl)-2-propylphenyl]butanoate (D70)

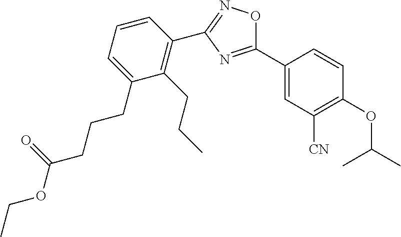

3-cyano-4-[(1-methylethyl)oxy]benzoic acid (139 mg), EDCI (260 mg), HOBT (207 mg) were dissolved in THF (8 mL). After the mixture was stirred at RT for 1 h, a solution of ethyl 4-{3-[(Z)-(hydroxyamino)(imino)methyl]-2-propylphenyl}butanoate (D68) (300 mg) in THF (3 ml) was added slowly. The obtained mixture was stirred for further 1 h at room temperature. LCMS show the intermediate was formed. After addition of TBAF (708 mg), the reaction mixture was sealed and heated under microwave at 120° C. for 2 h. The reaction was cooled down to room temperature, the solvent was evaporated and water was added. The aqueous layer was extracted with EtOAc for 3 times, the combined organic layers were washed with brine and dried over Na₂SO₄. After removal of the solvent, the residue was purified by column chromatography to afford ethyl 4-[3-(5-{3-cyano-4-[(1-methylethyl)oxy]phenyl}-1,2,4-oxadiazol-3-yl)-2-propylphenyl]butanoate (D70) (136 mg) as a yellow oil. MS (ES): C₂₇H₃₁N₃O₄ requires 461; found 462.3 (M+H⁺).

### DESCRIPTION FOR D71

### 3-bromophenyl diethylcarbamate (D71)

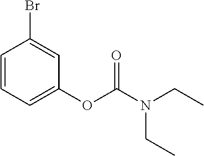

To a mixture of sodium hydride (4.62 g) and THF (80 mL) at 0° C. under nitrogen was added a solution of 3-bromophenol (10 g) in THF (20 mL) dropwise. The mixture was stirred at this temperture for 0.5 h. Then diethylcarbamic chloride (14.65 mL) in THF (20 mL) was added to the mixture dropwise. The mixture was stirred at room temperature for 2 h. The reaction was quenched by EtOH. Then added water. The organic phase was seperated. The aqueous phase was extracted with EA. The combined organic solution was dried over anhydrous sodium sulphate. After concentration, the residue was purified by column chromatography to give 3-bromophenyl diethylcarbamate (D71) (15.4 g) as a oil. MS (ES): C₁₁H₁₄BrNO₂ requires 271; found 272.0 (M+H⁺).

### DESCRIPTION FOR D72

### 3-bromo-2-ethylphenyl diethylcarbamate (D72)

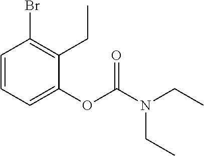

To a solution of 3-bromophenyl diethylcarbamate (D71) (7 g) in tetrahydrofuran (THF) at −78° C. was added sec-butyllithium (35.6 mL) and TMEDA (6.99 mL). The resulting mixture was stirred at this temperature for 1.5 h. After that, iodoethane (6.24 mL) was added to the mixture through syringe. The reaction mixture was warmed to room temperature and stirred at this temperature overnight. The reaction was quenched with water. The organic phase was seperated. The aqueous phase was extracted with EA. The combined organic solution was dried over anhydrous sodium sulphate. After filtration and concentration, the residue was purified by column chromatography to give 3-bromo-2-ethylphenyl diethylcarbamate (D72) (4.4 g). MS (ES): C₁₃H₁₈BrNO₂ requires 299; found 300.1 (M+H⁺).

### DESCRIPTION FOR D73

### 3-bromo-2-ethylphenol (D73)

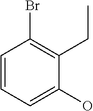

sodium hydroxide (5.86 g) was added into a solution of 3-bromo-2-ethylphenyl diethylcarbamate (D72) (4.4 g) in ethanol (15 mL). The reaction vessel was sealed and heated under microwave at 120° C. for 90 min. After cooling the reaction, the mixture was quenched with water. Then pH value was adjusted to about 5 using 2N HCl solution. EtOH was removed. The residue was extracted with EtOAc. The combined organic solution was dried over anhydrous sodium sulphate. After filtration and concentration, the residue was purified by column chromatography to give 3-bromo-2-ethylphenol (D73) (1.37 g). δH (CDCl₃, 400 MHz): 1.16 (3H, t), 2.82 (2H, m), 5.17 (1H, s), 6.70 (1H, dd), 6.90 (1H, t), 7.12 (1H, dd).

### DESCRIPTION FOR D74

### ethyl 4-[(3-bromo-2-ethylphenyl)oxy]butanoate (D74)

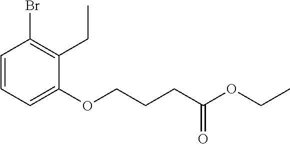

To a solution of 3-bromo-2-ethylphenol (D73) (500 mg) and ethyl 4-bromobutanoate (0.395 mL) in N,N-dimethylformamide (DMF) (10 mL) was added potassium carbonate (447 mg). The reaction mixture was stirred at 65° C. overnight. Water was added to the reaction mixture and then the mixture was extracted with EtOAc. The combined organic solution was dried over anhydrous sodium sulphate. After filtration and concentration, the residue was purified by column chromatography to give ethyl 4-[(3-bromo-2-ethylphenyl)oxy]butanoate (D74) (583 mg). MS (ES): C₁₄H₁₉BrO₃ requires 314; found 315.1 (M+H⁺).

### DESCRIPTION FOR D75

### ethyl 4-[(3-cyano-2-ethylphenyl)oxy]butanoate (D75)

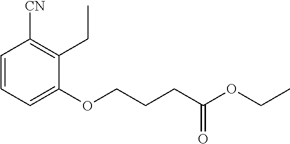

To a solution of ethyl 4-[(3-bromo-2-ethylphenyl)oxy]butanoate (D74) (583 mg) in N-methyl-2-pyrrolidone (NMP) (15 mL) was added copper(I) cyanide (199 mg) and copper(I) iodide (70.5 mg). The mixture was stirred at 180° C. for 6 h. After cooling the reaction, water was added to quench the reaction. The mixture was extracted with EtOAc. The combined organic solution was dried over anhydrous Na₂SO₄. After filtration and concentration, the residue was purified by column chromatography to give ethyl 4-[(3-cyano-2-ethylphenyl)oxy]butanoate (D75) (341.6 mg). MS (ES): C₁₅H₁₉NO₃ requires 261; found 262.2 (M+H⁺).

### DESCRIPTION FOR D76

### ethyl 4-({2-ethyl-3-[(Z)-(hydroxyamino)(imino)methyl]phenyl}oxy)butanoate (D76)

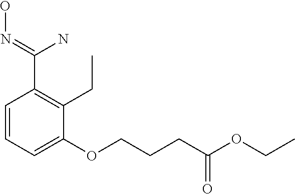

To a solution of ethyl 4-[(3-cyano-2-ethylphenyl)oxy]butanoate (D75) (341 mg) in ethanol (20 mL) was added hydroxylamine hydrochloride (181 mg) and sodium bicarbonate (329 mg). The reaction mixture was stirred at 90° C. overnight. After cooling, the reaction, the mixture was filtered through the celite, and washed by ethanol. The filtrate was concentrated in vacuo to afford ethyl 4-({2-ethyl-3-[(Z)-(hydroxyamino)(imino)methyl]phenyl}oxy)butanoate (D76) (428 mg) as an off-white solid. MS (ES): C₁₅H₂₂N₂O₄ requires 294; found 295.2 (M+H⁺).

### DESCRIPTION FOR D77

### ethyl 4-{[3-(5-{3-cyano-4-[(1-methylethyl)oxy]phenyl}-1,2,4-oxadiazol-3-yl)-2-ethylphenyl]oxy}butanoate (D77)

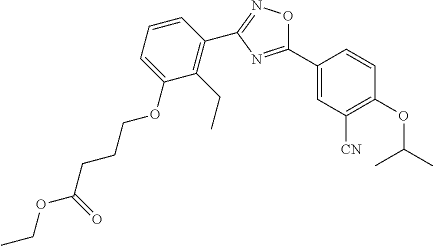

3-cyano-4-[(1-methylethyl)oxy]benzoic acid (100 mg), EDCI (187 mg), HOBT (149 mg) were dissolved in THF (10 mL). After the mixture was stirred at RT for 1 h, a solution of ethyl 4-({2-ethyl-3-[(Z)-(hydroxyamino)(imino)methyl]phenyl}oxy)butanoate (D76) (215 mg) was added. The obtained mixture was stirred for further 2 h at room temperature. LCMS show the intermediate was formed. After addition of TBAF (510 mg), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution and water. After removal of the solvent, the residue was purified by column chromatography to afford ethyl 4-{[3-(5-{3-cyano-4-[(1-methylethyl)oxy]phenyl}-1,2,4-oxadiazol-3-yl)-2-ethylphenyl]oxy}butanoate (D77) (73.5 mg) as a yellow solid. MS (ES): C₂₆H₂₉N₃O₅ requires 463; found 464.3 (M+H⁺).

### DESCRIPTION FOR D78

### ethyl 4-{[3-(5-{3-chloro-4-[(1-methylethyl)oxy]phenyl}-1,2,4-oxadiazol-3-yl)-2-ethylphenyl]oxy}butanoate (D78)

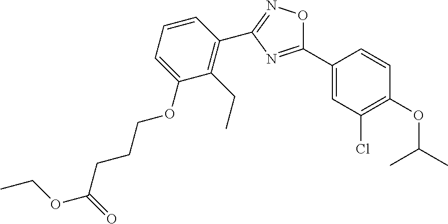

3-chloro-4-[(1-methylethyl)oxy]benzoic acid (100 mg), EDCI (179 mg), HOBT (143 mg) were dissolved in THF (10 mL). After the mixture was stirred at RT for 1 h, a solution of ethyl 4-({2-ethyl-3-[(Z)-(hydroxyamino)(imino)methyl]phenyl}oxy)butanoate (D76) (206 mg) was added. The obtained mixture was stirred for further 2 h at room temperature. LCMS show the intermediate was formed. After addition of TBAF (487 mg), the reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution and water. After removal of the solvent, the residue was purified by column chromatography to afford ethyl 4-{[3-(5-{3-chloro-4-[(1-methylethyl)oxy]phenyl}-1,2,4-oxadiazol-3-yl)-2-ethylphenyl]oxy}butanoate (D78) (84.5 mg). MS (ES): C₂₅H₂₉ClN₂O₅ requires 472; found 473.2 (M+H⁺).

### DESCRIPTION FOR D79

### [2-({[(3-bromo-2-ethylphenyl)oxy]methyl}oxy)ethyl](trimethyl)silane (D79)

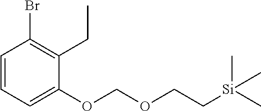

To the solution of 3-bromo-2-ethylphenol (D73) (2.23 g) in dichloromethane (DCM) (50 mL) was added Hunig's base (2.52 mL) followed by SEMCI (2.56 mL) dropwise under ice-water cooling. After addition, the cooling was removed and the reaction solution was warmed to room temperature overnight. The reaction solution was washed with water (2*10 mL), dried over sodium sulphate, concentrated. The crude product was purified by column chromatography to give [2-({[(3-bromo-2-ethylphenyl)oxy]methyl}oxy)ethyl](trimethyl)silane (D79) (3.24 g).

### DESCRIPTION FOR D80

### 2-ethyl-3-hydroxybenzonitrile (D80)

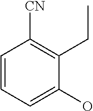

A mixture of [2-({[(3-bromo-2-ethylphenyl)oxy]methyl}oxy)ethyl](trimethyl)silane (D79) (2.74 g), copper(I) cyanide (1.111 g) and copper(I) iodide (0.315 g) in N-methyl-2-pyrrolidone (NMP) (10 mL) was stirred at 180° C. for 6 h. LCMS showed no starting material and the protecting group SEM was removed. The mixture was diluted with EtOAc, the organic phase was washed with water and dried over anhydrous sodium sulphate. After filtration and concentration, the residue was purified by column chromatography to give 2-ethyl-3-hydroxybenzonitrile (D80) (514 mg). MS (ES): C₉H₉NO requires 147; found 148.1 (M+H⁺).

### DESCRIPTION FOR D81

### 2-ethyl-3-[({[2-(trimethylsilyl)ethyl]oxy}methyl)oxy]benzonitrile (D81)

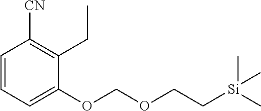

To the solution of 2-ethyl-3-hydroxybenzonitrile (D80) (514 mg) in dichloromethane (DCM) (10 mL) was added Hunig's base (0.732 mL) followed by SEMCI (0.743 mL) dropwise under ice-water cooling. After addition, the cooling was removed and the reaction solution was warmed to room temperature overnight. The reaction solution was washed with water (2*10 mL), dried over sodium sulphate, concentrated. The crude product was purified by column chromatography to give 2-ethyl-3-[({[2-(trimethylsilyl)ethyl]oxy}methyl)oxy]benzonitrile (D81) (950 mg). MS (ES): C₁₅H₂₃NO₂Si requires 277; found 278.2 (M+H⁺).

### DESCRIPTION FOR D82

### 2-ethyl-N-hydroxy-3-[({[2-(trimethylsilyl)ethyl]oxy}methyl)oxy]benzenecarboximidamide (D82)

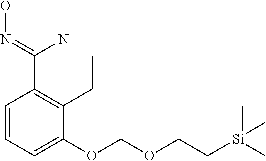

A mixture of 2-ethyl-3-[({[2-(trimethylsilyl)ethyl]oxy}methyl)oxy]benzonitrile (D81) (950 mg), sodium bicarbonate (1.72 g) and hydroxylamine hydrochloride (952 mg) in ethanol (25 mL) was stirred at reflux overnight until the starting material was consumed completely. The mixture was filtered and the filtrate was concentrated. The obtained oil was purified by C18 column to give 2-ethyl-N-hydroxy-3-[({[2-(trimethylsilyl)ethyl]oxy}methyl)oxy]benzenecarboximidamide (D82) (892 mg). MS (ES): C₁₅H₂₆N₂O₃Si requires 310; found 311.2 (M+H⁺).

### DESCRIPTION FOR D83

### 5-(3-{2-ethyl-3-[({[2-(trimethylsilyl)ethyl]oxy}methyl)oxy]phenyl}-1,2,4-oxadiazol-5-yl)-2-[(1-methylethyl)oxy]benzonitrile (D83)

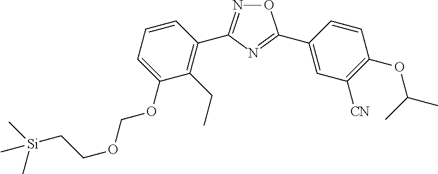

3-cyano-4-[(1-methylethyl)oxy]benzoic acid (350 mg), EDCI (654 mg), HOBT (522 mg) were dissolved in THF (10 mL). After the mixture was stirred at RT for 1 h, a solution of 2-ethyl-N-hydroxy-3-[({[2-(trimethylsilyl)ethyl]oxy}methyl)oxy]benzenecarboximidamide (D82) (794 mg) was added. The reaction mixture was sealed and heated under microwave at 120° C. for 2 h. The reaction was cooled down to room temperature, the solvent was evaporated and EtOAc (100 mL) was added. The organic phase was washed with saturated aqueous sodium dicarbonate solution and water. After removal of the solvent, the residue was purified by column chromatography to afford 5-(3-{2-ethyl-3-[({[2-(trimethylsilyl)ethyl]oxy}methyl)oxy]phenyl}-1,2,4-oxadiazol-5-yl)-2-[(1-methylethyl)oxy]benzonitrile (D83) (364 mg). MS (ES): C₂₆H₃₃N₃O₄Si requires 479; found 480.3 (M+H⁺).

### DESCRIPTION FOR D84

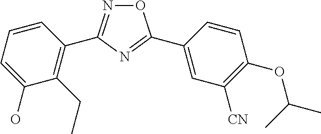

To the solution of 5-(3-{2-ethyl-3-[({[2-(trimethylsilyl)ethyl]oxy}methyl)oxy]phenyl}-1,2,4-oxadiazol-5-yl)-2-[(1-methylethyl)oxy]benzonitrile (D83) (364 mg) in HMPA (5 mL) was added TBAF (992 mg) at room temperature. The resulting solution was stirred at 60° C. overnight. After cooling the reaction, the solution was diluted with ethyl acetate (50 mL), washed with water (3*20 mL), dried over sodium sulphate and concentrated. The residue was purified by column chromatography to give 5-[3-(2-ethyl-3-hydroxyphenyl)-1,2,4-oxadiazol-5-yl]-2-[(1-methylethyl)oxy]benzonitrile (D84) (224.5 mg). MS (ES): C₂₀H₁₉N₃O₃ requires 349; found 350.2 (M+H⁺).

### DESCRIPTION FOR D85

### ethyl {[3-(5-{3-cyano-4-[(1-methylethyl)oxy]phenyl}-1,2,4-oxadiazol-3-yl)-2-ethylphenyl]oxy}acetate (D85)

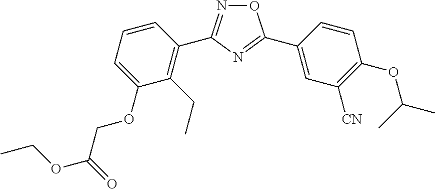

To a solution of 5-[3-(2-ethyl-3-hydroxyphenyl)-1,2,4-oxadiazol-5-yl]-2-[(1-methylethyl)oxy]benzonitrile (D84) (30 mg) in N,N-dimethylformamide (DMF) (2 mL) was added ethyl bromoacetate (28.7 mg) and potassium carbonate (23.73 mg). The resulting mixture was heated to 60° C. for overnight. After cooling the reaction, water was added to the mixture, the aqueous layer was extracted with ethyl acetate for 3 times, the organic phases were washed with water, dried over sodium sulphate, concentrated to afford ethyl {[3-(5-{3-cyano-4-[(1-methylethyl)oxy]phenyl}-1,2,4-oxadiazol-3-yl)-2-ethylphenyl]oxy}acetate (D85) (37.4 mg) as a yellow oil, which was used for the next step directly. MS (ES): C₂₄H₂₅N₃O₅ requires 435; found 436.2 (M+H⁺).

### DESCRIPTION FOR D86

### 5-(5-amino-1,3,4-oxadiazol-2-yl)-2-[(1-methylethyl)oxy]benzonitrile (D86)

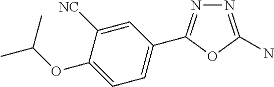

To a suspension of 3-cyano-4-[(1-methylethyl)oxy]benzoic acid (2 g) in phosphorus oxychloride (10 mL) was added hydrazinecarboxamide hydrochloride (1.630 g). The reaction mixture was heated at 90° C. for 3 h. The reaction mixture was cooled to room temperature, the solvent was removed in vacuo. The residue was poured into ice carefully and neutralized with 2 M NaOH to pH=7. The aqueous layer was extracted with EtOAc, the organic layer was dried over sodium sulfate and evaporated in vacuo to afford the crude product 5-(5-amino-1,3,4-oxadiazol-2-yl)-2-[(1-methylethyl)oxy]benzonitrile (D86) (1.93 g). The crude product was used directly for the next step.

### DESCRIPTION FOR D87

### 5-(5-bromo-1,3,4-oxadiazol-2-yl)-2-[(1-methylethyl)oxy]benzonitrile (D87)

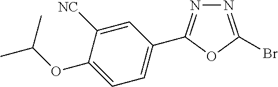

To a mixture of 5-(5-amino-1,3,4-oxadiazol-2-yl)-2-[(1-methylethyl)oxy]benzonitrile (D86) (1.93 g) and copper(II) bromide (3.53 g) in acetonitrile (100 mL), 1,1-dimethylethyl nitrite (1.895 mL) was added in one portion at RT and the reaction mixture was stirred overnight. The reaction mixture was diluted with EtOAc (20 mL) and 1M HCl (10 mL) was added. The organic layer was washed with water and saturated NaHCO₃, then dried over sodium sulphate. The solvent was removed in vacuo. The crude product was purified by column chromatography to give 5-(5-bromo-1,3,4-oxadiazol-2-yl)-2-[(1-methylethyl)oxy]benzonitrile (D87) (1.1 g). MS (ES): C₁₂H₁₀BrN₃O₂ requires 307; found 308.0 (M+H⁺).

### DESCRIPTION FOR D88

### ethyl 4-[3-(5-{3-cyano-4-[(1-methylethyl)oxy]phenyl}-1,3,4-oxadiazol-2-yl)-2-ethylphenyl]butanoate (D88)

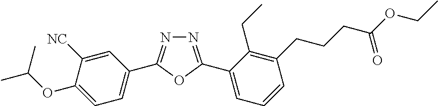

To a suspension of 5-(5-bromo-1,3,4-oxadiazol-2-yl)-2-[(1-methylethyl)oxy]benzonitrile (D87) (64.1 mg), tripotassium phosphate (92 mg) and ethyl 4-[2-ethyl-3-(4,4,5,5-tetramethyl-1,3,2-dioxaborolan-2-yl)phenyl]butanoate (60 mg) in N,N-dimethylformamide (DMF) (4.00 mL) and water (0.8 mL) under nitrogen at room temperature was added Pd(Ph₃P)₄ (20.02 mg) in one charge. The reaction vessel was sealed and heated under microwave at 110° C. for 20 min. After cooling the reaction, the reaction mixture was partitioned between ethyl acetate and water. The organic layers were combined, dried over sodium sulphate and evaporated in vacuo. The residue was purified by column chromatography to give ethyl 4-[3-(5-{3-cyano-4-[(1-methylethyl)oxy]phenyl}-1,3,4-oxadiazol-2-yl)-2-ethylphenyl]butanoate (D88) (40 mg). MS (ES): C₂₆H₂₉N₃O₄ requires 447; found 448.2 (M+H⁺).

### DESCRIPTION FOR D89

### 3-chlorophenyl diethylcarbamate (D89)

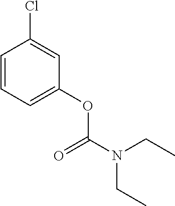

To a solution of 3-chlorophenol (10 g) in tetrahydrofuran (THF) (100 mL) was added NaH (6.22 g) at room temperature. The result suspension was stirred for 2 h. Diethylcarbamic chloride (21.09 g) was added and the reaction mixture was stirred for another 3 h. The reaction was quenched with water (10 mL), washed with brine (20 mL) for 3 times. The organic phase was concentrated and the residue was purified by column chromatography to give 3-chlorophenyl diethylcarbamate (D89) (14.2 g) as a light oil. δH (CDCl₃, 400 MHz): 1.24 (6H, m), 3.41 (4H, m), 7.05 (1H, m), 7.19 (2H, m), 7.27 (1H, m). MS (ES): C₁₁H₁₄ClNO₂ requires 227; found 228.1 (M+H⁺).

### DESCRIPTION FOR D90

### 3-chloro-2-ethylphenyl diethylcarbamate (D90)

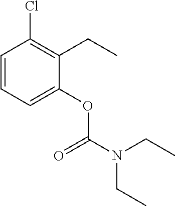

To a solution of TMEDA (16.94 mL) and 3-chlorophenyl diethylcarbamate (D89) (14.2 g) in dry tetrahydrofuran (THF) (25 mL) at −70° C. was added sec-butyllithium (86 mL). The reaction mixture was stirred at this temperature for 2 h. Ethyl iodide (15.12 mL) was added and the reaction mixture was stirred at −70° C. for 1 hour. Then the reaction mixture was warmed to room temperature and stirred overnight. The reaction was quenched with saturated aqueous NH₄Cl (10 mL) and partitioned between brine (10 mL) and ethyl acetate (50 mL). The organic phase was concentrated and the residue was purified by column chromatography to give 3-chloro-2-ethylphenyl diethylcarbamate (D90) (11.7 g) as a light yellow oil. MS (ES): C₁₃H₁₈ClNO₂ requires 255; found 256.1 (M+H⁺).

### DESCRIPTION FOR D91

### 3-chloro-2-ethylphenol (D91)

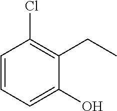

To a solution of 3-chloro-2-ethylphenyl diethylcarbamate (D90) (2.5 g) in ethanol (8 mL) was added NaOH (2.5 g) at room temperature. The reaction vessel was sealed and heated under microwave at 120° C. for 90 min. The mixture was concentrated to remove the solvent and the pH value was adjusted to about 6 with conc. HCl and 2M HCl under ice bath. The mixture was partitioned between ethyl acetate and brine. The organic phase was concentrated to give 3-chloro-2-ethylphenol (D91) (990 mg) as a light brown oil. δH (DMSO-d₆, 400 MHz): 1.09 (3H, t), 2.71 (2H, m), 6.80 (1H, d), 6.85 (1H, d), 7.03 (1H, t), 9.80 (1H, s).

### DESCRIPTION FOR D92

### 3-chloro-2-ethylphenyl trifluoromethanesulfonate (D92)

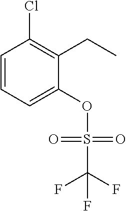

To the mixture of 3-chloro-2-ethylphenol (D91) (12.4 g) and DMAP (13.54 g) in dichloromethane (DCM) (50 mL) was added 1,1,1-trifluoro-N-phenyl-N-[trifluoromethyl)sulfonyl]methanesulfonamide (28.3 g). The reaction mixture was stirred at room temperature overnight. Water (1 mL) was added and the mixture was stirred for 2 min. The resulting mixture was concentrated to remove the solvent. Petroleum ether (50 mL) was added and most of suspenstion emerged. The mixture was filtered, the filtrate was concentrated and the residue was purified by column chromatography to give 3-chloro-2-ethylphenyl trifluoromethanesulfonate (D92) (10.0 g). δH (CDCl₃, 400 MHz): 1.15 (3H, t), 2.80 (2H, m), 7.14 (2H, m), 7.33 (1H, m).

### DESCRIPTION FOR D93

### ethyl 4-(3-chloro-2-ethylphenyl)butanoate (D93)

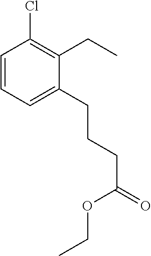

To the mixture of Pd₂(dba)₃ (0.095 g) and 1,1′-bis(diphenylphosphino)ferrocene (0.058 g) in tetrahydrofuran (THF) (8 mL) was added 3-chloro-2-ethylphenyl trifluoromethanesulfonate (D92) (1.5 g) and bromo[4-(ethyloxy)-4-oxobutyl]zinc (18.71 mL) under nitrogen at room temperature. The reaction vessel was sealed and heated under microwave at 120° C. for 45 min. The reaction was quenched with water, the mixture was concentrated in vacuo and the residue was purified by column chromatography to give ethyl 4-(3-chloro-2-ethylphenyl)butanoate (D93) (0.66 g) as a brown oil. MS (ES): C₁₄H₁₉ClO₂ requires 254; found 255.1 (M+H⁺).

### DESCRIPTION FOR D94

### ethyl 4-[2-ethyl-3-(4,4,5,5-tetramethyl-1,3,2-dioxaborolan-2-yl)phenyl]butanoate (D94)

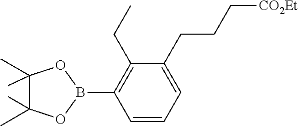

Tricyclohexylphosphine (0.550 g) and Pd₂(dba)₃ (0.144 g) in N,N-dimethylformamide (DMF) (12 mL) were stirred under nitrogen for 30 min. Ethyl 4-(3-chloro-2-ethylphenyl)butanoate (D93) (1 g), 4,4,4′,4′,5,5,5′,5′-octamethyl-2,2′-bi-1,3,2-dioxaborolane (1.794 g) and potassium acetate (0.770 g) were added to the reacion mixture under nitrogen at room temperature. The reaction vessel was sealed and heated under microwave at 180° C. for 90 min. After cooling the reaction, the reaction was filtered, the filtrate was concentrated in vacuo and the residue was purified by column chromatography to give ethyl 4-[2-ethyl-3-(4,4,5,5-tetramethyl-1,3,2-dioxaborolan-2-yl)phenyl]butanoate (D94) (0.617 g) as a brown oil. δH (CDCl₃, 400 MHz): 1.15 (3H, t), 1.28 (3H, t), 1.36 (12H, s), 1.92 (2H, m), 2.38 (2H, t), 2.68 (2H, t), 2.96 (2H, m), 4.15 (2H, m), 7.13 (1H, m), 7.21 (1H, d), 7.63 (1H, d). MS (ES): C₂₀H₃₁BO₄ requires 346; found 347.3 (M+H⁺).

### DESCRIPTION FOR D95

### 5-(5-amino-1,3,4-thiadiazol-2-yl)-2-[(1-methylethyl)oxy]benzonitrile (D95)

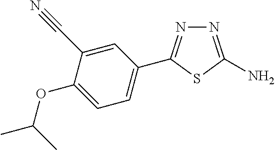

To a mixture of 3-cyano-4-[(1-methylethyl)oxy]benzoic acid (2 g) and hydrazinecarbothioamide (1.332 g) was added POCl₃ (20 mL). The reaction mixture was stirred at 90° C. for 3 h. After cooling the reaction, the mixture was concentrated to remove POCl₃. The residue was partitioned between ethyl acetate (100 mL) and water (100 mL). The organic phase was washed with water (50 mL), 2M sodium hydroxide solution (50 mL) and saturated brine (50 mL), dried over sodium sulphate and evaporated to give the crude product 5-(5-amino-1,3,4-thiadiazol-2-yl)-2-[(1-methylethyl)oxy]benzonitrile (D95) (2 g). MS (ES): C₁₂H₁₂N₄OS requires 260; found 261.1 (M+H⁺).

### DESCRIPTION FOR D96

5-(5-bromo-1,3,4-thiadiazol-2-yl)-2-[(1-methylethyl)oxy]benzonitrile (D96)

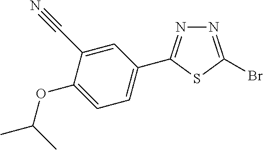

To a suspension of 5-(5-amino-1,3,4-thiadiazol-2-yl)-2-[(1-methylethyl)oxy]benzonitrile (D95) (800 mg), copper(II) bromide (1373 mg) in acetonitrile (10 mL) was added 1,1-dimethylethyl nitrite (0.737 mL). The reaction mixture was stirred at 20° C. for 1.5 h. The reaction was quenched with aqueous HCl (2M), the mixture was partitioned between ethyl acetate (50 mL) and water (25 mL). The organic phase was washed with water (25 mL) and saturated brine (25 mL), dried over sodium sulphate and evaporated in vacuo, the residue was purified by column chromatography to give 5-(5-bromo-1,3,4-thiadiazol-2-yl)-2-[(1-methylethyl)oxy]benzonitrile (D96) (800 mg). MS (ES): C₁₂H₁₀BrN₃OS requires 323; found 324.0 (M+H⁺).

### DESCRIPTION FOR D97

### ethyl 4-[3-(5-{3-cyano-4-[(1-methylethyl)oxy]phenyl}-1,3,4-thiadiazol-2-yl)-2-ethylphenyl]butanoate (D97)

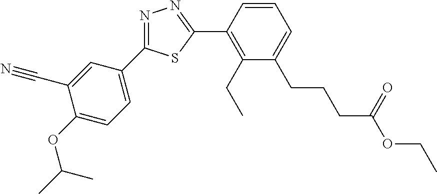

To a solution of 5-(5-bromo-1,3,4-thiadiazol-2-yl)-2-[(1-methylethyl)oxy]benzonitrile (D96) (57.1 mg), ethyl 4-[2-ethyl-3-(4,4,5,5-tetramethyl-1,3,2-dioxaborolan-2-yl)phenyl]butanoate (D94) (61 mg) and tripotassium phosphate (93 mg) in N,N-dimethylformamide (DMF) (6 mL) and water (1.5 mL) stirred under nitrogen at room temperature was added Pd(Ph₃P)₄ (20.36 mg). The reaction vessel was sealed and heated under microwave at 130° C. for 8 min. After cooling the reaction, the reaction was quenched with water. After filtration, the filtrate was partitioned between ethyl acetate and aqueous layer. The aqueous layer was extracted with EA for 3 times. The combined organic layers were washed with brine, dried over anhydrous sodium sulfate and concentrated, the residue was purified by column chromatography to afford ethyl 4-[3-(5-{3-cyano-4-[(1-methylethyl)oxy]phenyl}-1,3,4-thiadiazol-2-yl)-2-ethylphenyl]butanoate (D97) (59 mg) as a yellow oil. MS (ES): C₂₆H₂₉N₃O₃S requires 463; found 464.2 (M+H⁺).

### DESCRIPTION FOR D98

### 2-ethyl-3-(4,4,5,5-tetramethyl-1,3,2-dioxaborolan-2-yl)benzaldehyde (D98)

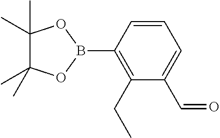

To a solution of 3-bromo-2-ethylbenzaldehyde (2 g) in N,N-dimethylformamide (DMF) (20 mL) was added 4,4,4′,4′,5,5,5′,5′-octamethyl-2,2′-bi-1,3,2-dioxaborolane (3.10 g), potassium acetate (2.76 g) and PdCl₂(dppf)-CH₂Cl₂ adduct (1.150 g). The reaction mixture was heated to 80° C. overnight. The solvent was removed in vacuo and the residue was purified by column chromatography to afford 2-ethyl-3-(4,4,5,5-tetramethyl-1,3,2-dioxaborolan-2-yl)benzaldehyde (D98) (1.27 g). MS (ES): C₁₅H₂₁BO₃ requires 260; found 261.2 (M+H⁺).

### DESCRIPTION FOR D99

### 5-{3-chloro-4-[(1-methylethyl)oxy]phenyl}-1,3,4-thiadiazol-2-amine (D99)

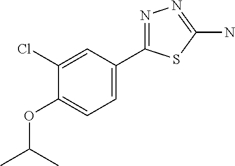

The mixture of 3-chloro-4-[(1-methylethyl)oxy]benzoic acid (5 g), hydrazinecarbothioamide (2.229 g) in phosphoric trichloride (21.43 g) was stirred at 75° C. for 3 h. Concentrated the mixture in vacuum to remove POCl₃, the residue was poured into crush ice. Basified the mixture with aqueous NaOH. Extracted it with EA twice. The EA layer was concentrated in vacuum to give crude product 5-{3-chloro-4-[(1-methylethyl)oxy]phenyl}-1,3,4-thiadiazol-2-amine (D99) (7.5 g), which was used in the next step without further purification. MS (ES): C₁₁H₁₂ClN₃OS requires 269; found 270.1 (M+H⁺).

### DESCRIPTION FOR D100

### 2-bromo-5-{3-chloro-4-[(1-methylethyl)oxy]phenyl}-1,3,4-thiadiazole (D100)

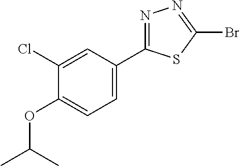

The mixture of 5-{3-chloro-4-[(1-methylethyl)oxy]phenyl}-1,3,4-thiadiazol-2-amine (D99) (6.25 g), copper(II) bromide (10.35 g) in acetonitrile (60 mL) was stirred at room temperature, then 1,1-dimethylethyl nitrite (5.51 mL) was added. The mixture was stirred at room temperature for 3 h. It was treated with EA and aqueous HCl, the EA layer was combined and purified by column chromatography to give 2-bromo-5-{3-chloro-4-[(1-methylethyl)oxy]phenyl}-1,3,4-thiadiazole (D100) (4.76 g). MS (ES): C₁₁H₁₀BrClN₂OS requires 333; found 334.0 (M+H⁺).

### DESCRIPTION FOR D101

### 3-(5-{3-chloro-4-[(1-methylethyl)oxy]phenyl}-1,3,4-thiadiazol-2-yl)-2-ethylbenzaldehyde (D101)

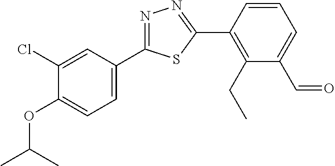

To a suspension of 2-bromo-5-{3-chloro-4-[(1-methylethyl)oxy]phenyl}-1,3,4-thiadiazole (D100) (0.5 g), 2-ethyl-3-(4,4,5,5-tetramethyl-1,3,2-dioxaborolan-2-yl)benzaldehyde (D98) (0.468 g) and tripotassium phosphate (0.795 g) in N,N-dimethylformamide (DMF) (10 mL) and water (1.5 mL) stirred under nitrogen at room temperature was added Pd(Ph₃P)₄ (0.173 g). The reaction vessel was sealed and heated under microwave at 130° C. for 10 min. After cooling the reaction, water was added to the reaction mixture. The mixture was extracted with EA. The combined organic solution was dried over anhydrous sodium sulphate. After filtration and concentration, the residue was purified by column chromatography to give 3-(5-{3-chloro-4-[(1-methylethyl)oxy]phenyl}-1,3,4-thiadiazol-2-yl)-2-ethylbenzaldehyde (D101) (164 mg). MS (ES): C₂₀H₁₉ClN₂O₂S requires 386; found 387.1 (M+H⁺).

### DESCRIPTION FOR D102

### 5-bromo-2-[(1-methylethyl)oxy]benzonitrile (D102)

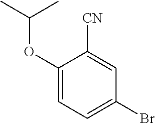

To a solution of 5-bromo-2-hydroxybenzonitrile (25 g) in acetonitrile (150 mL) was added 2-iodopropane (15.14 mL) and potassium carbonate (34.9 g). The reaction mixture was stirred at room temperature for two days. The solvent was removed in vacuo, the residue was dissolved in ethyl acetate (150 mL), washed with water (2*30 mL), the organic phase was dried over sodium sulphate and concentrated to afford 5-bromo-2-[(1-methylethyl)oxy]benzonitrile (D102) (29.8 g) as a white solid without further purification. δH (CDCl₃, 400 MHz): 1.39 (6H, d), 4.61 (1H, m), 6.85 (1H, d), 7.58 (1H, dd), 7.64 (1H, d). MS (ES): C₁₀H₁₀BrNO requires 239; found 240.0 (M+H⁺).

### DESCRIPTION FOR D103

2-[(1-methylethyl)oxy]-5-(4,4,5,5-tetramethyl-1,3,2-dioxaborolan-2yl)benzonitrile (D103)

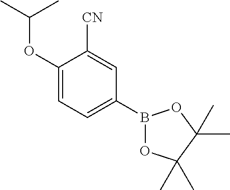

To a suspension of 5-bromo-2-[(1-methylethyl)oxy]benzonitrile (D102) (123 mg), 4,4,4′,4′,5,5,5′,5′-octamethyl-2,2′-bi-1,3,2-dioxaborolane (156 mg) and potassium acetate (101 mg) in N,N-dimethylformamide (DMF) (150 mL) stirred under nitrogen at room temperature was added PdCl₂(dppf)-CH₂Cl₂ adduct (25.1 mg). The reaction vessel was sealed and heated under microwave at 120° C. for 1 h. After cooling the reaction, the reaction mixture was concentrated in vacuo and the residue was purified by column chromatography to give 2-[(1-methylethyl)oxy]-5-(4,4,5,5-tetramethyl-1,3,2-dioxaborolan-2-yl)benzonitrile (D103) (54 mg). MS (ES): C₁₆H₂₂BNO₃ requires 287; found 288.2 (M+H⁺).

### DESCRIPTION FOR D104

### ethyl 4-[3-(3-{3-cyano-4-[(1-methylethyl)oxy]phenyl}-1,2,4-thiadiazol-5-yl)-2-ethylphenyl]butanoate (D104)

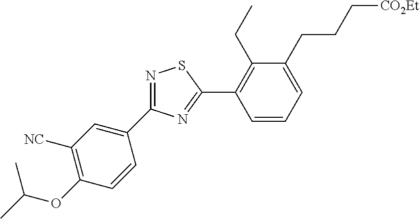

To a suspension of 2-[(1-methylethyl)oxy]-5-(4,4,5,5-tetramethyl-1,3,2-dioxaborolan-2-yl)benzonitrile (D103) (74.9 mg), ethyl 4-[3-(3-bromo-1,2,4-thiadiazol-5-yl)-2-ethylphenyl]butanoate (D35) (100 mg) and tripotassium phosphate (138 mg) in 1,2-dimethoxyethane (DME) (4 mL) and water (1 mL) under nitrogen was added Pd(Ph₃P)₄ (30.1 mg). The reaction vessel was sealed and heated under microwave at 120° C. for 10 min. The reaction mixture was concentrated to give the crude product ethyl 4-[3-(3-{3-cyano-4-[(1-methylethyl)oxy]phenyl}-1,2,4-thiadiazol-5-yl)-2-ethylphenyl]butanoate (D104) (61 mg), which was directly used for the next step without further purification. MS (ES): C₂₆H₂₉N₃O₃S requires 463; found 464.2 (M+H⁺).

### DESCRIPTION FOR D105

### 3-bromo-5-{3-chloro-4-[(1-methylethyl)oxy]phenyl}-1,2,4-thiadiazole (D105)

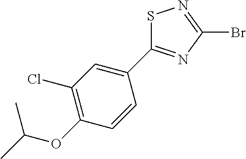

To a solution of 3-bromo-5-chloro-1,2,4-thiadiazole (558 mg), {3-chloro-4-[(1-methylethyl)oxy]phenyl}boronic acid (300 mg) and tripotassium phosphate (148 mg) in N,N-dimethylformamide (DMF) (3 mL) and water (0.600 mL) under nitrogen was added PdCl₂(dppf)-CH₂Cl₂ adduct (3.43 g). The reaction vessel was sealed and heated under microwave at 80° C. for 1 h. After cooling the reaction, the mixture was filtered and the filtrate was diluted with ethyl acetate (50 mL), washed with brine (2*10 mL), dried over sodium sulphate, concentrated and purified by column chromatography to afford 3-bromo-5-{3-chloro-4-[(1-methylethyl)oxy]phenyl}-1,2,4-thiadiazole (D105) (450 mg) as a clear oil. MS (ES): C₁₁H₁₀BrClN₂OS requires 332; found 333.0 (M+H⁺).

### DESCRIPTION FOR D106

### ethyl 4-[3-(5-{3-chloro-4-[(1-methylethyl)oxy]phenyl}-1,2,4-thiadiazol-3-yl)-2-ethylphenyl]butanoate (D106)

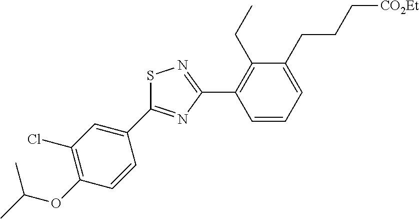

To a mixture of ethyl 4-[2-ethyl-3-(4,4,5,5-tetramethyl-1,3,2-dioxaborolan-2-yl)phenyl]butanoate (D94) (50 mg), 3-bromo-5-{3-chloro-4-[(1-methylethyl)oxy]phenyl}-1,2,4-thiadiazole (D105) (53 mg) and potassium phosphate (61.3 mg) in N,N-dimethylformamide (DMF) (4 mL) and water (1 mL) under nitrogen was added Pd(Ph₃P)₄ (30 mg). The reaction vessel was sealed and heated under microwave at 130° C. for 8 min. The organic layer was concentrated and purified by column chromatography to give ethyl 4-[3-(5-{3-chloro-4-[(1-methylethyl)oxy]phenyl}-1,2,4-thiadiazol-3-yl)-2-ethylphenyl]butanoate (D106) (51 mg,) as a brown solid. MS (ES): C₂₅H₂₉ClN₂O₃S requires 472; found 473.2 (M+H⁺).

### DESCRIPTION FOR D107

### 5-(3-bromo-1,2,4-thiadiazol-5-yl)-2-[(1-methylethyl)oxy]benzonitrile (D107)

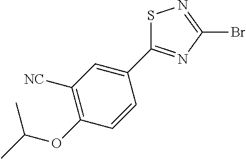

To a solution of 2-[(1-methylethyl)oxy]-5-(4,4,5,5-tetramethyl-1,3,2-dioxaborolan-2-yl)benzonitrile (D103) (280 mg), 3-bromo-5-chloro-1,2,4-thiadiazole (194 mg) and tripotassium phosphate (517 mg) in 1,2-dimethoxyethane (DME) (4 mL) and water (1 mL) under nitrogen was added Pd(Ph₃P)₄ (113 mg). The reaction vessel was sealed and heated under microwave at 120° C. for 10 min. Water was added, the reaction mixture was filtered through the celite. The aqueous layer was extracted with EA for 3 times. the combined organic layers were washed with brine, dried over anhydrous sodium sulfate. The dried solution was concentrated and purified by column chromatography to give 5-(3-bromo-1,2,4-thiadiazol-5-yl)-2-[(1-methylethyl)oxy]benzonitrile (D107) (152 mg) as a white solid. MS (ES): C₁₂H₁₀BrN₃OS requires 323; found 324.0 (M+H⁺).

### DESCRIPTION FOR D108

### ethyl 4-[3-(5-{3-cyano-4-[(1-methylethyl)oxy]phenyl}-1,2,4-thiadiazol-3-yl)-2-ethylphenyl]butanoate (D108)

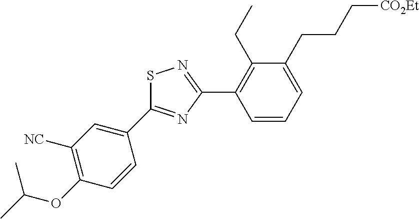

To a suspension of 5-(3-bromo-1,2,4-thiadiazol-5-yl)-2-[(1-methylethyl)oxy]benzonitrile (D107) (69 mg), ethyl 4-[2-ethyl-3-(4,4,5,5-tetramethyl-1,3,2-dioxaborolan-2-yl)phenyl]butanoate (D94) (73 mg) and tripotassium phosphate (112 mg) in N,N-dimethylformamide (DMF) (4 mL) and water (1 mL) under nitrogen was added Pd(Ph₃P)₄ (24.36 mg). The reaction vessel was sealed and heated under microwave at 120° C. for 10 min. Water was added, the reaction mixture was filtered through the celite. The aqueous layer was extracted with EA for 3 times, the combined organic layers were washed with brine, dried over anhydrous sodium sulfate. The dried solution was concentrated to afford the crude product ethyl 4-[3-(5-{3-cyano-4-[(1-methylethyl)oxy]phenyl}-1,2,4-thiadiazol-3-yl)-2-ethylphenyl]butanoate (D108) (98 mg). MS (ES): C₂₆H₂₉N₃O₃S requires 463; found 464.3 (M+H⁺).

### DESCRIPTION FOR D109

### 5-[3-(2-ethyl-3-formylphenyl)-1,2,4-thiadiazol-5-yl]-2-[(1-methylethyl)oxy]benzonitrile (D109)

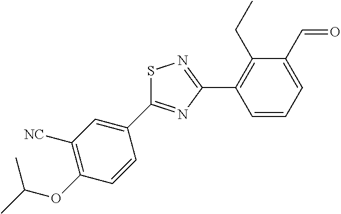

To a solution of 5-(3-bromo-1,2,4-thiadiazol-5-yl)-2-[(1-methylethyl)oxy]benzonitrile (D107) (550 mg), 2-ethyl-3-(4,4,5,5-tetramethyl-1,3,2-dioxaborolan-2-yl)benzaldehyde (463 mg) and tripotassium phosphate (1080 mg) in N,N-dimethylformamide (DMF) (4 mL) and water (1.000 mL) stirred under nitrogen was added Pd(Ph₃P)₄ (196 mg). The reaction was sealed and heated under microwave at 120° C. for 15 min. After cooling, the reaction mixture was diluted with ethyl acetate (20 mL), washed with water (2*8 mL), dried over sodium sulphate, concentrated and purified by column chromatography to afford 5-[3-(2-ethyl-3-formylphenyl)-1,2,4-thiadiazol-5-yl]-2-[(1-methylethyl)oxy]benzonitrile (D109) (420 mg) as a solid. MS (ES): C₂₁H₁₉N₃O₂S requires 377; found 378.0 (M+H⁺).

### DESCRIPTION FOR D110

### 5-{3-chloro-4-[(1-methylethyl)oxy]phenyl}-3-{2-ethyl-3-[(E)-2-(methyloxy)ethenyl]phenyl}-1,2,4-thiadiazole (D110)

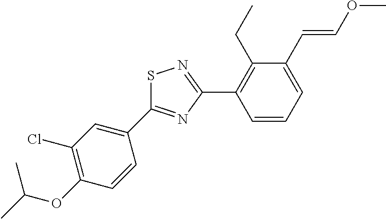

To a solution of 3-bromo-5-{3-chloro-4-[(1-methylethyl)oxy]phenyl}-1,2,4-thiadiazole (D105) (388 mg), 2-{2-ethyl-3-[(E)-2-(methyloxy)ethenyl]phenyl}-4,4,5,5-tetramethyl-1,3,2-dioxaborolane (335 mg) and tripotassium phosphate (741 mg) in N,N-dimethylformamide (DMF) (10 mL) and water (1.000 mL) stirred under nitrogen was added Pd(Ph₃P)₄ (134 mg). The mixture was sealed and heated under microwave at 120° C. for 10 min. After cooling the reaction, the mixture was concentrated and purified by column chromatography to give 5-{3-chloro-4-[(1-methylethyl)oxy]phenyl}-3-{2-ethyl-3-[(E)-2-(methyloxy)ethenyl]phenyl}-1,2,4-thiadiazole (D110) (313 mg) as a colorless oil. MS (ES): C₂₂H₂₃ClN₂O₂S requires 414; found 415.1 (M+H⁺).

### DESCRIPTION FOR D111

### [3-(5-{3-chloro-4-[(1-methylethyl)oxy]phenyl}-1,2,4-thiadiazol-3-yl)-2-ethylphenyl]acetaldehyde (D111)

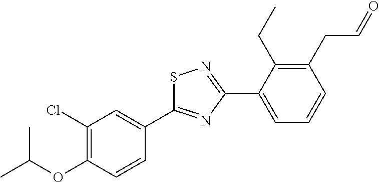

To a solution of 5-{3-chloro-4-[(1-methylethyl)oxy]phenyl}-3-{2-ethyl-3-[(E)-2-(methyloxy)ethenyl]phenyl}-1,2,4-thiadiazole (D110) (313 mg) in tetrahydrofuran (THF) (20 mL) stirred under nitrogen at room temperature was added 14 drops 2M hydrochloric acid. The reaction mixture was stirred at 70° C. for 4 h. After cooling the reaction, the solution was condensed under reduced pressure to give [3-(5-{3-chloro-4-[(1-methylethyl)oxy]phenyl}-1,2,4-thiadiazol-3-yl)-2-ethylphenyl]acetaldehyde (D111) (300 mg), which was used directly in the next step without further purification. MS (ES): C₂₁H₂₁ClN₂O₂S requires 400; found 401.1 (M+H⁺).

### DESCRIPTION FOR D112

### 1-bromo-5-fluoro-2-(methyloxy)-3-nitrobenzene (D112)

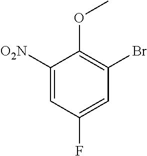

To a stirred solution of 2-bromo-4-fluoro-1-(methyloxy)benzene (8 g) in sulfuric acid (31.2 ml) at −10° C. was added nitric acid (2.79 ml) dropwise. After stirring for 30 min, the mixture was poured into ice, extracted with EtOAc (4*50 mL). The combined organic phases were washed with brine, dried over Na₂SO₄ and concentrated, the residue was purified by column chromatography to afford 1-bromo-5-fluoro-2-(methyloxy)-3-nitrobenzene (D112) (4.09 g). δH (CDCl₃, 400 MHz): 4.00 (3H, t), 7.55 (2H, m).

### DESCRIPTION FOR D113

### ethyl 4-[5-fluoro-2-(methyloxy)-3-nitrophenyl]butanoate (D113)

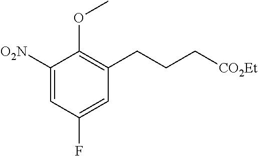

To a suspension of 1-bromo-5-fluoro-2-(methyloxy)-3-nitrobenzene (D112) (4.09 g), cesium carbonate (2.132 g) and tri-t-butylphosphine (1.898 g) in tetrahydrofuran (THF) (30 mL) under nitrogen was added bromo[4-(ethyloxy)-4-oxobutyl]zinc (65.4 mL) followed by Pd₂(dba)₃ (1.498 g). The reaction mixture was stirred at room temperature overnight. Saturated aqueous ammonium chloride solution was added to quench the reaction. The aqueous phase was extracted with ethyl acetate. The combined organic phases were dried over anhydrous sodium sulphate and concentrated, the residue was purified by column chromatography to give ethyl 4-[5-fluoro-2-(methyloxy)-3-nitrophenyl]butanoate (D113) (3.64 g). MS (ES): C₁₃H₁₆FNO₅ requires 285; found 286.1 (M+H⁺).

### DESCRIPTION FOR D114

### ethyl 4-[3-amino-5-fluoro-2-(methyloxy)phenyl]butanoate (D114)

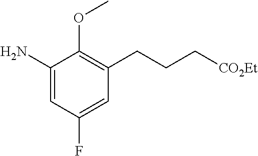

A mixture of ethyl 4-[5-fluoro-2-(methyloxy)-3-nitrophenyl]butanoate (D113) (4.19 g), iron (8.20 g) and saturated aqueous ammonium chloride solution (20 mL) was stirred at 90° C. overnight. After cooling the reaction, the reaction mixture was filtered and the filtrate was evaporated to remove organic solvent, the residue was extracted with ethyl acetate. The combined organic phases were dried over anhydrous sodium sulphate and concentrated, the residue was purified by column chromatography to give ethyl 4-[3-amino-5-fluoro-2-(methyloxy)phenyl]butanoate (D114) (2.27 g). MS (ES): C₁₃H₁₈FNO₃ requires 255; found 256.2 (M+H⁴).

### DESCRIPTION FOR D115

### ethyl 4-[3-bromo-5-fluoro-2-(methyloxy)phenyl]butanoate (D115)

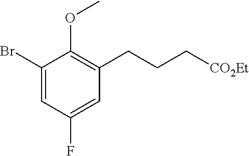

To a solution of ethyl 4-[3-amino-5-fluoro-2-(methyloxy)phenyl]butanoate (D114) (1.7 g) in acetonitrile (50 mL) was added HBr (7.23 mL) at 0° C. Then a solution of sodium nitrite (0.919 g) in water (5 mL) was added to the reaction mixture. After stirring for 10 min, copper(II) bromide (2.97 g) and copper(I) bromide (0.191 g) were added, The reaction mixture was stirred at 50° C. for 1 h. After cooling the reaction, aqueous NH₄Cl solution was added, the resulting suspension was extracted with ethyl acetate. The combined organic phases were dried over anhydrous sodium sulphate and concentrated. The residue was purified by column chromatography to give ethyl 4-[3-bromo-5-fluoro-2-(methyloxy)phenyl]butanoate (D115) (1.84 g). MS (ES): C₁₃H₁₆BrFO₃ requires 318; found 319.1 (M+H⁺).

### DESCRIPTION FOR D116

### ethyl 4-[5-fluoro-2-(methyloxy)-3-(4,4,5,5-tetramethyl-1,3,2-dioxaborolan-2-yl)phenyl]butanoate (D116)

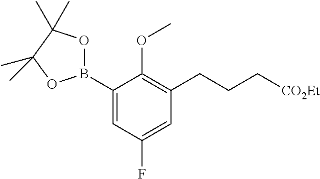

To a solution of ethyl 4-[3-bromo-5-fluoro-2-(methyloxy)phenyl]butanoate (D115) (234 mg), 4,4,4′,4′,5,5,5′,5′-octamethyl-2,2′-bi-1,3,2-dioxaborolane (335 mg), tricyclohexylphosphine (103 mg) and potassium acetate (144 mg) in N,N-dimethylformamide (DMF) (2 mL) was added Pd₂dba₃ (26.9 mg) under nitrogen. The reaction vessel was sealed and heated under microwave at 150° C. for 45 min. After cooling the reaction, the mixture was diluted with brine, extracted with ethyl acetate, the combined organic phases were dried over anhydrous sodium sulphate and concentrated, the residue was purified by column chromatography to give ethyl 4-[5-fluoro-2-(methyloxy)-3-(4,4,5,5-tetramethyl-1,3,2-dioxaborolan-2-yl)phenyl]butanoate (D116) (113 mg). MS (ES): C₁₉H₂₈BFO₅ requires 366; found 367.3 (M+H⁺).

### DESCRIPTION FOR D117

### 4-bromo-2-chloro-1-[(1-methylethyl)oxy]benzene (D117)

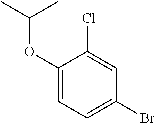

To a solution of 4-bromo-2-chlorophenol (50 g) in N,N-dimethylformamide (DMF) (250 mL) stirred under nitrogen at room temperature was added K₂CO₃ (100 g) and 2-bromopropane (136 mL) in one charge. The reaction mixture was stirred at 85° C. for 16 h. After cooling the reaction, the reaction mixture was filtered, the solvent of the filtrate was removed in vacuo. The residue was dissolved in diethyl ether (300 mL), washed with water (6*100 mL), the organic phase was dried over MgSO₄ and concentrated to give 4-bromo-2-chlorophenyl 1-methylethyl ether (D117) (56 g) as a yellow oil. δH (CDCl₃, 400 MHz): 1.37 (6H, d), 4.52 (1H, m), 6.82 (1H, d), 7.29 (1H, m), 7.50 (1H, d).

### DESCRIPTION FOR D118

### 2-{3-chloro-4-[(1-methylethyl)oxy]phenyl}-4,4,5,5-tetramethyl-1,3,2-dioxaborolane (D118)

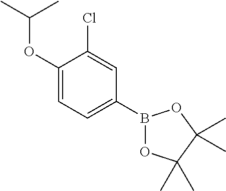

To a suspension of 4-bromo-2-chloro-1-[(1-methylethyl)oxy]benzene (D117) (10 g), 4,4,4′,4′,5,5,5′,5′-octamethyl-2,2′-bi-1,3,2-dioxaborolane (15.26 g) and potassium acetate (15.73 g) in N,N-dimethylformamide (DMF) (150 mL) stirred under nitrogen at room temperature was added PdCl₂(dppf)-CH₂Cl₂ adduct (1.964 g). The reaction mixture was stirred at 80° C. overnight. After cooling the reaction, the reaction mixture was concentrated in vacuo, the residue was diluted with ethyl acetate and filtered through celite, the filtrate was washed with water and brine, the organic phase was dried over anhydrous Na₂SO₄. After removing the solvent, the residue was purified by column chromatography to give 2-{3-chloro-4-[(1-methylethyl)oxy]phenyl}-4,4,5,5-tetramethyl-1,3,2-dioxaborolane (D118) (11.8 g). MS (ES): C₁₅H₂₂BClO₃ requires 296; found 297.1 (M+H⁺).

### DESCRIPTION FOR D119

### 2-[(1-methylethyl)oxy]-5-(1,3-thiazol-2-yl)benzonitrile (D119)

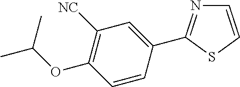

To a suspension of 2-[(1-methylethyl)oxy]-5-(4,4,5,5-tetramethyl-1,3,2-dioxaborolan-2-yl)benzonitrile (D103) (109 mg), 2-bromo-1,3-thiazole (93 mg) and cesium carbonate (148 mg) in acetonitrile (3 mL)/water (0.750 mL) stirred under nitrogen was added PdCl₂(dppf)-CH₂Cl₂ adduct (31.0 mg). The reaction vessel was sealed and heated under microwave at 120° C. for 1 h. After cooling the reaction, the reaction mixture was diluted with ethyl acetate, filtered through celite. The filtrate was partitioned between ethyl acetate and water. The organic phase was dried over sodium sulphate and evaporated in vacuo to give the crude product, which was purified by column chromatography to afford 2-[(1-methylethyl)oxy]-5-(1,3-thiazol-2-yl)benzonitrile (D119) (50 mg). MS (ES): C₁₃H₁₂N₂OS requires 244; found 245.1 (M+H⁺).

### DESCRIPTION FOR D120

### 5-(5-bromo-1,3-thiazol-2-yl)-2-[(1-methylethyl)oxy]benzonitrile (D120)

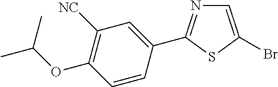

To a solution of 2-[(1-methylethyl)oxy]-5-(1,3-thiazol-2-yl)benzonitrile (D119) (215 mg) and sodium acetate (144 mg) in acetic acid (8 mL) stirred at room temperature was added a solution of Br₂ (0.045 mL) in acetic acid (1 mL) dropwise. The reaction mixture was stirred at 20° C. until start material was consumed completely. The reaction mixture was basified with 2M NaOH, then diluted with ethyl acetate. The mixture was washed with water and brine. The organic phase was dried over anhydrous sodium sulphate. After concentration, the crude product 5-(5-bromo-1,3-thiazol-2-yl)-2-[(1-methylethyl)oxy]benzonitrile (D120) (320 mg) was used for next step without further purification. δH (CDCl₃, 600 MHz): 1.36 (6H, d), 4.65 (1H, m), 6.95 (1H, d), 7.64 (1H, s), 7.92 (1H, d), 7.97 (1H, dd).

### DESCRIPTION FOR D121

### ethyl 4-[3-(2-{3-cyano-4-[(1-methylethyl)oxy]phenyl}-1,3-thiazol-5-yl)-2-ethylphenyl]butanoate (D121)

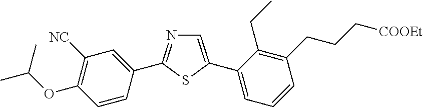

To a suspension of 5-(5-bromo-1,3-thiazol-2-yl)-2-[(1-methylethyl)oxy]benzonitrile (D120) (110 mg), ethyl 4-[2-ethyl-3-(4,4,5,5-tetramethyl-1,3,2-dioxaborolan-2-yl)phenyl]butanoate (D94) (98 mg) and tripotassium phosphate (150 mg) in N,N-dimethylformamide (DMF) (4 mL)/water (1.000 mL) stirred under nitrogen was added Pd(Ph₃P)₄ (32.7 mg). The reaction vessel was sealed and heated under microwave at 120° C. for 20 min. After cooling the reaction, The reaction mixture was diluted with ethyl acetate, filtered through celite. The filtrate was partitioned between ethyl acetate and water. The organic phase was dried over sodium sulphate and evaporated in vacuo to afford the crude product ethyl 4-[3-(2-{3-cyano-4-[(1-methylethyl)oxy]phenyl}-1,3-thiazol-5-yl)-2-ethylphenyl]butanoate (D121) (320 mg). The crude product was used for next step without further purification. MS (ES): C₂₇H₃₀N₂O₃S requires 462; found 463.3 (M+H⁺).

### DESCRIPTION FOR D122

### ethyl 4-[3-(2-{3-cyano-4-[(1-methylethyl)oxy]phenyl}-1,3-thiazol-5-yl)-5-fluoro-2-(methyloxy)phenyl]butanoate (D122)

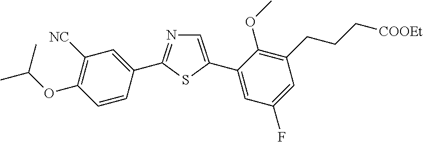

To a suspension of 5-(5-bromo-1,3-thiazol-2-yl)-2-[(1-methylethyl)oxy]benzonitrile (D120) (110 mg), ethyl 4-[5-fluoro-2-(methyloxy)-3-(4,4,5,5-tetramethyl-1,3,2-dioxaborolan-2-yl)phenyl]butanoate (D116) (74.8 mg) and tripotassium phosphate (99 mg) in 1,2-Dimethoxyethane (DME) (2 mL)/water (0.4.000 mL) stirred under nitrogen was added Pd(Ph₃P)₄ (21.45 mg). The reaction vessel was sealed and heated under microwave at 130° C. for 15 min. After cooling the reaction, The reaction mixture was diluted with ethyl acetate, filtered through celite. The filtrate was partitioned between ethyl acetate and water. The organic phase was dried over sodium sulphate and evaporated in vacuo to afford the crude product ethyl 4-[3-(2-{3-cyano-4-[(1-methylethyl)oxy]phenyl}-1,3-thiazol-5-yl)-5-fluoro-2-(methyloxy)phenyl]butanoate (D122) (280 mg). The crude product was used for next step without further purification. MS (ES): C₂₆H₂₇FN₂O₄S requires 482; found 483.2 (M+H⁺).

### DESCRIPTION FOR D123

### 2-{3-chloro-4-[(1-methylethyl)oxy]phenyl}-1,3-thiazole (D123)

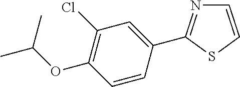

To a suspension of 2-{3-chloro-4-[(1-methylethyl)oxy]phenyl}-4,4,5,5-tetramethyl-1,3,2-dioxaborolane (D118) (3 g), 2-bromo-1,3-thiazole (1.659 g) and Cs₂CO₃ (3.95 g) in 1,2-dimethoxyethane (DME) (40 mL)/water (10 mL) stirred under nitrogen at room temperature was added PdCl₂(dppf)-CH₂Cl₂ adduct (661 mg) in one charge. The reaction vessel was sealed and heated under microwave at 120° C. for 2 h. After cooling the reaction, the reaction mixture was filtered and the filtrate was partitioned between ethyl acetate (150 mL) and saturated brine (50 mL). The organic phase was dried over sodium sulphate and evaporated in vacuo. The crude product was purified by column chromatography to give 2-{3-chloro-4-[(1-methylethyl)oxy]phenyl}-1,3-thiazole (D123) (1.5 g). MS (ES): C₁₂H₁₂ClNOS requires 253; found 254.1 (M+H⁺).

### DESCRIPTION FOR D124

### 5-bromo-2-{3-chloro-4-[(1-methylethyl)oxy]phenyl}-1,3-thiazole (D124)

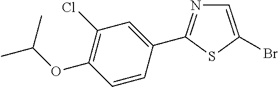

To a solution of 2-{3-chloro-4-[(1-methylethyl)oxy]phenyl}-1,3-thiazole (D123) (1.5 g) and sodium acetate (0.970 g) in acetic acid (10 mL) stirred at room temperature was added a solution of Br₂ (0.31 ml) in acetic acid (1 mL) dropwise during 30 min. The reaction mixture was stirred at 20° C. until start material was consumed completely. The reaction mixture was basified with 2M NaOH. The resulting solution was diluted with ethyl acetate. The mixture was washed with brine. The organic phase was dried over anhydrous sodium sulphate and evaporated in vacuo. The crude product was purified by column chromatography to give 5-bromo-2-{3-chloro-4-[(1-methylethyl)oxy]phenyl}-1,3-thiazole (D124) (1.2 g). MS (ES): C₁₂H₁₁BrClNOS requires 332; found 333.0 (M+H⁺).

### DESCRIPTION FOR D125

### ethyl 4-[3-(2-{3-chloro-4-[(1-methylethyl)oxy]phenyl}-1,3-thiazol-5-yl)-2-ethylphenyl]butanoate (D125)

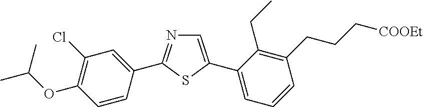

To a suspension of 5-bromo-2-{3-chloro-4-[(1-methylethyl)oxy]phenyl}-1,3-thiazole (D124) (92 mg), ethyl 4-[2-ethyl-3-(4,4,5,5-tetramethyl-1,3,2-dioxaborolan-2-yl)phenyl]butanoate (D94) (115 mg) and tripotassium phosphate (106 mg) in acetonitrile (6 mL) and water (2 mL) stirred under nitrogen was added Pd(Ph₃P)₄ (32.1 mg). The reaction vessel was sealed and heated under microwave at 120° C. for 15 min. After cooling the reaction, The reaction mixture was diluted with ethyl acetate, filtered through celite. The filtrate was partitioned between ethyl acetate and water. The organic phase was dried over sodium sulphate. After concentration, the crude product was purified by column chromatography to afford ethyl 4-[3-(2-{3-chloro-4-[(1-methylethyl)oxy]phenyl}-1,3-thiazol-5-yl)-2-ethylphenyl]butanoate (D125) (113 mg). MS (ES): C₂₆H₃₀ClNO₃S requires 471; found 472.2 (M+H⁺).

### DESCRIPTION FOR D126

### 5-[5-(2-ethyl-3-formylphenyl)-1,3-thiazol-2-yl]-2-[(1-methylethyl)oxy]benzonitrile (D126)

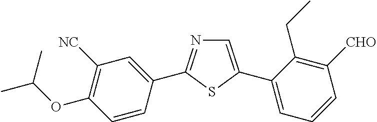

To a solution of 5-(5-bromo-1,3-thiazol-2-yl)-2-[(1-methylethyl)oxy]benzonitrile (D120) (500 mg), 2-ethyl-3-(4,4,5,5-tetramethyl-1,3,2-dioxaborolan-2-yl)benzaldehyde (443 mg) and tripotassium phosphate (657 mg) in N,N-dimethylformamide (DMF) (12 mL) and water (2 mL) stirred under nitrogen at room temperature was added Pd(Ph₃P)₄ (179 mg) in one charge. The reaction vessel was sealed and heated under microwave at 120° C. for 15 min. After cooling the reaction, the reaction mixture was filtered and the filtrate was partitioned between ethyl acetate (250 mL) and saturated brine (50 mL). The organic phase was dried over sodium sulphate and evaporated in vacuo. The crude product was purified by column chromatography to give 5-[5-(2-ethyl-3-formylphenyl)-1,3-thiazol-2-yl]-2-[(1-methylethyl)oxy]benzonitrile (D126) (500 mg) as a brown oil. MS (ES): C₂₂H₂₀N₂O₂S requires 376; found 377.1 (M+H⁺).

### DESCRIPTION FOR D127

### 3-(2-{3-chloro-4-[(1-methylethyl)oxy]phenyl}-1,3-thiazol-5-yl)-2-ethylbenzaldehyde (D127)

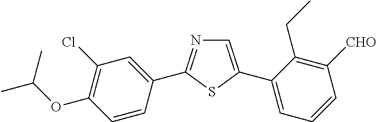

To a solution of 5-bromo-2-{3-chloro-4-[(1-methylethyl)oxy]phenyl}-1,3-thiazole (D124) (500 mg), 2-ethyl-3-(4,4,5,5-tetramethyl-1,3,2-dioxaborolan-2-yl)benzaldehyde (430 mg) and tripotassium phosphate (638 mg) in N,N-dimethylformamide (DMF) (12 mL) and water (2 mL) stirred under nitrogen at room temperature was added Pd(Ph₃P)₄ (174 mg) in one charge. The reaction vessel was sealed and heated under microwave at 120° C. for 15 min. After cooling the reaction, the reaction mixture was filtered and the filtrate was partitioned between ethyl acetate (250 mL) and saturated brine (50 mL). The organic phase was dried over sodium sulphate and evaporated in vacuo. The crude product was purified by column chromatography to give 3-(2-{3-chloro-4-[(1-methylethyl)oxy]phenyl}-1,3-thiazol-5-yl)-2-ethylbenzaldehyde (D127) (500 mg) as a brown oil. MS (ES): C₂₁H₂₀ClNO₂S requires 385; found 386.0 (M+H⁺).

### DESCRIPTION FOR D128

### 5-(5-{2-ethyl-3-[(E)-2-(methyloxy)ethenyl]phenyl}-1,3-thiazol-2-yl)-2-[(1-methylethyl)oxy]benzonitrile (D128)

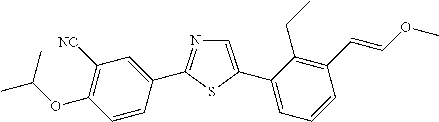

To a solution of 5-(5-bromo-1,3-thiazol-2-yl)-2-[(1-methylethyl)oxy]benzonitrile (D120) (500 mg), 2-{2-ethyl-3-[(E)-2-(methyloxy)ethenyl]phenyl}-4,4,5,5-tetramethyl-1,3,2-dioxaborolane (401 mg) and tripotassium phosphate (591 mg) in N,N-dimethylformamide (DMF) (12 mL) and water (2 mL) stirred under nitrogen at room temperature was added Pd(Ph₃P)₄ (161 mg) in one charge. The reaction vessel was sealed and heated under microwave at 120° C. for 15 min. After cooling the reaction, the reaction mixture was filtered and the filtrate was partitioned between ethyl acetate (250 mL) and saturated brine (50 mL). The organic phase was dried over sodium sulphate and evaporated in vacuo. The crude product was purified by column chromatography to give 5-(5-{2-ethyl-3-[(E)-2-(methyloxy)ethenyl]phenyl}-1,3-thiazol-2-yl)-2-[(1-methylethyl)oxy]benzonitrile (D128) (548 mg) as a brown oil. MS (ES): C₂₄H₂₄N₂O₂S requires 404; found 405.2 (M+H⁺).

### DESCRIPTION FOR D129

### 5-{5-[2-ethyl-3-(2-oxoethyl)phenyl]-1,3-thiazol-2-yl}-2-[(1-methylethyl)oxy]benzonitrile (D129)

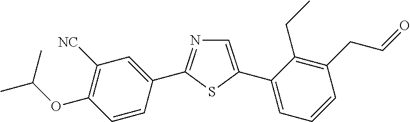

To a mixture of 5-(5-{2-ethyl-3-[(E)-2-(methyloxy)ethenyl]phenyl}-1,3-thiazol-2-yl)-2-[(1-methylethyl)oxy]benzonitrile (D128) (550 mg) in tetrahydrofuran (THF) (20 mL) under nitrogen was added HCl (1.360 mL). The reaction was heated to 75° C. for 6 h. The mixture was concentrated to give the crude product 5-{5-[2-ethyl-3-(2-oxoethyl)phenyl]-1,3-thiazol-2-yl}-2-[(1-methylethyl)oxy]benzonitrile (D129) (531 mg) as a brown oil. MS (ES): C₂₃H₂₂N₂O₂S requires 390; found 391.0 (M+H⁺).

### DESCRIPTION FOR D130

### 2-{3-chloro-4-[(1-methylethyl)oxy]phenyl}-5-{2-ethyl-3-[(E)-2-(methyloxy)ethenyl]phenyl}-1,3-thiazole (D130)

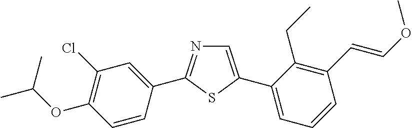

To a solution of 5-bromo-2-{3-chloro-4-[(1-methylethyl)oxy]phenyl}-1,3-thiazole (D124) (500 mg), 2-{2-ethyl-3-[(E)-2-(methyloxy)ethenyl]phenyl}-4,4,5,5-tetramethyl-1,3,2-dioxaborolane (530 mg) and tripotassium phosphate (651 mg) in N,N-dimethylformamide (DMF) (12 mL) and water (2 mL) stirred under nitrogen at room temperature was added Pd(Ph₃P)₄ (177 mg) in one charge. The reaction vessel was sealed and heated under microwave at 120° C. for 15 min. After cooling the reaction, the reaction mixture was filtered and the filtrate was partitioned between ethyl acetate (250 mL) and saturated brine (50 mL). The organic phase was dried over sodium sulphate and evaporated in vacuo. The crude product was purified by column chromatography to give 2-{3-chloro-4-[(1-methylethyl)oxy]phenyl}-5-{2-ethyl-3-[(E)-2-(methyloxy)ethenyl]phenyl}-1,3-thiazole (D130) (500 mg) as a brown oil. MS (ES): C₂₃H₂₄ClNO₂S requires 413; found 414.2 (M+H⁺).

### DESCRIPTION FOR D131

### [3-(2-{3-chloro-4-[(1-methylethyl)oxy]phenyl}-1,3-thiazol-5-yl)-2-ethylphenyl]acetaldehyde (D131)

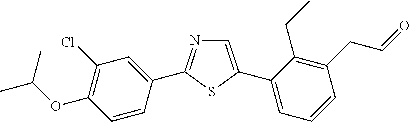

To a mixture of 2-{3-chloro-4-[(1-methylethyl)oxy]phenyl}-5-{2-ethyl-3-[(E)-2-(methyloxy)ethenyl]phenyl}-1,3-thiazole (D130) (500 mg) in tetrahydrofuran (THF) (100 mL) under nitrogen was added HCl (0.805 mL). The reaction was heated to 75° C. for 3 h. The mixture was concentrated to give the crude product [3-(2-{3-chloro-4-[(1-methylethyl)oxy]phenyl}-1,3-thiazol-5-yl)-2-ethylphenyl]acetaldehyde (D131) (483 mg) as a brown oil. MS (ES): C₂₂H₂₂ClNO₂S requires 399; found 400.1 (M+H⁺).

### DESCRIPTION FOR D132

### methyl N-{2-[3-(2-{3-cyano-4-[(1-methylethyl)oxy]phenyl}-1,3-thiazol-5-yl)-2-ethylphenyl]ethyl}-N-methylglycinate (D132)

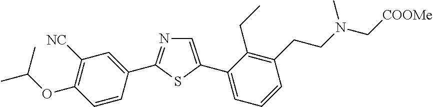

To a solution of 5-{5-[2-ethyl-3-(2-oxoethyl)phenyl]-1,3-thiazol-2-yl}-2-[(1-methylethyl)oxy]benzonitrile (D129) (150 mg), acetic acid (0.044 mL) and sodium acetate (63 mg) in ethanol (20.00 mL) stirred under nitrogen at room temperature was added methyl N-methylglycinate hydrochloride (161 mg) in one charge. The reaction mixture was stirred at room temperature for 30 min, then the solvent was evaporated in vacuo. The residue was dissolved in dichloromethane (DCM) (20 mL), sodium triacetoxyborohydride (204 mg) was added to the mixture. The reaction mixture was stirred at room temperature overnight. The reaction mixture was partitioned between ethyl acetate (100 mL) and saturated brine (30 mL). The organic phase was dried over sodium sulphate and evaporated in vacuo to give the crude product methyl N-{2-[3-(2-{3-cyano-4-[(1-methylethyl)oxy]phenyl}-1,3-thiazol-5-yl)-2-ethylphenyl]ethyl}-N-methylglycinate (D132) (183 mg). The crude product was used for next step without further purification. MS (ES): C₂₇H₃₁N₃O₃S requires 477; found 478.2 (M+H⁺).

### DESCRIPTION FOR D133

### methyl N-{[3-(2-{3-chloro-4-[(1-methylethyl)oxy]phenyl}-1,3-thiazol-5-yl)-2-ethylphenyl]methyl}-N-methylglycinate (D133)

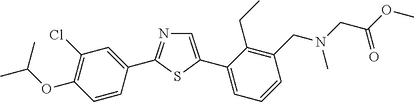

To a solution of 3-(2-{3-chloro-4-[(1-methylethyl)oxy]phenyl}-1,3-thiazol-5-yl)-2-ethylbenzaldehyde (D127) (120 mg), acetic acid (0.036 mL) and sodium acetate (51.0 mg) in ethanol (20.00 mL) stirred under nitrogen at room temperature was added methyl N-methylglycinate hydrochloride (174 mg) in one charge. The reaction mixture was stirred at room temperature for 30 min, then the solvent was evaporated in vacuo. The residue was dissolved in dichloromethane (DCM) (20 mL), sodium triacetoxyborohydride (165 mg) was added to the mixture. The reaction mixture was stirred at room temperature overnight. The reaction mixture was partitioned between ethyl acetate (100 mL) and saturated brine (30 mL). The organic phase was dried over sodium sulphate and evaporated in vacuo to give the crude product methyl N-{[3-(2-{3-chloro-4-[(1-methylethyl)oxy]phenyl}-1,3-thiazol-5-yl)-2-ethylphenyl]methyl}-N-methylglycinate (D133) (100 mg) as a brown oil. The crude product was used for next step without further purification. MS (ES): C₂₅H₂₉ClN₂O₃S requires 472; found 473.2 (M+H⁺).

### DESCRIPTION FOR D134

### methyl N-{[3-(2-{3-cyano-4-[(1-methylethyl)oxy]phenyl}-1,3-thiazol-5-yl)-2-ethylphenyl]methyl}-N-methylglycinate (D134)

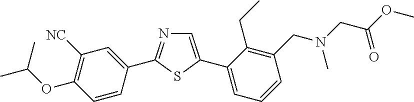

To a solution of 5-[5-(2-ethyl-3-formylphenyl)-1,3-thiazol-2-yl]-2-[(1-methylethyl)oxy]benzonitrile (D126) (120 mg), acetic acid (0.036 mL) and sodium acetate (52.3 mg) in ethanol (20.00 mL) stirred under nitrogen at room temperature was added methyl N-methylglycinate hydrochloride (178 mg) in one charge. The reaction mixture was stirred at room temperature for 30 min, then the solvent was evaporated in vacuo. The residue was dissolved in dichloromethane (DCM) (20 mL), sodium triacetoxyborohydride (169 mg) was added to the mixture. The reaction mixture was stirred at room temperature overnight. The reaction mixture was partitioned between ethyl acetate (100 mL) and saturated brine (30 mL). The organic phase was dried over sodium sulphate and evaporated in vacuo to give the crude product methyl N-{[3-(2-{3-cyano-4-[(1-methylethyl)oxy]phenyl}-1,3-thiazol-5-yl)-2-ethylphenyl]methyl}-N-methylglycinate (D134) (148 mg) as a brown oil. The crude product was used for next step without further purification. MS (ES): C₂₆H₂₉N₃O₃S requires 463; found 464.1 (M+H⁺).

### DESCRIPTION FOR D135

### methyl N-{2-[3-(2-{3-chloro-4-[(1-methylethyl)oxy]phenyl}-1,3-thiazol-5-yl)-2-ethylphenyl]ethyl}-N-methylglycinate (D135)

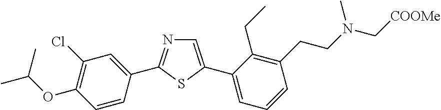

To a solution of [3-(2-{3-chloro-4-[(1-methylethyl)oxy]phenyl}-1,3-thiazol-5-yl)-2-ethylphenyl]acetaldehyde (D131) (160 mg), acetic acid (0.046 mL) and sodium acetate (65.6 mg) in ethanol (20.00 mL) stirred under nitrogen at room temperature was added methyl N-methylglycinate hydrochloride (168 mg) in one charge. The reaction mixture was stirred at room temperature for 30 min, then the solvent was evaporated in vacuo. The residue was dissolved in dichloromethane (DCM) (20 mL), sodium triacetoxyborohydride (165 mg) was added to the mixture. The reaction mixture was stirred at room temperature overnight. The reaction mixture was partitioned between ethyl acetate (100 mL) and saturated brine (30 mL). The organic phase was dried over sodium sulphate and evaporated in vacuo to give the crude product methyl N-{2-[3-(2-{3-chloro-4-[(1-methylethyl)oxy]phenyl}-1,3-thiazol-5-yl)-2-ethylphenyl]ethyl}-N-methylglycinate (D135) (195 mg) as a brown oil. The crude product was used for next step without further purification. MS (ES): C₂₆H₃₁ClN₂O₃S requires 486; found 487.2 (M+H⁺).

### DESCRIPTION FOR D136

### 5-[5-(2-chloro-3-ethyl-4-pyridinyl)-1,3-thiazol-2-yl]-2-[(1-methylethyl)oxy]benzonitrile (D136)

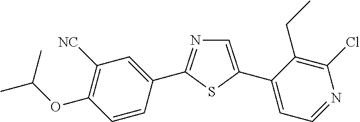

To a suspension of 5-(5-bromo-1,3-thiazol-2-yl)-2-[(1-methylethyl)oxy]benzonitrile (D120) (150 mg), (2-chloro-3-ethyl-4-pyridinyl)boronic acid (126 mg) and Cs₂CO₃ (403 mg) in 1,2-dimethoxyethane (DME) (2 mL)/water (0.5 mL) stirred under nitrogen at room temperature was added PdCl₂(dppf)-CH₂Cl₂ adduct (50.5 mg). The reaction mixture was sealed and heated under microwave at 120° C. for 30 min. After cooling the reaction, the mixture was diluted with ethyl acetate and filtered through silical gel. The filtrate was washed with aqueous saturated ammonium chloride and saturated brine. The organic phase was collected and dried over anhydrous sodium sulphate. The solvent was removed in vacuo to give the crude product, which was purified by column chromatography to afford 5-[5-(2-chloro-3-ethyl-4-pyridinyl)-1,3-thiazol-2-yl]-2-[(1-methylethyl)oxy]benzonitrile (D136) (250 mg). MS (ES): C₂₀H₁₈ClN₃OS requires 383; found 384.1 (M+H⁺).

### DESCRIPTION 137

### 1,1-dimethylethyl (2E)-3-(3-cyanophenyl)-2-propenoate (D137)

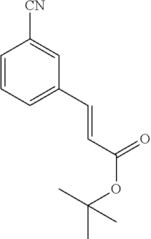

A mixture of 3-bromobenzonitrile (5 g, 27.5 mmol) 1,1-dimethylethyl 2-propenoate (4.39 ml, 30.2 mmol), bis(tri-t-butylphosphine)palladium(0) (0.702 g, 1.373 mmol), and dicyclohexyl(methyl)amine (1.165 ml, 5.49 mmol) in 1,4-dioxane (60 ml) were heated at 70° C. for 17 hrs. The cooled reaction mixture was diluted with ethyl acetate, filtered through celite and the filtrate evaporated to give a browncoloured oil (6.2 g) which was loaded on top of Silica Biotage SP4; 40+M (40×150 mm) cartridge and was purified by eluting with a gradient of 6-20% of Ethyl Acetate over 10 column volumes. The UV active fraction were analysed by TLC and pure fractions were combined to yield an orange solid which was analysed by LCMS (2.13 g)

¹H NMR (400 MHz, CDCl₃) δ ppm 7.78 (t, J=1.5 Hz, 1H), 7.72 (dt, J=7.5, 1.5 Hz, 1H), 7.64 (dt, J=7.5, 1.5 Hz, 1H), 7.54 (d, J=16.0 Hz, 1H), 7.50 (t, J=7.5 Hz, 1H), 6.42 (d, J=16.0 Hz, 1H), 1.54 (s, 9H)

### DESCRIPTION 138

### 1,1-dimethylethyl 3-(3-cyanophenyl)propanoate (D138)

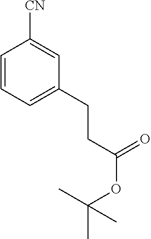

A solution of 1,1-dimethylethyl (2E)-3-(3-cyanophenyl)-2-propenoate (D137; 2.12 g, 9.25 mmol) in ethanol (40 ml) was hydrogenated over palladium on carbon (0.08 g, 0.752 mmol) over 5 hr. A further portion of palladium on carbon (0.08 g, 0.254 mmol) was added and the reaction mixture was stirred under hydrogen for a further 7 hours. A further portion of palladium on carbon (0.08 g, 0.254 mmol) was added and the reaction mixture was stirred under hydrogen for a further 7 hours. Palladium on Carbon (0.08 g, 0.752 mmol) was added and the reaction mixture was stirred under continuous supply of hydrogen for 7 hrs. The reaction mixture was filtered through celite and the filtrate evaporated to give the title compound as a colourless oil (1.97 g)

¹H NMR (400 MHz, CDCl₃) δ ppm 6.97-7.53 (m, 4H), 2.94 (t, J=7.5 Hz, 2H), 2.55 (t, J=7.5 Hz, 2H), 1.41 (s, 9H)

### DESCRIPTION 139

### 1,1-dimethylethyl 3-{3-[(hydroxyamino)(imino)methyl]phenyl}propanoate (D139)

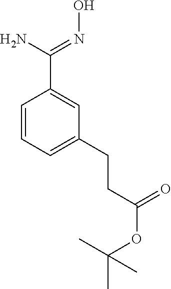

A mixture of 1,1-dimethylethyl 3-(3-cyanophenyl)propanoate (D138); 1.96 g, 8.47 mmol), hydroxylamine hydrochloride (1.178 g, 16.95 mmol), and sodium bicarbonate (2.85 g, 33.9 mmol) in ethanol (30 ml) were heated at 60° C. for 20 hr. The reaction mixture was filtered and the filtrate evaporated to yield a colourless oil which was dissolved in ethyl acetate (25 ml) and washed with water (3×10 ml). The organic layer was dried over anhydrous sodium sulphate and evaporated to give the title compound as an oil (2.18 g)

MS: (+ve ion electrospray) m/z 265 [MH⁺];

### DESCRIPTION 140

### 1,1-dimethylethyl 3-[3-(5-{3-chloro-4-[(1-methylethyl)oxy]phenyl}-1,2,4-oxadiazol-3-yl)phenyl]propanoate (D140)

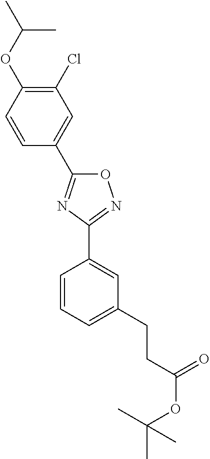

1,1-dimethylethyl 3-{3-[(hydroxyamino)(imino)methyl]phenyl}propanoate (D139; 2.18 g, 8.25 mmol) in dry DMF (15 ml) was cooled in ice bath and was treated with triethylamine (1.61 ml, 11.55 mmol) followed by the dropwise addition of a solution of 3-chloro-4-[(1-methylethyl)oxy]benzoyl chloride (2.31 g, 9.90 mmol) in dry DMF (10 ml). The yellow coloured reaction mixture was stirred at room temperature for 1 hr then heated at 120° C. for 2 hrs. The reaction mixture was partitioned between ethyl acetate (3×150 ml) and water (200 ml). The combined organic layers were washed with water (3×200 ml), dried over anhydrous sodium sulphate and evaporated to yield a brown oil which was purified by chromatography on silica. Eluting with a gradient of 10-30% ethyl acetate in cyclohexane gave the title compound as a white solid (1.94 g)

MS: (+ve ion electrospray) m/z 443 [MH⁺];

### EXAMPLE-1

### 3-[3-(5-{3-chloro-4-[(1-methylethyl)oxy]phenyl}-1,2,4-oxadiazol-3-yl)-2-methylphenyl]propanoic acid (E1)

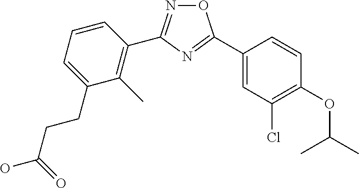

Sodium hydroxide (59 mg) was added to a solution of ethyl 3-[3-(5-{3-chloro-4-[(1-methylethyl)oxy]phenyl}-1,2,4-oxadiazol-3-yl)-2-methylphenyl]propanoate (D3) (126 mg) in ⁱPrOH (4 ml) and water (4 ml). The resulting mixture was stirred at 65° C. for 2 hours. Then 2 M HCl solution was added until pH was about 6. The organic phase was washed with water (10 mL), dried over sodium sulphate and evaporated in vacuo. The precipitate was purified by Mass Directed Auto Prep to afford 3-[3-(5-{3-chloro-4-[(1-methylethyl)oxy]phenyl}-1,2,4-oxadiazol-3-yl)-2-methylphenyl]propanoic acid (E1) (13 mg) as a white solid. δH (CDCl₃, 400 MHz): 1.46 (6H, d), 2.41 (3H, s), 2.74 (2H, t), 3.06 (2H, t), 4.71-4.73 (1H, m), 7.06 (1H, d), 7.27 (1H, d), 7.95 (2H, d), 8.06-8.08 (1H, dd), 8.25 (1H, d). MS (ES): C₂₁H₂₁ClN₄O₄ requires 400; found 401.2 (M+H⁺).

### EXAMPLE-2

### 4-[3-(5-{3-chloro-4-[(1-methylethyl)oxy]phenyl}-1,2,4-oxadiazol-3-yl)-2-methylphenyl]butanoic acid (E2)

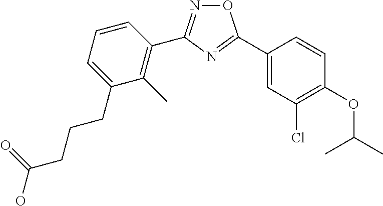

Sodium hydroxide (38 mg) was added to a solution of ethyl 4-[3-(5-{3-chloro-4-[(1-methylethyl)oxy]phenyl}-1,2,4-oxadiazol-3-yl)-2-methylphenyl]butanoate (D4) (141 mg) in ⁱPrOH (4 ml) and water (4 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-chloro-4-[(1-methylethyl)oxy]phenyl}-1,2,4-oxadiazol-3-yl)-2-methylphenyl]butanoic acid (E2) (41 mg) as a white solid. δH (CDCl₃, 400 MHz): 1.46 (6H, d), 2.01-2.04 (2H, m), 2.40 (3H, s), 2.49 (2H, t), 2.77 (2H, t), 4.71-4.73 (1H, m), 7.06 (1H, d), 7.27 (1H, d), 7.91 (2H, d), 8.06 (1H, dd), 8.25 (1H, d). MS (ES): C₂₂H₂₃ClN₂O₄ requires 414; found 415.2 (M+H⁺).

### EXAMPLE-3

### (2R)-3-[3-(5-{5-chloro-6-[(1-methylethyl)oxy]-3-pyridinyl}-1,2,4-oxadiazol-3-yl)-2-methylphenyl]-2-methylpropanoic acid (E3)

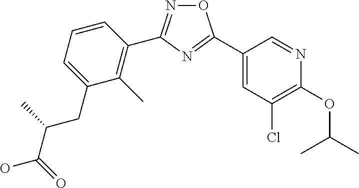

Sodium hydroxide (39 mg) was added to a solution of methyl (2R)-3-[3-(5-{5-chloro-6-[(1-methylethyl)oxy]-3-pyridinyl}-1,2,4-oxadiazol-3-yl)-2-methylphenyl]-2-methylpropanoate (146 mg) in ⁱPrOH (4 ml) and water (4 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford (2R)-3-[3-(5-{5-chloro-6-[(1-methylethyl)oxy]-3-pyridinyl}-1,2,4-oxadiazol-3-yl)-2-methylphenyl]-2-methylpropanoic acid (14 mg) as a white solid. δH (CDCl₃, 400 MHz): 1.27 (3H, d), 1.46 (6H, d), 2.42 (3H, s), 2.69-2.88 (2H, m), 3.17-3.21 (1H, m), 5.50 (1H, m), 7.29 (1H, d), 7.92 (2H, dd), 8.40 (1H, d), 8.88 (1H, dd). MS (ES): C₂₁H₂₂N₃O₄ requires 415; found 416.2 (M+H⁺).

### EXAMPLE-4

### 4-[3-(5-{5-chloro-6-[(1-methylethyl)oxy]-3-pyridinyl}-1,2,4-oxadiazol-3-yl)-2-methylphenyl]butanoic acid (E4)

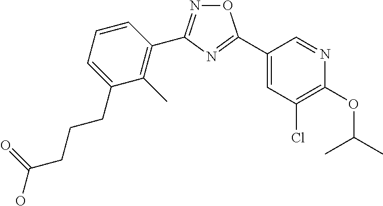

Sodium hydroxide (96 mg) was added to a solution of ethyl 4-[3-(5-{5-chloro-6-[(1-methylethyl)oxy]-3-pyridinyl}-1,2,4-oxadiazol-3-yl)-2-methylphenyl]butanoate (231 mg) in ⁱPrOH (4 ml) and water (4 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{5-chloro-6-[(1-methylethyl)oxy]-3-pyridinyl}-1,2,4-oxadiazol-3-yl)-2-methylphenyl]butanoic acid (13 mg) as a white solid. δH (CDCl₃, 400 MHz): 1.37 (6H, d), 2.01-2.04 (2H, t), 2.40 (3H, s), 2.49 (2H, t), 2.77 (2H, t), 5.50-5.52 (1H, m), 7.38 (1H, d), 8.00 (1H, dd), 8.88 (1H, d), 8.40 (1H, d), 8.88 (1H, d). MS (ES): C₂₁H₂₂ClN₃O₄ requires 415; found 416.2 (M+H⁺).

### EXAMPLE-5

### 3-[3-(5-{3-cyano-4-[(1-methylethyl)oxy]phenyl}-1,2,4-oxadiazol-3-yl)-2-methylphenyl]propanoic acid (E5)

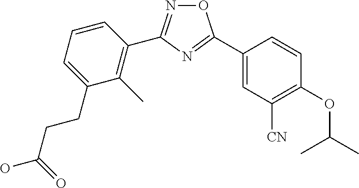

Sodium hydroxide (69 mg) was added to a solution of ethyl 3-[3-(5-{3-cyano-4-[(1-methylethyl)oxy]phenyl}-1,2,4-oxadiazol-3-yl)-2-methylphenyl]propanoate (146 mg) in ⁱPrOH (3 mL) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 3-[3-(5-{3-cyano-4-[(1-methylethyl)oxy]phenyl}-1,2,4-oxadiazol-3-yl)-2-methylphenyl]propanoic acid (36 mg) as a white solid. δH (CDCl₃, 400 MHz): 1.48 (6H, d), 2.42 (3H, s), 2.75 (2H, t), 3.06 (2H, t), 4.79-4.82 (1H, m), 7.12 (1H, d), 7.31 (1H, d), 7.92 (1H, d), 7.96 (1H, s), 8.33-8.36 (1H, dd), 8.44 (1H, d). MS (ES): C₂₂H₂₁N₃O₄ requires 391; found 392.2 (M+H⁺).

### EXAMPLE-6

### 4-[3-(5-{3-cyano-4-[(1-methylethyl)oxy]phenyl}-1,2,4-oxadiazol-3-yl)-2-methylphenyl]butanoic acid (E6)

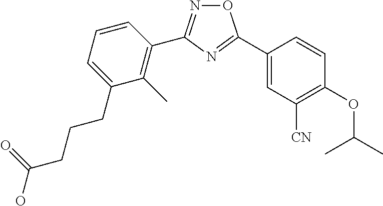

Sodium hydroxide (70 mg) was added to a solution of ethyl 4-[3-(5-{3-cyano-4-[(1-methylethyl)oxy]phenyl}-1,2,4-oxadiazol-3-yl)-2-methylphenyl]butanoate (153 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-cyano-4-[(1-methylethyl)oxy]phenyl}-1,2,4-oxadiazol-3-yl)-2-methylphenyl]butanoic acid (41 mg) as a white solid. δH (CDCl₃, 400 MHz): 1.49 (6H, d), 1.99-2.03 (2H, m), 2.41 (3H, s), 2.49 (2H, t), 2.77 (2H, t), 4.78-4.82 (1H, m), 7.12 (1H, d), 7.29 (1H, d), 7.90 (1H, d), 7.93 (1H, s), 8.33-8.36 (1H, dd), 8.44 (1H, d). MS (ES): C₂₃H₂₃N₃O₄ requires 405; found 406.2 (M+H⁺).

### EXAMPLE-7

### 4-[4-(5-{3-chloro-4-[(1-methylethyl)oxy]phenyl}-1,2,4-oxadiazol-3-yl)-3-methyl-2-pyridinyl]butanoic acid (E7)

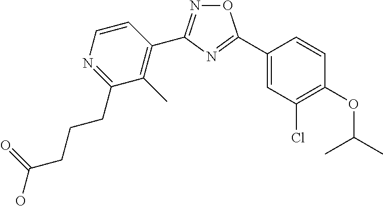

Sodium hydroxide (60 mg) was added to a solution of ethyl 4-[3-(5-{3-chloro-4-[(1-methylethyl)oxy]phenyl}-1,2,4-oxadiazol-3-yl)-2-methylphenyl]butanoate (135 mg) in ⁱPrOH (3 ml) and water (3 ml). The resulting mixture was stirred at 60° C. for 3 hours. Then 2 M HCl solution was added until pH was about 6. The solvent was evaporated, and the residue was extracted by EtOAc (5 ml, three times). The precipitate was purified by Mass Directed Auto Prep to afford 4-[4-(5-{3-chloro-4-[(1-methylethyl)oxy]phenyl}-1,2,4-oxadiazol-3-yl)-3-methyl-2-pyridinyl]butanoic acid (42 mg) as a white solid. δH (CDCl₃, 400 MHz): 1.46 (6H, d), 2.14-2.17 (2H, m), 2.48 (2H, t), 2.69 (3H, s), 3.17 (2H, t), 4.71-4.77 (1H, m), 7.08 (1H, d), 7.87 (1H, d), 8.07 (1H, dd), 8.25 (1H, d), 8.55 (1H, d). MS (ES): C₂₁H₂₂ClN₃O₄ requires 415; found 416.2 (M+H⁺).

### EXAMPLE-8

### 3-[2-chloro-3-(5-{5-chloro-6-[(1-methylethyl)oxy]-3-pyridinyl}-1,2,4-oxadiazol-3-yl)phenyl]propanoic acid (E8)

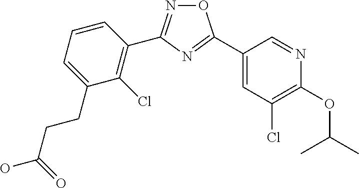

Sodium hydroxide (42 mg) was added to a solution of ethyl 3-[2-chloro-3-(5-{5-chloro-6-[(1-methylethyl)oxy]-3-pyridinyl}-1,2,4-oxadiazol-3-yl)phenyl]propanoate (94 mg) in ⁱPrOH (3 ml) and water (3 ml). The resulting mixture was stirred at 60° C. for 4 hours. Then 2 M HCl solution was added until pH was about 6. The solvent was evaporated, and the residue was extracted by EtOAc (5 ml, three times). The precipitate was purified by Mass Directed Auto Prep to afford 3-[2-chloro-3-(5-{5-chloro-6-[(1-methylethyl)oxy]-3-pyridinyl}-1,2,4-oxadiazol-3-yl)phenyl]propanoic acid (12 mg) as a white solid. δH (CDCl₃, 400 MHz): 1.46 (6H, d), 2.80 (2H, t), 3.21 (2H, t), 5.49-5.52 (1H, m), 7.36-7.39 (1H, dd), 7.49 (2H, d), 7.80 (1H, d), 8.40 (1H, d), 8.88 (1H, d). MS (ES): C₁₉H₁₇Cl₂N₃O₄ requires 421; found 422.2 (M+H⁺).

### EXAMPLE-9

### 4-[2-chloro-3-(5-{5-chloro-6-[(1-methylethyl)oxy]-3-pyridinyl}-1,2,4-oxadiazol-3-yl)phenyl]butanoic acid (E9)

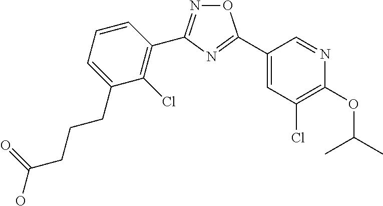

Sodium hydroxide (63 mg) was added to a solution of ethyl 4-[2-chloro-3-(5-{5-chloro-6-[(1-methylethyl)oxy]-3-pyridinyl}-1,2,4-oxadiazol-3-yl)phenyl]butanoate (147 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[2-chloro-3-(5-{5-chloro-6-[(1-methylethyl)oxy]-3-pyridinyl}-1,2,4-oxadiazol-3-yl)phenyl]butanoic acid (74 mg) as a white solid. δH (CDCl₃, 400 MHz): 1.45 (6H, d), 2.04-2.07 (2H, m), 2.47 (2H, t), 2.94 (2H, t), 5.47-5.53 (1H, m), 7.35-7.39 (1H, dd), 7.42-7.45 (1H, dd), 7.76-7.78 (1H, dd), 8.40 (1H, d), 8.88 (1H, d). MS (ES): C₂₀H₁₉Cl₂N₃O₄ requires 435; found 436.2 (M+H⁺).

### EXAMPLE-10

### 3-[2-chloro-3-(5-{3-cyano-4-[(1-methylethyl)oxy]phenyl}-1,2,4-oxadiazol-3-yl)phenyl]propanoic acid (E10)

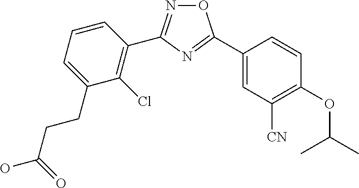

Sodium hydroxide (72 mg) was added to a solution of ethyl 3-[2-chloro-3-(5-{3-cyano-4-[(1-methylethyl)oxy]phenyl}-1,2,4-oxadiazol-3-yl)phenyl]propanoate (159 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 3-[2-chloro-3-(5-{3-cyano-4-[(1-methylethyl)oxy]phenyl}-1,2,4-oxadiazol-3-yl)phenyl]propanoic acid (64 mg) as a white solid. δH (CDCl₃, 400 MHz): 1.49 (6H, d), 2.79 (2H, t), 3.21 (2H, t), 4.79-4.82 (1H, m), 7.13 (1H, d), 7.36-7.39 (1H, dd), 7.49 (1H, d), 7.80 (1H, d), 8.35 (1H, d), 8.44 (1H, s). MS (ES): C₂₁H₁₈ClN₃O₄ requires 411; found 412.2 (M+H⁺).

### EXAMPLE-11

### 4-[2-chloro-3-(5-{3-cyano-4-[(1-methylethyl)oxy]phenyl}-1,2,4-oxadiazol-3-yl)phenyl]butanoic acid (E11)

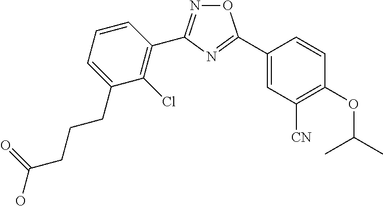

Sodium hydroxide (62 mg) was added to a solution of ethyl 4-[2-chloro-3-(5-{3-cyano-4-[(1-methylethyl)oxy]phenyl}-1,2,4-oxadiazol-3-yl)phenyl]butanoate (142 mg) in ⁱPrOH (3 ml) and water (3 ml). The resulting mixture was stirred at 60° C. for 0.5 hours. Then 2 M HCl solution was added until pH was about 6. The solvent was evaporated, and the residue was extracted by EtOAc (5 ml, three times). The precipitate was purified by Mass Directed Auto Prep to afford 4-[2-chloro-3-(5-{3-cyano-4-[(1-methylethyl)oxy]phenyl}-1,2,4-oxadiazol-3-yl)phenyl]butanoic acid (54 mg) as a white solid. δH (CDCl₃, 400 MHz): 1.48 (6H, d), 2.03-2.07 (2H, m), 2.47 (2H, t), 2.94 (2H, t), 4.78-4.84 (1H, m), 7.13 (1H, d), 7.34-7.38 (1H, dd), 7.42-7.44 (1H, dd), 7.75-7.77 (1H, dd), 8.33-8.36 (1H, dd), 8.44 (1H, d). MS (ES): C₂₂H₂₀ClN₃O₄ requires 425; found 426.2 (M+H⁺).

### EXAMPLE-12

### 6-[2-chloro-3-(5-{3-cyano-4-[(1-methylethyl)oxy]phenyl}-1,2,4-oxadiazol-3-yl)phenyl]hexanoic acid (E12)

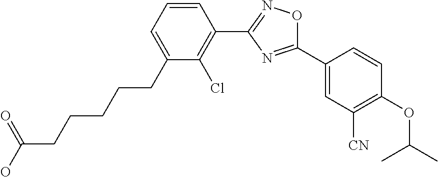

Sodium hydroxide (69 mg) was added to a solution of ethyl 6-[2-chloro-3-(5-{3-cyano-4-[(1-methylethyl)oxy]phenyl}-1,2,4-oxadiazol-3-yl)phenyl]hexanoate (167 mg) in ⁱPrOH (3 ml) and water (3 ml). The resulting mixture was stirred at 50° C. for 3 hours. Then 2 M HCl solution was added until pH was about 6. The solvent was evaporated, and the residue was extracted by EtOAc (5 ml, three times). The precipitate was purified by Mass Directed Auto Prep to afford 6-[2-chloro-3-(5-{3-cyano-4-[(1-methylethyl)oxy]phenyl}-1,2,4-oxadiazol-3-yl)phenyl]hexanoic acid (22 mg) as a white solid. δH (CDCl₃, 400 MHz): 1.48 (6H, d), 1.98-1.99 (2H, m), 2.32 (2H, t), 2.78 (2H, t), 4.71-4.74 (1H, m), 7.05 (1H, d), 7.24-7.28 (1H, dd), 7.32 (1H, dd), 7.65 (1H, d), 8.27 (1H, d), 8.36 (1H, d). MS (ES): C₂₄H₂₄ClN₃O₄ requires 453; found 454.2 (M+H⁺).

### EXAMPLE-13

### 3-[3-(5-{5-chloro-6-[(1-methylethyl)oxy]-3-pyridinyl}-1,2,4-oxadiazol-3-yl)-2-ethylphenyl]propanoic acid (E13)

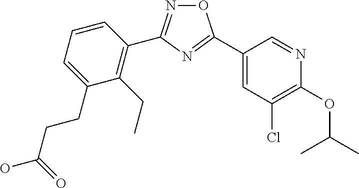

Sodium hydroxide (49 mg) was added to a solution of ethyl 3-[3-(5-{5-chloro-6-[(1-methylethyl)oxy]-3-pyridinyl}-1,2,4-oxadiazol-3-yl)-2-ethylphenyl]propanoate (108 mg) in ⁱPrOH (3 ml) and water (3 ml). The resulting mixture was stirred at 60° C. for 3 hours. Then 2 M HCl solution was added until pH was about 6. The solvent was evaporated, and the residue was extracted by EtOAc (5 ml, three times). The precipitate was purified by Mass Directed Auto Prep to afford 3-[2-chloro-3-(5-{5-chloro-6-[(1-methylethyl)oxy]-3-pyridinyl}-1,2,4-oxadiazol-3-yl)phenyl]propanoic acid (14 mg) as a white solid. δH (CDCl₃, 400 MHz): 1.28 (3H, t), 1.46 (6H, d), 2.73 (2H, t), 3.02 (2H, t), 3.12 (2H, t), 5.48-5.52 (1H, m), 7.28-7.32 (1H, dd), 7.37 (2H, d), 7.76-7.78 (1H, dd), 8.39 (1H, d), 8.88 (1H, d). MS (ES): C₂₁H₂₂ClN₃O₄ requires 415; found 416.2 (M+H⁺).

### EXAMPLE-14

### 4-[3-(5-{5-chloro-6-[(1-methylethyl)oxy]-3-pyridinyl}-1,2,4-oxadiazol-3-yl)-2-ethylphenyl]butanoic acid (E14)

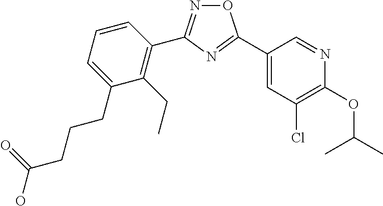

Sodium hydroxide (153 mg) was added to a solution of ethyl 4-[3-(5-{5-chloro-6-[(1-methylethyl)oxy]-3-pyridinyl}-1,2,4-oxadiazol-3-yl)-2-ethylphenyl]butanoate (174 mg) in ⁱPrOH (3 ml) and water (3 ml). The resulting mixture was stirred at 60° C. for 3 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{5-chloro-6-[(1-methylethyl)oxy]-3-pyridinyl}-1,2,4-oxadiazol-3-yl)-2-ethylphenyl]butanoic acid (40 mg) as a white solid. δH (CDCl₃, 400 MHz): 1.21 (3H, t), 1.45 (6H, d), 1.98-2.06 (2H, m), 2.50 (2H, t), 2.81 (2H, t), 2.98-3.03 (2H, m), 5.49-5.52 (1H, m), 7.26-7.36 (2H, m), 7.73-7.75 (1H, dd), 8.39 (1H, d), 8.88 (111, d). MS (ES): C₂₂H₂₄ClN₃O₄ requires 429; found 430.2 (M+H⁺).

### EXAMPLE-15

### 4-[3-(5-{3-cyano-4-[(1-methylethyl)oxy]phenyl}-1,2,4-oxadiazol-3-yl)-2-ethylphenyl]butanoic acid (E15)

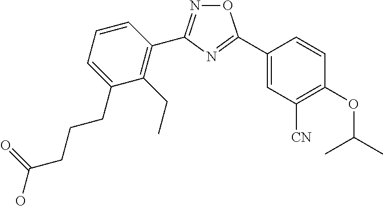

Sodium hydroxide (46 mg) was added to a solution of ethyl 4-[3-(5-{3-cyano-4-[(1-methylethyl)oxy]phenyl}-1,2,4-oxadiazol-3-yl)-2-ethylphenyl]butanoate (152 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-cyano-4-[(1-methylethyl)oxy]phenyl}-1,2,4-oxadiazol-3-yl)-2-ethylphenyl]butanoic acid (46 mg) as a white solid. δH (CDCl₃, 400 MHz): 1.22 (311, t), 1.49 (6H, d), 1.98-2.02 (2H, m), 2.49 (211, t), 2.81 (2H, t), 2.97-3.03 (2H, m), 4.76-4.84 (1H, m), 7.13 (1H, d), 7.29 (1H, d), 7.35 (111, d), 7.72-7.74 (1H, dd), 8.33-8.36 (1H, dd), 8.44 (1H, d). MS (ES): C₂₄H₂₅N₃O₄ requires 419; found 420.2 (M+H⁺).

### EXAMPLE-16

### 3-[3-(5-{3-chloro-4-[(1-methylethyl)oxy]phenyl}-1,2,4-oxadiazol-3-yl)-2-(methyloxy)phenyl]propanoic acid (E16)

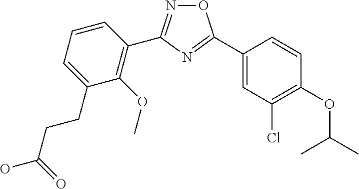

Sodium hydroxide (69 mg) was added to a solution of ethyl 3-[3-(5-{3-chloro-4-[(1-methylethyl)oxy]phenyl}-1,2,4-oxadiazol-3-yl)-2-(methyloxy)phenyl]propanoate (126 mg) in ⁱPrOH (3 ml) and water (3 ml). The resulting mixture was stirred at 60° C. for 2.5 hours. Then 2 M HCl solution was added until pH was about 6. The solvent was evaporated, and the residue was extracted by EtOAc (5 ml, three times). The precipitate was purified by Mass Directed Auto Prep to afford 3-[3-(5-{3-chloro-4-[(1-methylethyl)oxy]phenyl}-1,2,4-oxadiazol-3-yl)-2-(methyloxy)phenyl]propanoic acid (58 mg) as a white solid. δH (CDCl₃, 400 MHz): 1.46 (6H, d), 2.78 (2H, t), 3.10 (2H, t), 3.87 (3H, s), 4.71-4.74 (1H, m), 7.07 (1H, d), 7.20-7.24 (1H, dd), 7.41 (1H, d), 7.95-7.97 (1H, dd), 8.07-8.09 (1H, d), 8.26 (1H, d). MS (ES): C₂₁H₂₁ClN₂O₅ requires 416; found 417.2 (M+H⁺).

### EXAMPLE-17

### 4-[3-(5-{3-chloro-4-[(1-methylethyl)oxy]phenyl}-1,2,4-oxadiazol-3-yl)-2-(methyloxy)phenyl]butanoic acid (E17)

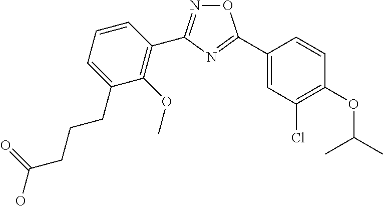

Sodium hydroxide (70 mg) was added to a solution of ethyl 4-[3-(5-{3-chloro-4-[(1-methylethyl)oxy]phenyl}-1,2,4-oxadiazol-3-yl)-2-methylphenyl]butanoate (160 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-chloro-4-[(1-methylethyl)oxy]phenyl}-1,2,4-oxadiazol-3-yl)-2-(methyloxy)phenyl]butanoic acid (55 mg) as a white solid. δH (CDCl₃, 400 MHz): 1.46 (6H, d), 2.02-2.06 (2H, m), 2.45 (2H, t), 2.82 (2H, t), 3.84 (3H, s), 4.71-4.73 (1H, m), 7.06 (1H, d), 7.19-7.23 (1H, dd), 7.37-7.39 (1H, dd), 7.92-7.95 (1H, dd), 8.06-8.08 (111, dd), 8.25 (1H, d). MS (ES): C₂₂H₂₃ClN₂O₅ requires 430; found 431.2 (M+H⁺).

### EXAMPLE-14

### 3-[3-(5-{3-cyano-4-[(1-methylethyl)oxy]phenyl}-1,2,4-oxadiazol-3-yl)-2-(methyloxy)phenyl]propanoic acid (E18)

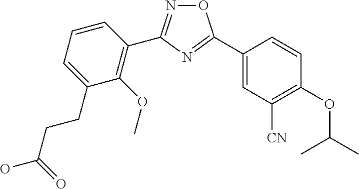

Sodium hydroxide (67 mg) was added to a solution of ethyl ethyl 3-[3-(5-{3-cyano-4-[(1-methylethyl)oxy]phenyl}-1,2,4-oxadiazol-3-yl)-2-(methyloxy)phenyl]propanoate (147 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 3-[3-(5-{3-cyano-4-[(1-methylethyl)oxy]phenyl}-1,2,4-oxadiazol-3-yl)-2-(methyloxy)phenyl]propanoic acid (62 mg) as a white solid. δH (CDCl₃, 400 MHz): 1.48 (6H, d), 2.78 (2H, t), 3.10 (2H, t), 3.87 (3H, s), 4.79-4.82 (1H, m), 7.12 (1H, d), 7.21-7.25 (1H, dd), 7.42 (1H, d), 7.95-7.98 (1H, dd), 8.35-8.37 (1H, dd), 8.45 (1H, d). MS (ES): C₂₂H₂₁N₃O₅ requires 407; found 408.2 (M+H⁺).

### EXAMPLE-19

### 4-[3-(5-{3-cyano-4-[(1-methylethyl)oxy]phenyl}-1,2,4-oxadiazol-3-yl)-2-(methyloxy)phenyl]butanoic acid (E19)

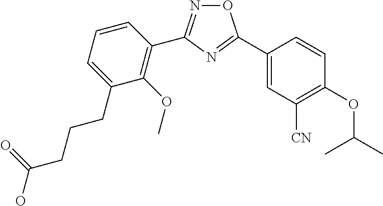

Sodium hydroxide (62 mg) was added to a solution of ethyl 4-[3-(5-{3-cyano-4-[(1-methylethyl)oxy]phenyl}-1,2,4-oxadiazol-3-yl)-2-(methyloxy)phenyl]butanoate (139 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-cyano-4-[(1-methylethyl)oxy]phenyl}-1,2,4-oxadiazol-3-yl)-2-(methyloxy)phenyl]butanoic acid (61 mg) as a white solid. δH (CDCl₃, 400 MHz): 1.49 (6H, d), 2.02-2.06 (2H, m), 2.46 (2H, t), 2.83 (2H, t), 3.85 (3H, s), 4.77-4.84 (1H, m), 7.13 (1H, d), 7.23 (1H, d), 7.39-7.41 (1H, dd), 7.93-7.95 (1H, dd), 8.33-8.36 (1H, dd), 8.44 (1H, d). MS (ES): C₂₃H₂₃N₃O₅ requires 421; found 422.2 (M+H⁺).

### EXAMPLE-20

### 3-[4-(5-{3-cyano-4-[(1-methylethyl)oxy]phenyl}-1,2,4-oxadiazol-3-yl)-2-pyridinyl]propanoic acid (E20)

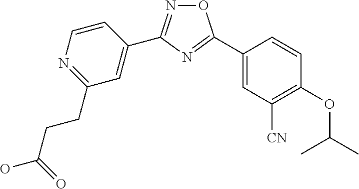

Sodium hydroxide (72 mg) was added to a solution of ethyl 3-[4-(5-{3-cyano-4-[(1-methylethyl)oxy]phenyl}-1,2,4-oxadiazol-3-yl)-2-pyridinyl]propanoate (158 mg) in ⁱPrOH (3 ml) and water (3 ml). The resulting mixture was stirred at 60° C. for 1.5 hours. Then 2 M HCl solution was added until pH was about 6. The solvent was evaporated, and the residue was extracted by EtOAc (5 ml, three times). The precipitate was purified by Mass Directed Auto Prep to afford 3-[4-(5-{3-cyano-4-[(1-methylethyl)oxy]phenyl}-1,2,4-oxadiazol-3-yl)-2-pyridinyl]propanoic acid (11 mg) as a white solid. δH (CDCl₃, 400 MHz): 1.49 (6H, d), 2.48 (2H, t), 3.08 (2H, t), 4.80-4.84 (1H, m), 7.15 (1H, d), 7.95 (1H, d), 7.99 (1H, s), 8.34-8.37 (1H, dd), 8.45 (1H, d), 8.76 (1H, d). MS (ES): C₂₀H₈N₄O₄ requires 378; found 379.2 (M+H⁺).

### EXAMPLE-21

### 4-[4-(5-{3-cyano-4-[(1-methylethyl)oxy]phenyl}-1,2,4-oxadiazol-3-yl)-2-pyridinyl]butanoic acid (E21)

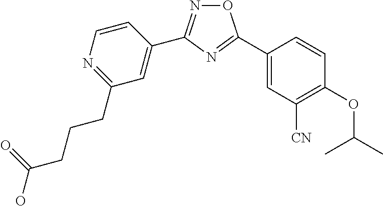

Sodium hydroxide (66 mg) was added to a solution of ethyl 4-[4-(5-{3-cyano-4-[(1-methylethyl)oxy]phenyl}-1,2,4-oxadiazol-3-yl)-2-pyridinyl]butanoate (139 mg) in ⁱPrOH (3 ml) and water (3 ml). The resulting mixture was stirred at 60° C. for 2 hours. Then 2 M HCl solution was added until pH was about 6. The solvent was evaporated, and the residue was extracted by EtOAc (5 ml, three times). The precipitate was purified by Mass Directed Auto Prep to afford 4-[4-(5-{3-cyano-4-[(1-methylethyl)oxy]phenyl}-1,2,4-oxadiazol-3-yl)-2-pyridinyl]butanoic acid (31 mg) as a white solid. δH (CDCl₃, 400 MHz): 1.49 (6H, d), 2.16-2.19 (2H, m), 2.48 (2H, t), 3.08 (2H, t), 4.80-4.84 (1H, m), 7.15 (1H, d), 7.95 (1H, d), 7.99 (1H, s), 8.34-8.37 (1H, dd), 8.45 (1H, d), 8.76 (1H, d). MS (ES): C₂₁H₂₀N₄O₄ requires 392; found 393.2 (M+H⁺).

### EXAMPLE-22

### 3-[5-(5-{3-cyano-4-[(1-methylethyl)oxy]phenyl}-1,2,4-oxadiazol-3-yl)-2-fluorophenyl]propanoic acid (E22)

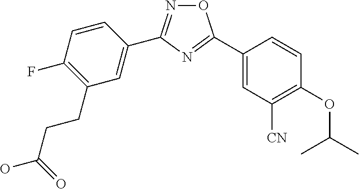

Sodium hydroxide (79 mg) was added to a solution of ethyl 3-[5-(5-{3-cyano-4-[(1-methylethyl)oxy]phenyl}-1,2,4-oxadiazol-3-yl)-2-fluorophenyl]propanoate (168 mg) in ⁱPrOH (3 ml) and water (3 ml). The resulting mixture was stirred at 50° C. for 1 hour. Then 2 M HCl solution was added until pH was about 6. The solvent was evaporated, and the residue was extracted by EtOAc (5 ml, three times). The precipitate was purified by Mass Directed Auto Prep to afford 3-[5-(5-{3-cyano-4-[(1-methylethyl)oxy]phenyl}-1,2,4-oxadiazol-3-yl)-2-fluorophenyl]propanoic acid (31 mg) as a white solid. δH (CDCl₃, 400 MHz): 1.41 (6H, d), 2.70 (2H, t), 3.01 (2H, t), 4.71-4.74 (1H, m), 7.04-7.12 (2H, m), 7.94-8.00 (1H, m), 8.26 (1H, d), 8.35 (1H, s). MS (ES): C₂₀H₁₈FN₃O₄ requires 395; found 396.2 (M+H⁺).

### EXAMPLE-23

### 4-[5-(5-{3-cyano-4-[(1-methylethyl)oxy]phenyl}-1,2,4-oxadiazol-3-yl)-2-fluorophenyl]butanoic acid (E23)

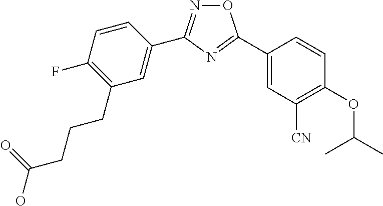

Sodium hydroxide (77 mg) was added to a solution of ethyl 4-[5-(5-{3-cyano-4-[(1-methylethyl)oxy]phenyl}-1,2,4-oxadiazol-3-yl)-2-fluorophenyl]butanoate (168 mg) in ⁱPrOH (3 ml) and water (3 ml). The resulting mixture was stirred at 60° C. for 2.5 hours. Then 2 M HCl solution was added until pH was about 6. The solvent was evaporated, and the residue was extracted by EtOAc (5 ml, three times). The precipitate was purified by Mass Directed Auto Prep to afford 4-[5-(5-{3-cyano-4-[(1-methylethyl)oxy]phenyl}-1,2,4-oxadiazol-3-yl)-2-fluorophenyl]butanoic acid (31 mg) as a white solid. δH (CDCl₃, 400 MHz): 1.44 (6H, d), 2.22-2.24 (2H, m), 2.70 (2H, t), 3.01 (2H, t), 4.71-4.74 (1H, m), 7.04-7.12 (2H, m), 7.94-8.00 (1H, m), 8.26 (1H, d), 8.35 (1H, s). MS (ES): C₂₂H₂₀FN₃O₄ requires 409; found 410.2 (M+H⁺).

### EXAMPLE-24

### 3-[3-(5-{3-cyano-4-[(1-methylethyl)oxy]phenyl}-1,2,4-oxadiazol-3-yl)-2-(trifluoromethyl)phenyl]propanoic acid (E24)

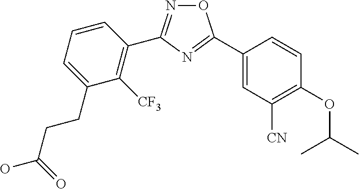

Sodium hydroxide (42 mg) was added to a solution of ethyl 3-[3-(5-{3-cyano-4-[(1-methylethyl)oxy]phenyl}-1,2,4-oxadiazol-3-yl)-2-(trifluoromethyl)phenyl]propanoate (121 mg) in ⁱPrOH (3 ml) and water (3 ml). The resulting mixture was stirred at 50° C. for 1 hour. Then 2 M HCl solution was added until pH was about 6. The solvent was evaporated, and the residue was extracted by EtOAc (5 ml, three times). The precipitate was purified by Mass Directed Auto Prep to afford 3-[3-(5-{3-cyano-4-[(1-methylethyl)oxy]phenyl}-1,2,4-oxadiazol-3-yl)-2-(trifluoromethyl)phenyl]propanoic acid (50 mg) as a white solid. δH (CDCl₃, 400 MHz): 1.40 (6H, d), 2.69 (2H, t), 3.19 (2H, t), 4.71-4.74 (1H, m), 7.05 (1H, d), 7.39-7.41 (1H, m), 7.52-7.54 (2H, m), 8.23-8.26 (1H, dd), 8.33 (1H, d). MS (ES): C₂₂H₁₈F₃N₃O₄ requires 445; found 446.2 (M+H⁺).

### EXAMPLE-25

### 4-[3-(5-{3-cyano-4-[(1-methylethyl)oxy]phenyl}-1,2,4-oxadiazol-3-yl)-2-(trifluoromethyl)phenyl]butanoic acid (E25)

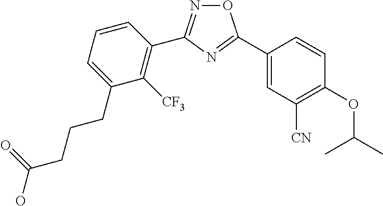

Sodium hydroxide (44 mg) was added to a solution of ethyl 4-[3-(5-{3-cyano-4-[(1-methylethyl)oxy]phenyl}-1,2,4-oxadiazol-3-yl)-2-(trifluoromethyl)phenyl]butanoate (108 mg) in ⁱPrOH (3 ml) and water (3 ml). The resulting mixture was stirred at 50° C. for 1 hour. Then 2 M HCl solution was added until pH was about 6. The solvent was evaporated, and the residue was extracted by EtOAc (5 ml, three times). The precipitate was purified by Mass Directed Auto Prep to afford 4-[3-(5-{3-cyano-4-[(1-methylethyl)oxy]phenyl}-1,2,4-oxadiazol-3-yl)-2-(trifluoromethyl)phenyl]butanoic acid (31 mg) as a white solid. δH (CDCl₃, 400 MHz): δH (CDCl₃, 400 MHz): 1.40 (6H, d), 2.24-2.26 (2H, m), 2.69 (2H, t), 3.19 (2H, t), 4.71-4.74 (1H, m), 7.05 (1H, d), 7.39-7.41 (1H, m), 7.52-7.54 (2H, m), 8.23-8.26 (1H, dd), 8.33 (1H, d). MS (ES): C₂₃H₂₀F₃N₃O₄ requires 459; found 460.2 (M+H⁺).

### EXAMPLE-26

### 3-(5-{3-cyano-4-[(1-methylethyl)oxy]phenyl}-1,2,4-oxadiazol-3-yl)-2-methylbenzoic acid (E26)

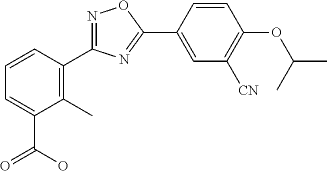

5-[3-(3-formyl-2-methylphenyl)-1,2,4-oxadiazol-5-yl]-2-[(1-methylethyl)oxy]benzonitrile (64 mg) were well dissolved in THF (3 ml). In the resulting solution was added m-CPBA (95 mg), stirred at room temperature for 5.5 hours, LCMS show complete conversion, evaporate off the solvent, EtOAc (10 ml) added, the organic layer was washed water (5 ml) 3 times, evaporate off the solvent, The precipitate was purified by Mass Directed Auto Prep to afford the desired compound 3-(5-{3-cyano-4-[(1-methylethyl)oxy]phenyl}-1,2,4-oxadiazol-3-yl)-2-methylbenzoic acid (29 mg). δH (CDCl₃, 400 MHz): δH (CDCl₃, 400 MHz): 1.48 (6H, d), 2.86 (3H, s), 4.77-4.84 (1H, m), 7.14 (1H, d), 7.45 (1H, dd), 8.07-8.15 (1H, dd), 8.34-8.37 (1H, dd), 8.44 (1H, d). MS (ES): C₂₀H₁₇N₃O₃ requires 363; found 364.2 (M+H⁺).

### EXAMPLE-27

### {[3-(5-{5-chloro-6-[(1-methylethyl)oxy]-3-pyridinyl}-1,2,4-oxadiazol-3-yl)-2-methylphenyl]oxy}acetic acid (E27)

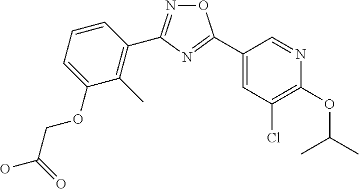

Sodium hydroxide (17 mg) was added to a solution of ethyl {[3-(5-{5-chloro-6-[(1-methylethyl)oxy]-3-pyridinyl}-1,2,4-oxadiazol-3-yl)-2-methylphenyl]oxy}acetate (47 mg) in ⁱPrOH (4 ml) and water (4 ml). The resulting mixture was stirred at 50° C. for 1 hour. Then 2 M HCl solution was added until pH was about 6. The solvent was evaporated, and the residue was extracted by EtOAc (5 ml, three times). The precipitate was purified by Mass Directed Auto Prep to afford {[3-(5-{5-chloro-6-[(1-methylethyl)oxy]-3-pyridinyl}-1,2,4-oxadiazol-3-yl)-2-methylphenyl]oxy}acetic acid (12 mg) as a white solid. δH (CDCl₃, 400 MHz): δH (CDCl₃, 400 MHz): 1.43 (6H, d), 2.54 (3H, s), 4.71 (2H, s), 5.51-5.54 (1H, m), 7.05 (1H, d), 7.28-7.32 (1H, dd), 7.55 (1H, d), 8.48 (1H, d), 8.90 (1H, d). MS (ES): C₁₉H₁₈ClN₃O₅ requires 403; found 404.2 (M+H⁺).

### EXAMPLE-28

### 4-[3-(5-{3-chloro-4-[(1-methylethyl)oxy]phenyl}-1,2,4-oxadiazol-3-yl)-2-ethylphenyl]butanoic acid (E28)

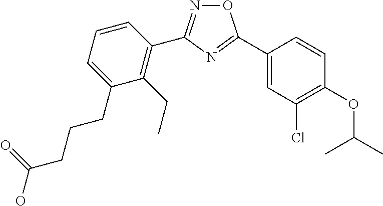

Sodium hydroxide (50 mg) was added to a solution of ethyl 4-[3-(5-{3-chloro-4-[(1-methylethyl)oxy]phenyl}-1,2,4-oxadiazol-3-yl)-2-ethylphenyl]butanoate (D54) (170 mg) in tetrahydrofuran (THF) (3 mL) and water (3 mL). The resulting mixture was stirred and heated at 90° C. for 6 h. After cooling the reaction, the mixture was neutralized with 0.5 M HCl solution until pH was about 6. The solvent was evaporated, and the residue was extracted with EtOAc (5 mL) for three times. The organic phases were evaporated in vacuo and the residue was purified by Mass Directed Auto Prep to afford 4-[3-(5-{3-chloro-4-[(1-methylethyl)oxy]phenyl}-1,2,4-oxadiazol-3-yl)-2-ethylphenyl]butanoic acid (E28) (84 mg) as a white solid. δH (DMSO-d₆, 400 MHz): 1.12 (3H, t), 1.36 (6H, d), 1.80 (2H, m), 2.33 (2H, t), 2.72 (2H, m), 2.93 (2H, m), 4.89 (1H, s), 7.33 (1H, t), 7.42 (2H, m), 7.66 (1H, dd), 8.10 (1H, dd), 8.17 (1H, d), 12.12 (1H, br s). MS (ES): C₂₃H₂₆ClN₂O₄ requires 428; found 429.2 (M+H⁺).

### EXAMPLE-29

### 3-[3-(5-{3-chloro-4-[(1-methylethyl)oxy]phenyl}-1,2,4-oxadiazol-3-yl)-2-ethylphenyl]propanoic acid (E29)

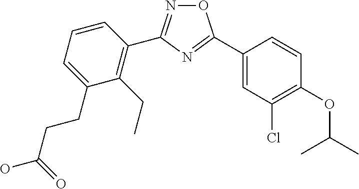

Sodium hydroxide (50 mg) was added to a solution of ethyl 3-[3-(5-{3-chloro-4-[(1-methylethyl)oxy]phenyl}-1,2,4-oxadiazol-3-yl)-2-ethylphenyl]propanoate (D55) (164 mg) in tetrahydrofuran (THF) (3 mL) and water (3 mL). The resulting mixture was stirred and heated at 90° C. for 4 h. After cooling the reaction, the mixture was neutralized with 0.5 M HCl solution until pH was about 6. The solvent was evaporated, and the residue was extracted with EtOAc (5 mL) for three times. The organic phases were evaporated in vacuo and the residue was purified by Mass Directed Auto Prep to afford 3-[3-(5-{3-chloro-4-[(1-methylethyl)oxy]phenyl}-1,2,4-oxadiazol-3-yl)-2-ethylphenyl]propanoic acid (E29) (83 mg) as a white solid. δH (DMSO-d₆, 400 MHz): 1.14 (3H, t), 1.36 (6H, d), 2.57 (2H, t), 2.94 (4H, m), 4.89 (1H, m), 7.32 (1H, t), 7.44 (2H, m), 7.67 (1H, dd), 8.10 (1H, dd), 8.17 (1H, d), 12.23 (1H, s). MS (ES): C₂₂H₂₃ClN₂O₄ requires 414; found 415.2 (M+H⁺).

### EXAMPLE-30

### 3-[3-(5-{3-cyano-4-[(1-methylethyl)oxy]phenyl}-1,2,4-oxadiazol-3-yl)-5-fluoro-2-(methyloxy)phenyl]propanoic acid (E30)

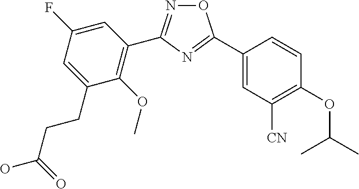

Sodium hydroxide (50 mg) was added to a solution of ethyl 3-[3-(5-{3-cyano-4-[(1-methylethyl)oxy]phenyl}-1,2,4-oxadiazol-3-yl)-5-fluoro-2-(methyloxy)phenyl]propanoate (D59) (128 mg) in tetrahydrofuran (THF) (2 mL) and water (2 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 3-[3-(5-{3-cyano-4-[(1-methylethyl)oxy]phenyl}-1,2,4-oxadiazol-3-yl)-5-fluoro-2-(methyloxy)phenyl]propanoic acid (E30) (74 mg) as a yellow solid. δH (DMSO-d₆, 400 MHz): 1.38 (6H, d), 2.62 (2H, t), 2.94 (2H, t), 3.76 (3H, s), 4.99 (1H, m), 7.42 (1H, dd), 7.56 (1H, d), 7.64 (1H, dd), 8.42 (1H, dd), 8.54 (1H, d), 12.25 (1H, br s). δF (DMSO-d₆, 376 MHz): −117.8. MS (ES): C₂₂H₂₀FN₃O₅ requires 425; found 426.2 (M+H⁺).

### EXAMPLE-31

### 4-[3-(5-{3-cyano-4-[(1-methylethyl)oxy]phenyl}-1,2,4-oxadiazol-3-yl)-5-fluoro-2-(methyloxy)phenyl]butanoic acid (E31)

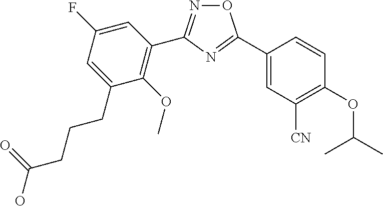

Sodium hydroxide (50 mg) was added to a solution of ethyl 4-[3-(5-{3-cyano-4-[(1-methylethyl)oxy]phenyl}-1,2,4-oxadiazol-3-yl)-5-fluoro-2-(methyloxy)phenyl]butanoate (D60) (100 mg) in tetrahydrofuran (THF) (3 mL) and water (3 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 4-[3-(5-{3-cyano-4-[(1-methylethyl)oxy]phenyl}-1,2,4-oxadiazol-3-yl)-5-fluoro-2-(methyloxy)phenyl]butanoic acid (E31) (65 mg) as a white solid. δH (DMSO-d₆, 400 MHz): 1.38 (6H, d), 1.85 (2H, m), 2.29 (2H, t), 2.72 (2H, m), 3.75 (3H, s), 4.99 (1H, m), 7.39 (1H, dd), 7.56 (1H, d), 7.64 (1H, dd), 8.42 (1H, dd), 8.54 (1H, d), 12.10 (1H, s). δF (DMSO-d₆, 376 MHz): −117.8. MS (ES): C₂₃H₂₂FN₃O₅ requires 439; found 440.2 (M+H⁺).

### EXAMPLE-32

### 4-[3-(5-{5-chloro-6-[(1-methylethyl)oxy]-3-pyridinyl}-1,2,4-oxadiazol-3-yl)-5-fluoro-2-(methyloxy)phenyl]butanoic acid (E32)

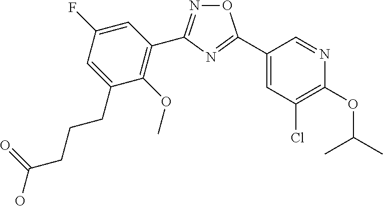

Sodium hydroxide (50 mg) was added to a solution of ethyl 4-[3-(5-{5-chloro-6-[(1-methylethyl)oxy]-3-pyridinyl}-1,2,4-oxadiazol-3-yl)-5-fluoro-2-(methyloxy)phenyl]butanoate (D62) (120 mg) in tetrahydrofuran (THF) (3 mL) and water (3 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 4-[3-(5-{5-chloro-6-[(1-methylethyl)oxy]-3-pyridinyl}-1,2,4-oxadiazol-3-yl)-5-fluoro-2-(methyloxy)phenyl]butanoic acid (E32) (60 mg) as a white solid. δH (DMSO-d₆, 400 MHz): 1.39 (6H, d), 1.85 (2H, m), 2.29 (2H, t), 2.71 (2H, t), 3.75 (3H, s), 5.46 (1H, m), 7.39 (1H, dd), 7.64 (1H, dd), 8.58 (1H, d), 8.95 (1H, d), 12.11 (1H, s). δF (DMSO-d₆, 376 MHz): −117.8. MS (ES): C₂₁H₂₁ClFN₃O₆ requires 449; found 450.1 (M+H⁺).

### EXAMPLE-33

### 3-[3-(5-{5-chloro-6-[(1-methylethyl)oxy]-3-pyridinyl}-1,2,4-oxadiazol-3-yl)-5-fluoro-2-(methyloxy)phenyl]propanoic acid (E33)

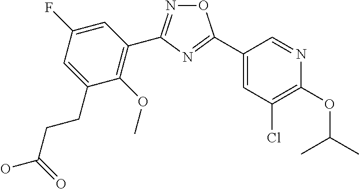

Sodium hydroxide (50 mg) was added to a solution of ethyl 3-[3-(5-{5-chloro-6-[(1-methylethyl)oxy]-3-pyridinyl}-1,2,4-oxadiazol-3-yl)-5-fluoro-2-(methyloxy)phenyl]propanoate (D63) (140 mg) in tetrahydrofuran (THF) (3 mL) and water (3 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 3-[3-(5-{5-chloro-6-[(1-methylethyl)oxy]-3-pyridinyl}-1,2,4-oxadiazol-3-yl)-5-fluoro-2-(methyloxy)phenyl]propanoic acid (E33) (84 mg) as a white solid. δH (DMSO-d₆, 400 MHz): 1.39 (6H, d), 2.62 (2H, t), 2.94 (2H, t), 3.76 (3H, s), 5.46 (1H, m), 7.42 (1H, dd), 7.65 (1H, dd), 8.58 (1H, d), 8.95 (1H, d), 12.25 (1H, s). SF (DMSO-d₆, 376 MHz): −117.8. MS (ES): C₂₀H₁₉ClFN₃O₆ requires 435; found 436.1 (M+H⁺).

### EXAMPLE-34

### 3-[3-(5-{3-chloro-4-[(1-methylethyl)oxy]phenyl}-1,2,4-oxadiazol-3-yl)-5-fluoro-2-(methyloxy)phenyl]propanoic acid (E34)

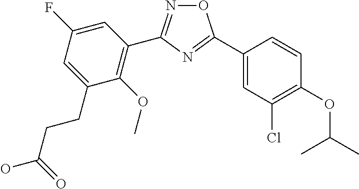

Sodium hydroxide (50 mg) was added to a solution of ethyl 3-[3-(5-{3-chloro-4-[(1-methylethyl)oxy]phenyl}-1,2,4-oxadiazol-3-yl)-5-fluoro-2-(methyloxy)phenyl]propanoate (D65) (109 mg) in tetrahydrofuran (THF) (3 mL) and water (3 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 3-[3-(5-{3-chloro-4-[(1-methylethyl)oxy]phenyl}-1,2,4-oxadiazol-3-yl)-5-fluoro-2-(methyloxy)phenyl]propanoic acid (E34) (58 mg) as a white solid. δH (DMSO-d₆, 400 MHz): 1.36 (6H, d), 2.62 (2H, t), 2.93 (2H, t), 3.75 (3H, s), 4.89 (1H, m), 7.42 (2H, m), 7.64 (1H, dd), 8.12 (1H, dd), 8.20 (1H, d), 12.25 (1H, s). δF (DMSO-d₆, 376 MHz): −117.8. MS (ES): C₂₁H₂₀ClFN₂O₆ requires 434; found 435.1 (M+H⁺).

### EXAMPLE-35

### 4-[3-(5-{3-chloro-4-[(1-methylethyl)oxy]phenyl}-1,2,4-oxadiazol-3-yl)-5-fluoro-2-(methyloxy)phenyl]butanoic acid (E35)

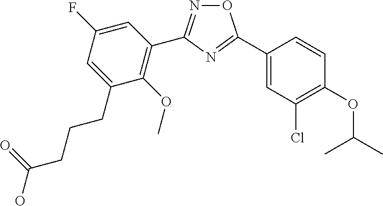

Sodium hydroxide (50 mg) was added to a solution of ethyl 4-[3-(5-{3-chloro-4-[(1-methylethyl)oxy]phenyl}-1,2,4-oxadiazol-3-yl)-5-fluoro-2-(methyloxy)phenyl]butanoate (D66) (95 mg) in tetrahydrofuran (THF) (3 mL) and water (3 mL). The resulting mixture was stirred and heated at 90° C. for 2 h. After cooling the reaction, the mixture was neutralized with 1 M HCl solution until pH was about 6. The solvent was evaporated, and the residue was extracted with EtOAc (5 mL) for three times. The organic phases were evaporated in vacuo and the residue was purified by Mass Directed Auto Prep to afford 4-[3-(5-{3-chloro-4-[(1-methylethyl)oxy]phenyl}-1,2,4-oxadiazol-3-yl)-5-fluoro-2-(methyloxy)phenyl]butanoic acid (E35) (20 mg) as a white solid. δH (DMSO-d₆, 400 MHz): 1.29 (6H, d), 1.78 (2H, m), 2.22 (2H, t), 2.65 (2H, t), 3.67 (3H, s), 4.82 (1H, m), 7.30 (1H, dd), 7.37 (1H, d), 7.55 (1H, dd), 8.06 (1H, dd), 8.13 (1H, d), 12.03 (1H, s). δF (DMSO-d₆, 376 MHz): −117.8. MS (ES): C₂₂H₂₂ClFN₂O₆ requires 448; found 449.2 (M+H⁺).

### EXAMPLE-36

### 4-[3-(5-{3-chloro-4-[(1-methylethyl)oxy]phenyl}-1,2,4-oxadiazol-3-yl)-2-propylphenyl]butanoic acid (E36)

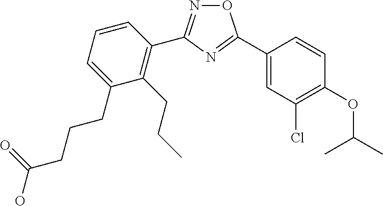

Sodium hydroxide (50 mg) was added to a solution of ethyl 4-[3-(5-{3-chloro-4-[(1-methylethyl)oxy]phenyl}-1,2,4-oxadiazol-3-yl)-2-propylphenyl]butanoate (D69) (85 mg) in tetrahydrofuran (THF) (3 mL) and water (3 mL). The resulting mixture was stirred at room temperature for 2 h. The mixture was neutralized with 1 M HCl solution until pH was about 6. The solvent was evaporated, and the residue was dissolved in THF, and filtered. The filtrate was purified by Mass Directed Auto Prep to afford 4-[3-(5-{3-chloro-4-[(1-methylethyl)oxy]phenyl}-1,2,4-oxadiazol-3-yl)-2-propylphenyl]butanoic acid (E36)

(36 mg) as a white solid. δH (DMSO-d₆, 400 MHz): 0.93 (3H, t), 1.36 (61-1, d), 1.50 (2H, m), 1.79 (2H, m), 2.33 (2H, t), 2.71 (2H, t), 2.88 (2H, t), 4.89 (1H, m), 7.32 (1H, t), 7.40 (1H, d), 7.45 (1H, d), 7.68 (1H, dd), 8.10 (1H, dd), 8.18 (1H, d), 12.13 (1H, s). MS (ES): C₂₄H₂₇ClN₂O₄ requires 442; found 443.2 (M+H⁺).

### EXAMPLE-37

### 4-[3-(5-{3-cyano-4-[(1-methylethyl)oxy]phenyl}-1,2,4-oxadiazol-3-yl)-2-propylphenyl]butanoic acid (E37)

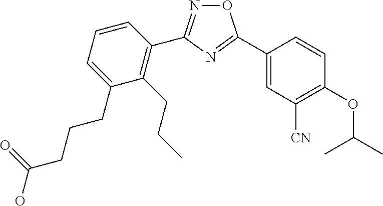

Sodium hydroxide (50 mg) was added to a solution of ethyl 4-[3-(5-{3-cyano-4-[(1-methylethyl)oxy]phenyl}-1,2,4-oxadiazol-3-yl)-2-propylphenyl]butanoate (D70) (136 mg) in tetrahydrofuran (THF) (3 mL) and water (3 mL). The resulting mixture was stirred at room temperature for 2 h. The mixture was neutralized with 1 M HCl solution until pH was about 6. The solvent was evaporated, and the residue was dissolved in THF, and filtered. The filtrate was purified by Mass Directed Auto Prep to afford 4-[3-(5-{3-cyano-4-[(1-methylethyl)oxy]phenyl}-1,2,4-oxadiazol-3-yl)-2-propylphenyl]butanoic acid (E37) (32 mg) as a white solid. δH (DMSO-d₆, 400 MHz): 0.93 (3H, t), 1.38 (8H, m), 1.80 (2H, m), 2.33 (2H, t), 2.70 (2H, t), 2.88 (2H, m), 4.97 (1H, m), 7.31 (1H, t), 7.39 (1H, d), 7.54 (1H, d), 7.68 (1H, d), 8.38 (1H, d), 8.47 (1H, s), 12.14 (1H, br s). MS (ES): C₂₅H₂₇N₃O₄ requires 433; found 434.3 (M+H⁺).

### EXAMPLE-38

### 4-{[3-(5-{3-cyano-4-[(1-methylethyl)oxy]phenyl}-1,2,4-oxadiazol-3-yl)-2-ethylphenyl]oxy}butanoic acid (E38)

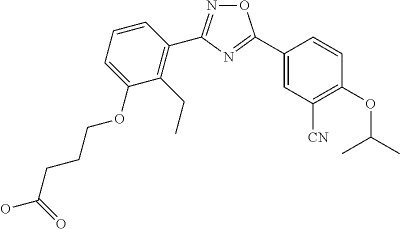

Sodium hydroxide (63.4 mg) was added to a solution of ethyl 4-{[3-(5-{3-cyano-4-[(1-methylethyl)oxy]phenyl}-1,2,4-oxadiazol-3-yl)-2-ethylphenyl]oxy}butanoate (D77) (73.5 mg) in tetrahydrofuran (THF) (10 mL), ethanol (3 mL) and water (1 mL). The resulting mixture was stirred at room temperature overnight. The mixture was neutralized with 2 M HCl solution until pH was about 6. The solvent was evaporated, and the residue was dissolved in THF, and filtered. The filtrate was purified by Mass Directed Auto Prep to afford 4-{[3-(5-{3-cyano-4-[(1-methylethyl)oxy]phenyl}-1,2,4-oxadiazol-3-yl)-2-ethylphenyl]oxy}butanoic acid (E38) (19.6 mg). δH (DMSO-d₆, 400 MHz): 1.15 (3H, t), 1.38 (6H, d), 2.01 (2H, m), 2.45 (2H, t), 2.89 (2H, m), 4.07 (2H, t), 4.98 (1H, m), 7.19 (1H, d), 7.35 (1H, t), 7.44 (1H, d), 7.56 (1H, d), 8.39 (1H, dd), 8.49 (1H, d), 12.18 (1H, br s). MS (ES): C₂₄H₂₅N₃O₅ requires 435; found 436.2 (M+H⁺).

### EXAMPLE-39

### 4-{[3-(5-{3-chloro-4-[(1-methylethyl)oxy]phenyl}-1,2,4-oxadiazol-3-yl)-2-ethylphenyl]oxy}butanoic acid (E39)

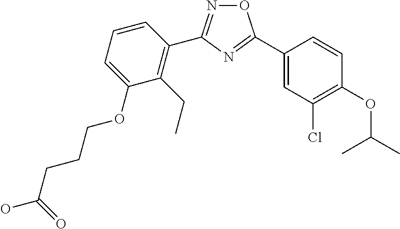

Sodium hydroxide (71.5 mg) was added to a solution of ethyl 4-{[3-(5-{3-chloro-4-[(1-methylethyl)oxy]phenyl}-1,2,4-oxadiazol-3-yl)-2-ethylphenyl]oxy}butanoate (D78) (84.5 mg) in tetrahydrofuran (THF) (10 mL), ethanol (3 mL) and water (1 mL). The resulting mixture was stirred at room temperature overnight. The mixture was neutralized with 2 M HCl solution until pH was about 6. The solvent was evaporated, and the residue was dissolved in THF, and filtered. The filtrate was purified by Mass Directed Auto Prep to afford 4-{[3-(5-{3-chloro-4-[(1-methylethyl)oxy]phenyl}-1,2,4-oxadiazol-3-yl)-2-ethylphenyl]oxy}butanoic acid (E39) (21 mg). δH (DMSO-d₆, 400 MHz): 1.14 (3H, t), 1.36 (6H, d), 2.02 (2H, m), 2.45 (2H, t), 2.90 (2H, m), 4.07 (2H, t), 4.88 (1H, m), 7.18 (1H, d), 7.34 (1H, t), 7.44 (2H, m), 8.09 (1H, dd), 8.16 (1H, d), 12.17 (1H, br s). MS (ES): C₂₃H₂₆ClN₂O₆ requires 444; found 445.2 (M+H⁺).

### EXAMPLE-40

### {[3-(5-{3-cyano-4-[(1-methylethyl)oxy]phenyl}-1,2,4-oxadiazol-3-yl)-2-ethylphenyl]oxy}acetic acid (E40)

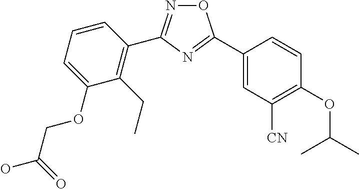

Sodium hydroxide (50 mg) was added to a solution of ethyl {[3-(5-{3-cyano-4-[(1-methylethyl)oxy]phenyl}-1,2,4-oxadiazol-3-yl)-2-ethylphenyl]oxy}acetate (D85) (35 mg) in isopropanol (3 mL) and water (1 mL). The resulting mixture was stirred at room temperature for 2 h. The mixture was neutralized with 1 M HCl solution until pH was about 6. The solvent was evaporated, and the residue was dissolved in THF, and filtered. The filtrate was purified by Mass Directed Auto Prep to afford {[3-(5-{3-cyano-4-[(1-methylethyl)oxy]phenyl}-1,2,4-oxadiazol-3-yl)-2-ethylphenyl]oxy}acetic acid (E40) (24 mg). δH (DMSO-d₆, 400 MHz): 1.16 (3H, t), 1.38 (6H, d), 2.94 (2H, m), 4.81 (2H, s), 4.98 (1H, m), 7.12 (1H, d), 7.34 (1H, t), 7.45 (1H, d), 7.56 (1H, d), 8.40 (1H, dd), 8.50 (1H, d), 13.05 (1H, s). MS (ES): C₂₂H₂₁N₃O₆ requires 407; found 408.2 (M+H⁺).

### EXAMPLE-41

### 4-[3-(5-{3-cyano-4-[(1-methylethyl)oxy]phenyl}-1,3,4-oxadiazol-2-yl)-2-ethylphenyl]butanoic acid (E41)

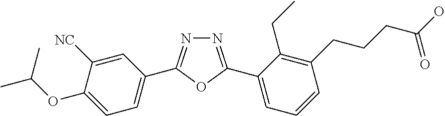

To a solution of ethyl 4-[3-(5-{3-cyano-4-[(1-methylethyl)oxy]phenyl}-1,3,4-oxadiazol-2-yl)-2-ethylphenyl]butanoate (D88) (40 mg) in THF (5 mL) and water (0.5 mL) was added 2 M Sodium hydroxide (0.045 mL). The resulting mixture was stirred at room temperature overnight. The mixture was neutralized with 1 M HCl solution until pH was about 6. The solvent was evaporated, and the residue was dissolved in THF, and filtered. The filtrate was purified by Mass Directed Auto Prep to afford 4-[3-(5-{3-cyano-4-[(1-methylethyl)oxy]phenyl}-1,3,4-oxadiazol-2-yl)-2-ethylphenyl]butanoic acid (E41) (22 mg). δH (DMSO-d₆, 400 MHz): 1.17 (3H, t), 1.38 (6H, d), 1.80 (2H, m), 2.32 (2H, t), 2.73 (2H, t), 3.08 (2H, m), 4.95 (1H, m), 7.37 (1H, m), 7.45 (1H, d), 7.54 (1H, d), 7.89 (1H, dd), 8.32 (1H, dd), 8.44 (1H, d). MS (ES): C₂₄H₂₅N₃O₄ requires 419; found 420.2 (M+H⁺).

### EXAMPLE-42

### 4-[3-(5-{3-cyano-4-[(1-methylethyl)oxy]phenyl}-1,3,4-thiadiazol-2-yl)-2-ethylphenyl]butanoic acid (E42)

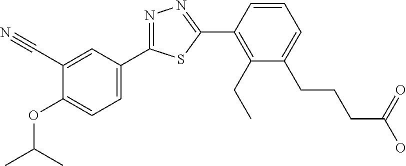

To a solution of ethyl 4-[3-(5-{3-cyano-4-[(1-methylethyl)oxy]phenyl}-1,3,4-thiadiazol-2-yl)-2-ethylphenyl]butanoate (D97) (57 mg) in tetrahydrofuran (THF) (3 mL) and water (3 mL) was added sodium hydroxide (50 mg). The reaction mixture was heated at 90° C. for 4 h and stirred at room temperature for 48 h. After cooling the reaction, the mixture was neutralized with 1 M HCl solution. The solvent was removed in vacuo. The residue was dissolved in THF, and filtered. The filtrate was purified by Mass Directed AutoPrep to give 4-[3-(5-{3-cyano-4-[(1-methylethyl)oxy]phenyl}-1,3,4-thiadiazol-2-yl)-2-ethylphenyl]butanoic acid (E42) (18 mg) as a pale yellow solid. δH (DMSO-d₆, 400 MHz): 1.09 (3H, t), 1.36 (6H, d), 1.80 (2H, m), 2.35 (2H, t), 2.71 (2H, m), 2.84 (2H, q), 4.93 (1H, m), 7.31 (1H, m), 7.40 (2H, m), 7.48 (1H, d), 8.28 (1H, m), 8.38 (1H, d). MS (ES): C₂₄H₂₅N₃O₃S requires 435; found 436.2 (M+H⁺).

### EXAMPLE-43

### N-{[3-(5-{3-chloro-4-[(1-methylethyl)oxy]phenyl}-1,3,4-thiadiazol-2-yl)-2-ethylphenyl]methyl}-N-methylglycine (E43)

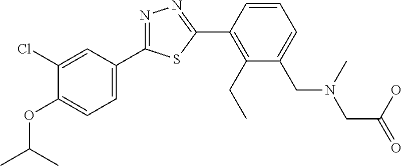

To a mixture of 3-(5-{3-chloro-4-[(1-methylethyl)oxy]phenyl}-1,3,4-thiadiazol-2-yl)-2-ethylbenzaldehyde (D101) (100 mg) and N-methylglycine (461 mg) in dichloromethane (DCM) (5 mL) was added one drop of acetic acid. The reaction mixture was sitrred at room temperature for 10 min. After that, sodium triacetoxyborohydride (164 mg) was added to the reaction mixture. The reaction mixture was stirred at room temperature overnight. Then water was added to the reaction mixture. Then the resulting solution extracted with EA. The combined organic solution was washed with water and dried over anhydrous sodium sulphate. After filtration and concentration, the residue was purified by Mass Directed AutoPrep to give N-{[3-(5-{3-chloro-4-[(1-methylethyl)oxy]phenyl}-1,3,4-thiadiazol-2-yl)-2-ethylphenyl]methyl}-N-methylglycine (E43) (28.4 mg). δH (DMSO-d₆, 400 MHz): 1.08 (3H, t), 1.35 (6H, d), 2.66 (3H, s), 2.98 (2H, m), 3.95 (2H, s), 4.30 (2H, s), 4.84 (1H, m), 7.38 (1H, d), 7.47 (1H, m), 7.67 (2H, m), 7.97 (1H, d), 8.10 (1H, d). δF (DMSO-d₆, 376 MHz): −73.6. MS (ES): C₂₃H₂₆ClN₃O₃S requires 459; found 460.2 (M+H⁺).

### EXAMPLE-44

### 4-[3-(3-{3-cyano-4-[(1-methylethyl)oxy]phenyl}-1,2,4-thiadiazol-5-yl)-2-ethylphenyl]butanoic acid (E44)

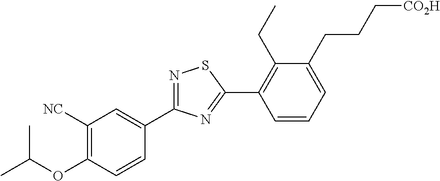

To a solution of ethyl 4-[3-(3-{3-cyano-4-[(1-methylethyl)oxy]phenyl}-1,2,4-thiadiazol-5-yl)-2-ethylphenyl]butanoate (D104) (60 mg) in isopropanol (3.00 mL) and water (3 mL) was added sodium hydroxide (50 mg). The reaction mixture was stirred at room temperature for 2 h. The reaction mixture was neutralized with 1 M HCl solution. The solvent was removed in vacuo. The residue was dissolved in THF and filtered. The filtrate was purified by Mass Directed AutoPrep to give 4-[3-(3-{3-cyano-4-[(1-methylethyl)oxy]phenyl}-1,2,4-thiadiazol-5-yl)-2-ethylphenyl]butanoic acid (E44) (2 mg) as a yellow gel. δH (DMSO-d₅, 400 MHz): 1.19 (3H, m), 1.37 (6H, d), 1.81 (2H, m), 2.34 (2H, t), 2.74 (2H, t), 2.96 (2H, d), 4.93 (1H, m), 7.35 (1H, t), 7.45 (1H, d), 7.50 (1H, d), 7.64 (1H, d), 8.50 (2H, m), 12.13 (1H, br s). δF (DMSO-d₆, 376 MHz): −73.5. MS (ES): C₂₄H₂₅N₃O₃S requires 435; found 436.2 (M+H⁺).

### EXAMPLE-45

### 4-[3-(5-{3-chloro-4-[(1-methylethyl)oxy]phenyl}-1,2,4-thiadiazol-3-yl)-2-ethylphenyl]butanoic acid (E45)

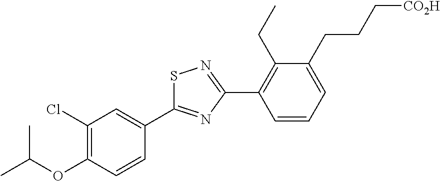

To a mixture of ethyl 4-[3-(5-{3-chloro-4-[(1-methylethyl)oxy]phenyl}-1,2,4-thiadiazol-3-yl)-2-ethylphenyl]butanoate (D106) (51 mg) in isopropanol (40 mL) and water (5 mL) at room temperature was added NaOH (8.62 mg). The mixture was The reaction vessel was sealed and heated under microwave at 80° C. for 2 h. After cooling the reaction, the mixture was neutralized with 1 M HCl solution. The solvent was removed in vacuo. The residue was dissolved in THF and filtered. The filtrate was purified by Mass Directed AutoPrep to give 4-[3-(5-{3-chloro-4-[(1-methylethyl)oxy]phenyl}-1,2,4-thiadiazol-3-yl)-2-ethylphenyl]butanoic acid (E45) (31 mg) as a brown solid. δH (DMSO-d₆, 400 MHz): 1.10 (3H, t), 1.35 (6H, m), 1.81 (2H, m), 2.33 (2H, t), 2.72 (2H, t), 2.92 (2H, m), 4.86 (1H, m), 7.28 (1H, t), 7.33 (1H, d), 7.39 (1H, d), 7.65 (1H, dd), 8.02 (1H, dd), 8.14 (1H, d), 12.12 (1H, s). MS (ES): C₂₃H₂₅ClN₂O₃S requires 444; found 445.2 (M+H⁺).

### EXAMPLE-46

### 4-[3-(5-{3-cyano-4-[(1-methylethyl)oxy]phenyl}-1,2,4-thiadiazol-3-yl)-2-ethylphenyl]butanoic acid (E46)

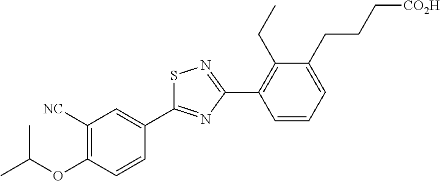

To a solution of ethyl 4-[3-(5-{3-cyano-4-[(1-methylethyl)oxy]phenyl}-1,2,4-thiadiazol-3-yl)-2-ethylphenyl]butanoate (D108) (98 mg) in isopropanol (3.00 mL) and water (3 mL) was added sodium hydroxide (50 mg). The reaction mixture was stirred at room temperature for 2 h. The reaction mixture was neutralized with 1 M HCl solution. The solvent was removed in vacuo. The residue was dissolved in THF, and filtered. The filtrate was purified by Mass Directed AutoPrep to give 4-[3-(5-{3-cyano-4-[(1-methylethyl)oxy]phenyl}-1,2,4-thiadiazol-3-yl)-2-ethylphenyl]butanoic acid (E46) (50 mg) as an off-white solid. δH (DMSO-d₆, 400 MHz): 1.09 (3H, t), 1.37 (6H, d), 1.80 (2H, m), 2.33 (2H, t), 2.71 (2H, m), 2.91 (2H, d), 4.95 (1H, m), 7.28 (1H, t), 7.35 (1H, d), 7.50 (1H, d), 7.65 (1H, dd), 8.34 (1H, dd), 8.48 (1H, d), 12.11 (1H, s). MS (ES): C₂₄H₂₅N₃O₃S requires 435; found 436.2 (M+H⁺).

### EXAMPLE-47

### N-{[3-(5-{3-cyano-4-[(1-methylethyl)oxy]phenyl}-1,2,4-thiadiazol-3-yl)-2-ethylphenyl]methyl}-N-methylglycine (E47)

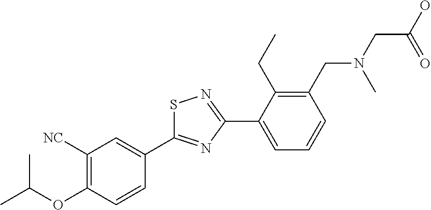

To a solution of 5-[3-(2-ethyl-3-formylphenyl)-1,2,4-thiadiazol-5-yl]-2-[(1-methylethyl)oxy]benzonitrile (D109) (80 mg) in dichloromethane (DCM) (5 mL) and methanol (5.00 mL), was added N-methylglycine (94 mg) and acetic acid (0.036 mL). The resulting solution was stirred at ambient temperature for overnight. Sodium triacetoxyborohydride (135 mg) was added and the reaction solution was stirred for another 2 h. The solvent was removed in vacuo and the residue was purified by Mass Directed AutoPrep to give N-{[3-(5-{3-cyano-4-[(1-methylethyl)oxy]phenyl}-1,2,4-thiadiazol-3-yl)-2-ethylphenyl]methyl}-N-methylglycine (E47) (22 mg) as a white solid. δH (DMSO-d₆, 400 MHz): 1.02 (3H, t), 1.31 (6H, d), 2.24 (3H, s), 2.96 (2H, m), 3.15 (2H, s), 3.70 (2H, s), 4.89 (1H, m), 7.25 (1H, t), 7.44 (2H, m), 7.67 (1H, dd), 8.27 (1H, dd), 8.42 (1H, d). MS (ES): C₂₄H₂₆N₄O₃S requires 450; found 451.2 (M+H⁺).

### EXAMPLE-48

### N-{[3-(5-{3-cyano-4-[(1-methylethyl)oxy]phenyl}-1,2,4-thiadiazol-3-yl)-2-ethylphenyl]methyl}-b-alanine (E48)

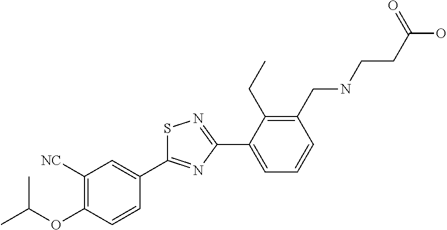

To a solution of 5-[3-(2-ethyl-3-formylphenyl)-1,2,4-thiadiazol-5-yl]-2-[(1-methylethyl)oxy]benzonitrile (D109) (110 mg) in ethanol (5.00 mL) and dichloromethane (DCM) (5 mL) was added hydrogen chloride—ethyl β-alaninate (1:1) (81 mg) and sodium acetate (43.0 mg). The reaction solution was stirred at room temperature for overnight. Sodium triacetoxyborohydride (185 mg) was added and the reaction solution was stirred for another 2 h. The solvent was removed in vacuo and the residue was dissoved in ethyl acetate (50 mL) and washed with water (2*10 mL). After concentration, the residue was dissolved in isopropanol/water (1:1) 10 mL, heated to 90° C. for 2 h. Isopropanol was removed in vacuo and the residue was acidified to pH=6-7, extracted with ethyl acetate (2*15 mL). The combined organic phases were concentrated and the residue was purified by Mass Directed AutoPrep to give N-{[3-(5-{3-cyano-4-[(1-methylethyl)oxy]phenyl}-1,2,4-thiadiazol-3-yl)-2-ethylphenyl]methyl}-β-alanine (E48) (6 mg) as a white solid. δH (DMSO-d₆, 400 MHz): 1.05 (3H, t), 1.31 (6H, d), 2.31 (2H, t), 2.79 (2H, t), 2.90 (2H, m), 3.82 (2H, s), 4.89 (1H, m), 7.27 (1H, t), 7.45 (2H, m), 7.68 (1H, d), 8.27 (1H, dd), 8.42 (1H, d). MS (ES): C₂₄H₂₆N₄O₃S requires 450; found 451.2 (M+H⁺).

### EXAMPLE-49

### N-{2-[3-(5-{3-chloro-4-[(1-methylethyl)oxy]phenyl}-1,2,4-thiadiazol-3-yl)-2-ethylphenyl]ethyl}-N-methylglycine (E49)

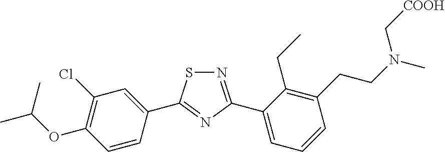

To a solution of [3-(5-{3-chloro-4-[(1-methylethyl)oxy]phenyl}-1,2,4-thiadiazol-3-yl)-2-ethylphenyl]acetaldehyde (D111) (100 mg), N-methylglycine (44.4 mg) and acetic acid (1.428 μL) in dichloromethane (DCM) (8 mL) and N,N-dimethylformamide (DMF) (0.400 mL) stirred under nitrogen at room temperature for 20 min was added sodium triacetoxyborohydride (106 mg). The reaction mixture was stirred at room temperature for 5 h. Water was added to quench the reaction, the mixture was neutralized with acetic acid, the resulting solution was concentrated and the residue was purified by Mass Directed AutoPrep to afford N-{2-[3-(5-{3-chloro-4-[(1-methylethyl)oxy]phenyl}-1,2,4-thiadiazol-3-yl)-2-ethylphenyl]ethyl}-N-methylglycine (E49) (23 mg) as a white solid. δH (DMSO-d₆, 400 MHz): 1.13 (3H, t), 1.35 (6H, d), 2.93 (5H, m), 3.14 (2H, m), 3.32 (2H, m), 4.18 (2H, s), 4.86 (1H, m), 7.34 (1H, t), 7.40 (2H, m), 7.72 (1H, d), 8.02 (1H, dd), 8.14 (1H, d). δF (DMSO-d₆, 376 MHz): −73.5. MS (ES): C₂₄H₂₈ClN₃O₃S requires 473; found 474.1 (M+H⁺).

### EXAMPLE-50

### 4-[3-(2-{3-cyano-4-[(1-methylethyl)oxy]phenyl}-1,3-thiazol-5-yl)-2-ethylphenyl]butanoic acid (E50)

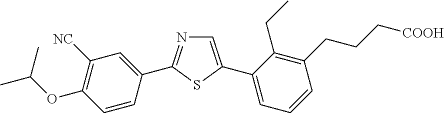

To a solution of ethyl 4-[3-(2-{3-cyano-4-[(1-methylethyl)oxy]phenyl}-1,3-thiazol-5-yl)-2-ethylphenyl]butanoate (D121) (320 mg) in isopropanol (8 mL) and water (4.00 mL) at room temperature was added 2M sodium hydroxide (1.729 mL). The reaction mixture was stirred at room temperature overnight. After concentration, the residue was dissolved in water and acidified with 2N HCl to pH=5. The solvent was removed in vacuo, the residue was dissolved in THF, and filtered. The filtrate was purified by Mass Directed AutoPrep to give 4-[3-(2-{3-cyano-4-[(1-methylethyl)oxy]phenyl}-1,3-thiazol-5-yl)-2-ethylphenyl]butanoic acid (E50) (35 mg). δH (DMSO-d₆, 400 MHz): 1.05 (3H, t), 1.35 (6H, m), 1.78 (2H, m), 2.29 (2H, t), 2.68 (4H, m), 4.90 (1H, m), 7.22 (2H, m), 7.28 (1H, m), 7.43 (1H, d), 7.85 (1H, s), 8.21 (1H, dd), 8.26 (1H, d). MS (ES): C₂₅H₂₆N₂O₃S requires 434; found 435.2 (M+H⁺).

### EXAMPLE-51

### 4-[3-(2-{3-cyano-4-[(1-methylethyl)oxy]phenyl}-1,3-thiazol-5-yl)-5-fluoro-2-(methyloxy)phenyl]butanoic acid (E51)

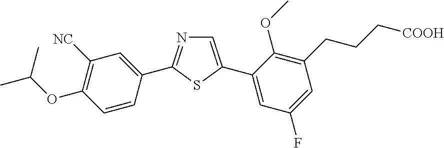

To a solution of ethyl 4-[3-(2-{3-cyano-4-[(1-methylethyl)oxy]phenyl}-1,3-thiazol-5-yl)-5-fluoro-2-(methyloxy)phenyl]butanoate (D122) (280 mg) in isopropanol (2 mL) and water (0.750 mL) at room temperature was added 2M sodium hydroxide (0.899 mL). The reaction mixture was stirred at room temperature for 2 h. After concentration, the residue was dissolved in water and acidified with 1N HCl to pH=5. The solvent was removed in vacuo, the residue was dissolved in THF, and filtered. The filtrate was purified by Mass Directed AutoPrep to give 4-[3-(2-{3-cyano-4-[(1-methylethyl)oxy]phenyl}-1,3-thiazol-5-yl)-5-fluoro-2-(methyloxy)phenyl]butanoic acid (E51) (18 mg). δH (DMSO-d₆, 400 MHz): 1.36 (6H, d), 1.85 (2H, m), 2.07 (1H, s), 2.30 (2H, t), 2.69 (2H, t), 3.64 (3H, s), 4.90 (1H, m), 7.14 (1H, dd), 7.43 (1H, d), 7.65 (1H, dd), 8.23 (1H, dd), 8.30 (1H, d), 8.46 (1H, s). SF (DMSO-d₆, 376 MHz): −117.9. MS (ES): C₂₄H₂₃FN₂O₄S requires 454; found 455.1 (M+H⁺).

### EXAMPLE-52

### 4-[3-(2-{3-chloro-4-[(1-methylethyl)oxy]phenyl}-1,3-thiazol-5-yl)-2-ethylphenyl]butanoic acid (E52)

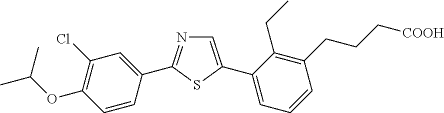

To a solution of ethyl 4-[3-(2-{3-chloro-4-[(1-methylethyl)oxy]phenyl}-1,3-thiazol-5-yl)-2-ethylphenyl]butanoate (D125) (113 mg) in isopropanol (15 mL) and water (5 mL) at room temperature was added 2M sodium hydroxide (0.239 mL). The reaction mixture was stirred at room temperature overnight. After concentration, the residue was dissolved in water and acidified with 1N HCl to pH=5. The solvent was removed in vacuo, the residue was dissolved in THF, and filtered. The filtrate was purified by Mass Directed AutoPrep to give 4-[3-(2-{3-chloro-4-[(1-methylethyl)oxy]phenyl}-1,3-thiazol-5-yl)-2-ethylphenyl]butanoic acid (E52) (30 mg). δH (DMSO-d₆, 400 MHz): 1.05 (3H, t), 1.34 (6H, d), 1.79 (2H, m), 2.33 (2H, t), 2.69 (4H, m), 4.79 (1H, m), 7.22 (2H, m), 7.29 (2H, m), 7.82 (1H, s), 7.87 (1H, dd), 7.98 (1H, d), 12.12 (1H, br s). MS (ES): C₂₄H₂₆ClNO₃S requires 443; found 444.0 (M+H⁺).

### EXAMPLE-53

### N-{2-[3-(2-{3-cyano-4-[(1-methylethyl)oxy]phenyl}-1,3-thiazol-5-yl)-2-ethylphenyl]ethyl}-N-methylglycine (E53)

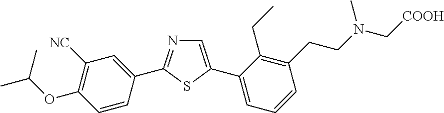

To a solution of methyl 1-{2-[3-(2-{3-cyano-4-[(1-methylethyl)oxy]phenyl}-1,3-thiazol-5-yl)-2-ethylphenyl]ethyl}-3-azetidinecarboxylate (D132) (183 mg) in isopropanol (40 mL) and water (10 mL) stirred under nitrogen at room temperature was added a solution of NaOH (30.7 mg) in water in one charge. The reaction mixture was stirred at room temperature overnight. Isopropanol was removed in vacuo. The residue was dissolved in water and acidified with 1N HCl to pH=5. The solvent was removed in vacuo, the residue was dissolved in THF, and filtered. The filtrate was purified by Mass Directed AutoPrep to give N-{2-[3-(2-{3-cyano-4-[(1-methylethyl)oxy]phenyl}-1,3-thiazol-5-yl)-2-ethylphenyl]ethyl}-N-methylglycine (E53) (22 mg) as a white solid. δH (DMSO-d₆, 400 MHz): 1.10 (3H, t), 1.36 (6H, d), 2.71 (2H, m), 2.95 (3H, s), 3.13 (2H, m), 3.30 (2H, m), 4.18 (2H, s), 4.91 (1H, m), 7.33 (3H, m), 7.44 (1H, d), 7.85 (1H, s), 8.21 (1H, d), 8.28 (1H, d), 9.95 (1H, br s). δF (DMSO-d₆, 376 MHz): −73.6. MS (ES): C₂₆H₂₉N₃O₃S requires 463; found 464.2 (M+H⁺).

### EXAMPLE-54

### N-{[3-(2-{3-chloro-4-[(1-methylethyl)oxy]phenyl}-1,3-thiazol-5-yl)-2-ethylphenyl]methyl}-N-methylglycine (E54)

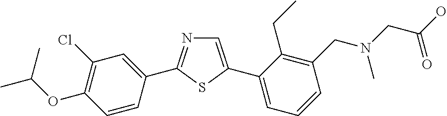

To a solution of methyl N-{[3-(2-{3-chloro-4-[(1-methylethyl)oxy]phenyl}-1,3-thiazol-5-yl)-2-ethylphenyl]methyl}-N-methylglycinate (D133) (160 mg) in isopropanol (40 mL) and water (10 mL) stirred under nitrogen at room temperature was added a solution of NaOH (27.1 mg) in water in one charge. The reaction mixture was stirred at room temperature overnight. Isopropanol was removed in vacuo. The residue was dissolved in water and acidified with 1N HCl to pH=5. The solvent was removed in vacuo, the residue was dissolved in THF, and filtered. The filtrate was purified by Mass Directed AutoPrep to give N-{[3-(2-{3-chloro-4-[(1-methylethyl)oxy]phenyl}-1,3-thiazol-5-yl)-2-ethylphenyl]methyl}-N-methylglycine (E54) (48 mg) as a white solid. δH (DMSO-d₆, 400 MHz): 1.03 (3H, t), 1.34 (6H, d), 2.77 (3H, s), 2.86 (2H, m), 4.17 (2H, s), 4.43 (2H, s), 4.80 (1H, m), 7.32 (1H, d), 7.41 (1H, t), 7.50 (1H, d), 7.62 (1H, d), 7.87 (2H, m), 7.99 (1H, d), 9.99 (1H, br s). δF (DMSO-d₆, 376 MHz): −74.1. MS (ES): C₂₄H₂₇ClN₂O₃S requires 458; found 459.0 (M+H⁺).

### EXAMPLE-55

### N-{[3-(2-{3-cyano-4-[(1-methylethyl)oxy]phenyl}-1,3-thiazol-5-yl)-2-ethylphenyl]methyl}-N-methylglycine (E55)

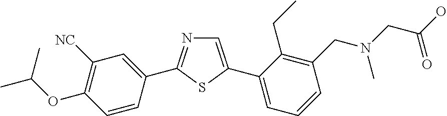

To a solution of methyl N-{[3-(2-{3-cyano-4-[(1-methylethyl)oxy]phenyl}-1,3-thiazol-5-yl)-2-ethylphenyl]methyl}-N-methylglycinate (D134) (140 mg) in isopropanol (40 mL) and water (10 mL) stirred under nitrogen at room temperature was added a solution of NaOH (24.2 mg) in water in one charge. The reaction mixture was stirred at room temperature overnight. Isopropanol was removed in vacuo. The residue was dissolved in water and acidified with 1N HCl to pH=5. The solvent was removed in vacuo, the residue was dissolved in THF, and filtered. The filtrate was purified by Mass Directed AutoPrep to give N-{[3-(2-{3-cyano-4-[(1-methylethyl)oxy]phenyl}-1,3-thiazol-5-yl)-2-ethylphenyl]methyl}-N-methylglycine (E55) (58 mg) as a white solid. δH (DMSO-d₆, 400 MHz): 1.03 (3H, t), 1.36 (6H, d), 2.76 (3H, s), 2.87 (2H, m), 4.15 (2H, s), 4.41 (2H, s), 4.91 (1H, m), 7.45 (3H, m), 7.62 (1H, d), 7.89 (1H, s), 8.22 (1H, dd), 8.28 (1H, d), 10.11 (1H, br s), 14.19 (1H, br s). δF (DMSO-d₆, 376 MHz): −74.0. MS (ES): C₂₅H₂₇N₃O₃S requires 449; found 450.2 (M+H⁺).

### EXAMPLE-56

### N-{2-[3-(2-{3-chloro-4-[(1-methylethyl)oxy]phenyl}-1,3-thiazol-5-yl)-2-ethylphenyl]ethyl}-N-methylglycine (E56)

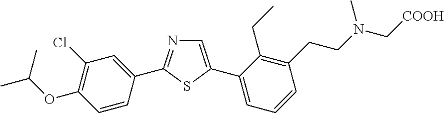

To a solution of methyl N-{2-[3-(2-{3-chloro-4-[(1-methylethyl)oxy]phenyl}-1,3-thiazol-5-yl)-2-ethylphenyl]ethyl}-N-methylglycinate (D135) (120 mg) in isopropanol (40 mL) and water (10 mL) stirred under nitrogen at room temperature was added a solution of NaOH (19.7 mg) in water in one charge. The reaction mixture was stirred at room temperature overnight. Isopropanol was removed in vacuo. The residue was dissolved in water and acidified with 1N HCl to pH=5. The solvent was removed in vacuo, the residue was dissolved in THF, and filtered. The filtrate was purified by Mass Directed AutoPrep to give N-{2-[3-(2-{3-chloro-4-[(1-methylethyl)oxy]phenyl}-1,3-thiazol-5-yl)-2-ethylphenyl]ethyl}-N-methylglycine (E56) (29 mg) as a white solid. δH (DMSO-d₆, 400 MHz): 1.09 (3H, t), 1.34 (6H, d), 2.73 (2H, d), 2.96 (3H, s), 3.13 (2H, m), 3.30 (2H, m), 4.19 (2H, s), 4.79 (1H, m), 7.31 (4H, m), 7.82 (1H, s), 7.87 (1H, dd), 7.99 (1H, d), 10.02 (1H, br s), 14.13 (1H, br s). δF (DMSO-d₆, 376 MHz): −73.7. MS (ES): C₂₆H₂₉ClN₂O₃S requires 472; found 473.0 (M+H⁺).

### EXAMPLE-57

### N-{[3-(2-{3-cyano-4-[(1-methylethyl)oxy]phenyl}-1,3-thiazol-5-O-2-ethylphenyl]methyl}-N-methyl-β-alanine (E57)

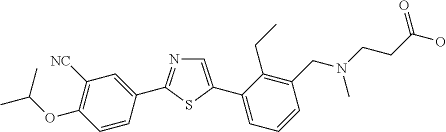

A solution of 5-[5-(2-ethyl-3-formylphenyl)-1,3-thiazol-2-yl]-2-[(1-methylethyl)oxy]benzonitrile (D126) (200 mg), ethyl beta-alaninate (163 mg) and acetic acid (0.1 mL) in dichloromethane (DCM) (10 mL) was stirred at room temperature for 15 min, then sodium triacetoxyborohydride (338 mg) was added. The mixture was stirred at room temperature for 2 h, then formaldehyde (0.146 mL) was added. The mixture was stirred at room temperature overnight. After concentration, the residue was partitioned between ethyl acetate and water, the organic phase was washed with saturated brine and concentrated, the residue was dissolved in DMF (5 mL) and aqueous 2M NaOH (2 mL) was added. The mixture was stirred at 50° C. for 1 h. After cooling the reaction, the reaction mixture was acidified with aqueous 2M HCl to Ph=5-6, extracted with ethyl acetate for two times. The combined organic phases were concentrated and the residue was purified by mass Directed AutoPrep to afford N-{[3-(2-{3-cyano-4-[(1-methylethyl)oxy]phenyl}-1,3-thiazol-5-yl)-2-ethylphenyl]methyl}-N-methyl-β-alanine (E57) (155 mg). δH (DMSO-d₆, 400 MHz): 1.05 (3H, t), 1.36 (6H, d), 2.76 (3H, s), 2.82 (4H, m), 3.41 (2H, s), 4.44 (2H, s), 4.91 (1H, m), 7.46 (3H, m), 7.63 (1H, dd), 7.88 (1H, s), 8.22 (1H, dd), 8.28 (1H, d), 9.36 (1H, br s). δF (DMSO-d₆, 376 MHz): −73.8. MS (ES): C₂₆H₂₉N₃O₃S requires 463; found 464.2 (M+H⁺).

### EXAMPLE-58

### 4-[4-(2-{3-cyano-4-[(1-methylethyl)oxy]phenyl}-1,3-thiazol-5-yl)-3-ethyl-2-pyridinyl]butanoic acid (E58)

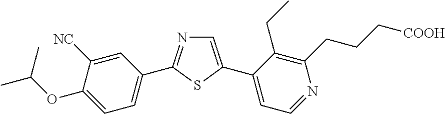

To a suspension of 5-[5-(2-chloro-3-ethyl-4-pyridinyl)-1,3-thiazol-2-yl]-2-[(1-methylethyl)oxy]benzonitrile (D136) (200 mg), tris(1,1-dimethylethyl)phosphane (30.2 mg), Pd₂(dba)₃ (95 mg) and Cs₂CO₃ (67.9 mg) in tetrahydrofuran (THF) (5 mL) was added 0.5M bromo[4-(ethyloxy)-4-oxobutyl]zinc (5 mL) in THF. The reaction was stirred at 60° C. under nitrogen for 1 h. After that, the reaction was quenched with aqueous ammonium chloride, partitioned between ethyl acetate and water. The organic phase was washed with water and saturated brine. After removing solvent, the residue was dissolved in DMF (5 mL) and aqueous 2M NaOH (2 mL) was added. The mixture was stirred at 50° C. for 1 h. After cooling the reaction, the mixture was partitioned between ethyl acetate and water. The aqueous phase was separated and colleceted, acidified with aqueous 2M HCl to Ph=4-5, extracted with ethyl acetate. The combined organic phase was evaporated to give the crude product, which was purified by Mass Directed AutoPrep to afford 4-[4-(2-{3-cyano-4-[(1-methylethyl)oxy]phenyl}-1,3-thiazol-5-yl)-3-ethyl-2-pyridinyl]butanoic acid (E58) (40 mg). δH (DMSO-d₆, 400 MHz): 1.14 (3H, t), 1.33 (6H, d), 1.94 (2H, m), 2.39 (2H, t), 2.88 (2H, m), 2.98 (2H, t), 4.92 (1H, m), 7.46 (1H, d), 7.67 (1H, d), 8.21 (1H, s), 8.26 (1H, dd), 8.35 (1H, d), 8.59 (1H, d). δF (DMSO-d₆, 376 MHz): −74.5. MS (ES): C₂₄H₂₅N₃O₃S requires 435; found 436.2 (M+H⁺).

### EXAMPLE-59

### N-{2-[3-(2-{3-cyano-4-[(1-methylethyl)oxy]phenyl}-1,3-thiazol-5-yl)-2-ethylphenyl]ethyl}-N-methyl-b-alanine (E59)

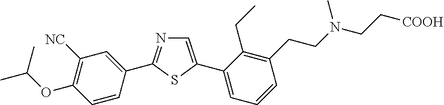

A solution of 5-{5-[2-ethyl-3-(2-oxoethyl)phenyl]-1,3-thiazol-2-yl}-2-[(1-methylethyl)oxy]benzonitrile (D129) (100 mg), ethyl beta-alaninate (79 mg) and acetic acid (0.1 mL) in dichloromethane (DCM) (10 mL) was stirred at room temperature for 15 min, then sodium triacetoxyborohydride (163 mg) was added. The mixture was stirred at room temperature for 2 h, then formaldehyde (0.071 mL) was added. The mixture was stirred at room temperature overnight. After concentration, the residue was partitioned between ethyl acetate and water, washed with saturated brine. The organic phase was collected and evaporated, the residue was dissolved in DMF (5 mL) and 2M NaOH (2 mL) solution was added. The mixture was stirred at 50° C. for 1 h, then cooled to room temperature. This reaction mixture was acidified with 2M HCl to pH 5-6, extracted with ethyl acetate for three times. The combined organic phase was concentrated and the residue purified by Mass Directed AutoPrep to afford N-{2-[3-(2-{3-cyano-4-[(1-methylethyl)oxy]phenyl}-1,3-thiazol-5-yl)-2-ethylphenyl]ethyl}-N-methyl-β-alanine (E59) (145 mg). δH (DMSO-d₆, 400 MHz): 1.10 (3H, t), 1.36 (6H, d), 2.72 (2H, m), 2.81 (2H, m), 2.90 (3H, s), 3.44 (4H, m), 4.90 (1H, m), 7.31 (2H, m), 7.38 (1H, m), 7.44 (1H, d), 7.85 (1H, s), 8.21 (1H, dd), 8.27 (1H, d), 9.68 (1H, br s), 12.82 (1H, br s). δF (DMSO-d₆, 376 MHz): −73.7. MS (ES): C₂₇H₃₁N₃O₃S requires 477; found 478.2 (M+H⁺).

### EXAMPLE-60

### 3-[3-(5-{3-chloro-4-[(1-methylethyl)oxy]phenyl}-1,2,4-oxadiazol-3-yl)phenyl]propanoic acid (E60)

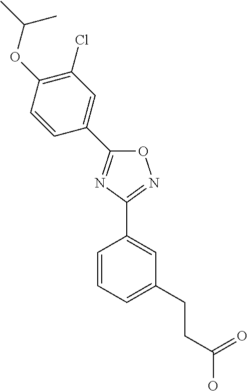

TFA (1 mL, 12.98 mmol) was added dropwise to an ice cooled solution of 1,1-dimethylethyl 3-[3-(5-{3-chloro-4-[(1-methylethyl)oxy]phenyl}-1,2,4-oxadiazol-3-yl)phenyl]propanoate (D140; 1.93 g, 4.36 mmol) in dichloromethane (DCM) (40 mL), and the reaction mixture was stirred for an hour. The reaction mixture was evaporated to yield a light yellow solid which was triturated under diethyl ether to give the title compound as a white coloured solid (1.2 g)

MS: (+ve ion electrospray) m/z 387 [MH⁺];

¹H NMR (400 MHz, CDCl₃) δ ppm 8.25 (d, J=2.0 Hz, 1H), 8.06 (dd, J=8.5, 2.0 Hz, 1H), 8.02 (s, 1H), 7.99-8.03 (m, 1H), 7.44 (t, J=8.0 Hz, 1H), 7.38 (d, J=7.5 Hz, 1H), 7.06 (d, J=8.5 Hz, 1H), 4.72 (spt, J=6.0 Hz, 1H), 3.06 (t, J=8.0 Hz, 2H), 2.77 (t, J=8.0 Hz, 2H), 1.45 (d, J=6.0 Hz, 6H)

S1P1 Tango assay—96 Well Format

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were suspended in assay medium (Invitrogen Freestyle Expression Medium) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (SIP) (row 1, column 1-10) in a Corning black-well, clear bottom 96-well plate. Cells were incubated at 37° C., 5% CO₂ for 44-48 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free control wells.

After incubation at 37° C., 5% CO₂ for 5 h, 25 μl of 6× substrate mixture (6 μl Solution A (1 mg LiveBLAzer™-FRET B/G substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted Green emission values. The dose response curve is based on sigmoidal dose-response model. All ratio data was normalized based upon the maximum emission ratio of positive control (SIP) and minimum emission ratio of negative control (DMSO) on each plate. The intrinsic activity (IA) of each compound would be the normalized percentage of its maximum response after curve fitting.

Examples 1 to 27 had a pEC50≧6.0 in this assay Except for Examples 3, 4 and 21 with a pEC50<6.

S1P1 Tango assay—384 Well Format

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml. All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat #K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Examples 20, 22, 23, 54 and 58 had a pEC50≧6 in this assay. Examples 25, 33, 57, 55 and 57 had a pEC50≧7.0 in this assay. Examples 7, 8, 14, 24, 32, 34, 41, 36, 37, 38, 40, 41, 144, 145, 146, 48, 50, 51, 52, 53, 56 had a pEC50≧8 in this assay.

Examples 11, 12, 15, 17, 28, 29, 30, 31, 35, 39, 49 and 59 had a pEC50≧9 in this assay.

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/ml penicillin, 100 μg/ml streptomycin) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (SIP) (row 1, column 1-10) in a Corning black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (1 mg LiveBLAzer™-FRET B/G Substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (SIP) and minimum emission ratio of negative control (DMSO) on each plate. The intrinsic activity (IA) of each compound would be the normalized percentage of its maximum response after curve fitting.

### S1P3 GeneBlazer Assay—384 Well Format

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were harvested from growth medium and resuspended in thawing medium (90% DMEM, 10% Charcoal-stripped FBS, 0.1 mM NEAA, 25 mM HEPES (pH 7.3), 100 U/ml penicillin, 100 μg/ml streptomycin) at a density of ˜200,000 cells/ml.

All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P3 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of thawing medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat #K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Exemplified compounds of the invention tested in at least one of the above assays had a pEC50≦5.5

1. A compound of formula (I) or a salt thereof: 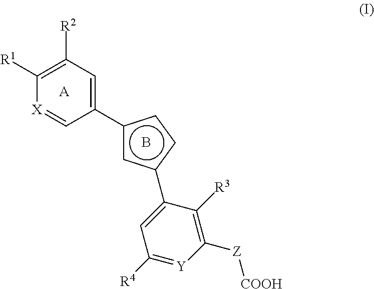
wherein
X is CH or N;
Y is CR⁵ or N;
Z is C₍₀₋₅₎alkyl optionally interrupted by O, S or N and optionally substituted by C₍₁₋₃₎alkyl or OH;
B is a 5-membered heteroaryl ring selected from: 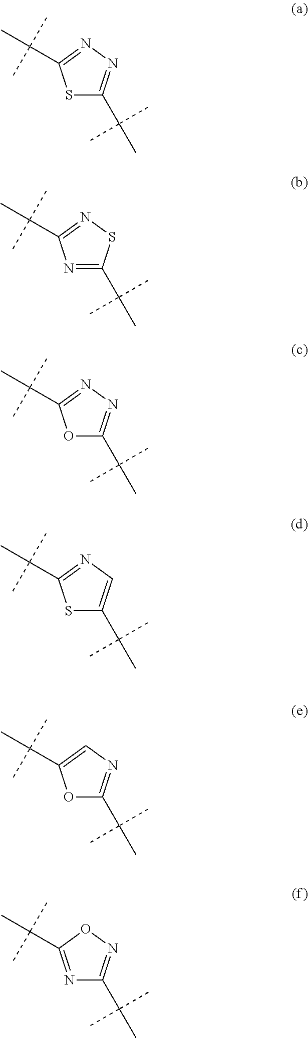
R¹ is C₍₁₋₄₎alkoxy;
R² is cyano or chloro;
R³ is hydrogen, halogen, trifluoromethyl, C₍₁₋₅₎alkyl, C₍₁₋₅₎alkoxy or CN;
R⁴ is hydrogen, halogen, trifluoromethyl, C₍₁₋₃₎alkyl or C₍₁₋₃₎alkoxy; and
R⁵ is hydrogen, halogen, trifluoromethyl, C₍₁₋₃₎alkyl or C₍₁₋₃₎alkoxy, with the proviso that the compound is not 3-(3-(5-(3-cyano-4-isopropoxyphenyl)-1,2,4-oxadiazol-3-yl)-5-methylphenyl)propanoic acid.

2. A compound according to claim 1 or a salt thereof, wherein:
Y is CR⁵ or N;
Z is C₍₀₋₅₎alkyl optionally interrupted by O or N and optionally substituted by C₍₁₋₃₎alkyl;
B is (a), (b), (c), (d) or (f);
R¹ is C₍₁₋₄₎alkoxy;
R² is cyano or chloro;
R³ is hydrogen, methyl, ethyl, propyl, methoxy, chloro or trifluoromethyl;
R⁴ is hydrogen or fluoro; and
R⁵ is hydrogen.

3. A compound selected from the group consisting of:
3-[3-(5-{3-chloro-4-[(1-methylethyl)oxy]phenyl}-1,2,4-oxadiazol-3-yl)-2-methylphenyl]propanoic acid;
4-[3-(5-{3-chloro-4-[(1-methylethyl)oxy]phenyl}-1,2,4-oxadiazol-3-yl)-2-methylphenyl]butanoic acid;
(2R)-3-[3-(5-{5-chloro-6-[(1-methylethyl)oxy]-3-pyridinyl}-1,2,4-oxadiazol-3-yl)-2-methylphenyl]-2-methylpropanoic acid;
4-[3-(5-{5-chloro-6-[(1-methylethyl)oxy]-3-pyridinyl}-1,2,4-oxadiazol-3-yl)-2-methylphenyl]butanoic acid;
3-[3-(5-{3-cyano-4-[(1-methylethyl)oxy]phenyl}-1,2,4-oxadiazol-3-yl)-2-methylphenyl]propanoic acid;
4-[3-(5-{3-cyano-4-[(1-methylethyl)oxy]phenyl}-1,2,4-oxadiazol-3-yl)-2-methylphenyl]butanoic acid;
4-[4-(5-{3-chloro-4-[(1-methylethyl)oxy]phenyl}-1,2,4-oxadiazol-3-yl)-3-methyl-2-pyridinyl]butanoic acid;
3-[2-chloro-3-(5-{5-chloro-6-[(1-methylethyl)oxy]-3-pyridinyl}-1,2,4-oxadiazol-3-yl)phenyl]propanoic acid;
4-[2-chloro-3-(5-{5-chloro-6-[(1-methylethyl)oxy]-3-pyridinyl}-1,2,4-oxadiazol-3-yl)phenyl]butanoic acid;
3-[2-chloro-3-(5-{3-cyano-4-[(1-methylethyl)oxy]phenyl}-1,2,4-oxadiazol-3-yl)phenyl]propanoic acid;
4-[2-chloro-3-(5-{3-cyano-4-[(1-methylethyl)oxy]phenyl}-1,2,4-oxadiazol-3-yl)phenyl]butanoic acid;
6-[2-chloro-3-(5-{3-cyano-4-[(1-methylethyl)oxy]phenyl}-1,2,4-oxadiazol-3-yl)phenyl]hexanoic acid;
3-[3-(5-{5-chloro-6-[(1-methylethyl)oxy]-3-pyridinyl}-1,2,4-oxadiazol-3-yl)-2-ethylphenyl]propanoic acid;
4-[3-(5-{5-chloro-6-[(1-methylethyl)oxy]-3-pyridinyl}-1,2,4-oxadiazol-3-yl)-2-ethylphenyl]butanoic acid;
4-[3-(5-{3-cyano-4-[(1-methylethyl)oxy]phenyl}-1,2,4-oxadiazol-3-yl)-2-ethylphenyl]butanoic acid;
3-[3-(5-{3-chloro-4-[(1-methylethyl)oxy]phenyl}-1,2,4-oxadiazol-3-yl)-2-(methyloxy)phenyl]propanoic acid;
4-[3-(5-{3-chloro-4-[(1-methylethyl)oxy]phenyl}-1,2,4-oxadiazol-3-yl)-2-(methyloxy)phenyl]butanoic acid;
3-[3-(5-{3-cyano-4-[(1-methylethyl)oxy]phenyl}-1,2,4-oxadiazol-3-yl)-2-(methyloxy)phenyl]propanoic acid;
4-[3-(5-{3-cyano-4-[(1-methylethyl)oxy]phenyl}-1,2,4-oxadiazol-3-yl)-2-(methyloxy)phenyl]butanoic acid;
3-[4-(5-{3-cyano-4-[(1-methylethyl)oxy]phenyl}-1,2,4-oxadiazol-3-yl)-2-pyridinyl]propanoic acid;
4-[4-(5-{3-cyano-4-[(1-methylethyl)oxy]phenyl}-1,2,4-oxadiazol-3-yl)-2-pyridinyl]butanoic acid;
3-[5-(5-{3-cyano-4-[(1-methylethyl)oxy]phenyl}-1,2,4-oxadiazol-3-yl)-2-fluorophenyl]propanoic acid;
4-[5-(5-{3-cyano-4-[(1-methylethyl)oxy]phenyl}-1,2,4-oxadiazol-3-yl)-2-fluorophenyl]butanoic acid;
3-[3-(5-{3-cyano-4-[(1-methylethyl)oxy]phenyl}-1,2,4-oxadiazol-3-yl)-2-(trifluoromethyl)phenyl]propanoic acid;
4-[3-(5-{3-cyano-4-[(1-methylethyl)oxy]phenyl}-1,2,4-oxadiazol-3-yl)-2-(trifluoromethyl)phenyl]butanoic acid;
3-(5-{3-cyano-4-[(1-methylethyl)oxy]phenyl}-1,2,4-oxadiazol-3-yl)-2-methylbenzoic acid;
{[3-(5-{5-chloro-6-[(1-methylethyl)oxy]-3-pyridinyl}-1,2,4-oxadiazol-3-yl)-2-methylphenyl]oxy}acetic acid;
4-[3-(5-{3-chloro-4-[(1-methylethyl)oxy]phenyl}-1,2,4-oxadiazol-3-yl)-2-ethylphenyl]butanoic acid;
3-[3-(5-{3-chloro-4-[(1-methylethyl)oxy]phenyl}-1,2,4-oxadiazol-3-yl)-2-ethylphenyl]propanoic acid;
3-[3-(5-{3-cyano-4-[(1-methylethyl)oxy]phenyl}-1,2,4-oxadiazol-3-yl)-5-fluoro-2-(methyloxy)phenyl]propanoic acid;
4-[3-(5-{3-cyano-4-[(1-methylethyl)oxy]phenyl}-1,2,4-oxadiazol-3-yl)-5-fluoro-2-(methyloxy)phenyl]butanoic acid;
4-[3-(5-{5-chloro-6-[(1-methylethyl)oxy]-3-pyridinyl}-1,2,4-oxadiazol-3-yl)-5-fluoro-2-(methyloxy)phenyl]butanoic acid;
3-[3-(5-{5-chloro-6-[(1-methylethyl)oxy]-3-pyridinyl}-1,2,4-oxadiazol-3-yl)-5-fluoro-2-(methyloxy)phenyl]propanoic acid;
3-[3-(5-{3-chloro-4-[(1-methylethyl)oxy]phenyl}-1,2,4-oxadiazol-3-yl)-5-fluoro-2-(methyloxy)phenyl]propanoic acid;
4-[3-(5-{3-chloro-4-[(1-methylethyl)oxy]phenyl}-1,2,4-oxadiazol-3-yl)-5-fluoro-2-(methyloxy)phenyl]butanoic acid;
4-[3-(5-{3-chloro-4-[(1-methylethyl)oxy]phenyl}-1,2,4-oxadiazol-3-yl)-2-propylphenyl]butanoic acid;
4-[3-(5-{3-cyano-4-[(1-methylethyl)oxy]phenyl}-1,2,4-oxadiazol-3-yl)-2-propylphenyl]butanoic acid;
4-{[3-(5-{3-cyano-4-[(1-methylethyl)oxy]phenyl}-1,2,4-oxadiazol-3-yl)-2-ethylphenyl]oxy}butanoic acid;
4-{[3-(5-{3-chloro-4-[(1-methylethyl)oxy]phenyl}-1,2,4-oxadiazol-3-yl)-2-ethylphenyl]oxy}butanoic acid;
{[3-(5-{3-cyano-4-[(1-methylethyl)oxy]phenyl}-1,2,4-oxadiazol-3-yl)-2-ethylphenyl]oxy}acetic acid;
4-[3-(5-{3-cyano-4-[(1-methylethyl)oxy]phenyl}-1,3,4-oxadiazol-2-yl)-2-ethylphenyl]butanoic acid;
4-[3-(5-{3-cyano-4-[(1-methylethyl)oxy]phenyl}-1,3,4-thiadiazol-2-yl)-2-ethylphenyl]butanoic acid;
N-{[3-(5-{3-chloro-4-[(1-methylethyl)oxy]phenyl}-1,3,4-thiadiazol-2-yl)-2-ethylphenyl]methyl}-N-methylglycine;
4-[3-(3-{3-cyano-4-[(1-methylethyl)oxy]phenyl}-1,2,4-thiadiazol-5-yl)-2-ethylphenyl]butanoic acid;
4-[3-(5-{3-chloro-4-[(1-methylethyl)oxy]phenyl}-1,2,4-thiadiazol-3-yl)-2-ethylphenyl]butanoic acid;
4-[3-(5-{3-cyano-4-[(1-methylethyl)oxy]phenyl}-1,2,4-thiadiazol-3-yl)-2-ethylphenyl]butanoic acid;
N-{[3-(5-{3-cyano-4-[(1-methylethyl)oxy]phenyl}-1,2,4-thiadiazol-3-yl)-2-ethylphenyl]methyl}-N-methylglycine;
N-{[3-(5-{3-cyano-4-[(1-methylethyl)oxy]phenyl}-1,2,4-thiadiazol-3-yl)-2-ethylphenyl]methyl}-b-alanine;
N-{2-[3-(5-{3-chloro-4-[(1-methylethyl)oxy]phenyl}-1,2,4-thiadiazol-3-yl)-2-ethylphenyl]ethyl}-N-methylglycine;
4-[3-(2-{3-cyano-4-[(1-methylethyl)oxy]phenyl}-1,3-thiazol-5-yl)-2-ethylphenyl]butanoic acid;
4-[3-(2-{3-cyano-4-[(1-methylethyl)oxy]phenyl}-1,3-thiazol-5-yl)-5-fluoro-2-(methyloxy)phenyl]butanoic acid;
4-[3-(2-{3-chloro-4-[(1-methylethyl)oxy]phenyl}-1,3-thiazol-5-yl)-2-ethylphenyl]butanoic acid;
N-{2-[3-(2-{3-cyano-4-[(1-methylethyl)oxy]phenyl}-1,3-thiazol-5-yl)-2-ethylphenyl]ethyl}-N-methylglycine;
N-{[3-(2-{3-chloro-4-[(1-methylethyl)oxy]phenyl}-1,3-thiazol-5-yl)-2-ethylphenyl]methyl}-N-methylglycine;
N-{[3-(2-{3-cyano-4-[(1-methylethyl)oxy]phenyl}-1,3-thiazol-5-yl)-2-ethylphenyl]methyl}-N-methylglycine;
N-{2-[3-(2-{3-chloro-4-[(1-methylethyl)oxy]phenyl}-1,3-thiazol-5-yl)-2-ethylphenyl]ethyl}-N-methylglycine;
N-{[3-(2-{3-cyano-4-[(1-methylethyl)oxy]phenyl}-1,3-thiazol-5-yl)-2-ethylphenyl]methyl}-N-methyl-β-alanine;
4-[4-(2-{3-cyano-4-[(1-methylethyl)oxy]phenyl}-1,3-thiazol-5-yl)-3-ethyl-2-pyridinyl]butanoic acid;
N-{2-[3-(2-{3-cyano-4-[(1-methylethyl)oxy]phenyl}-1,3-thiazol-5-yl)-2-ethylphenyl]ethyl}-N-methyl-b-alanine; and
3-[3-(5-{3-chloro-4-[(1-methylethyl)oxy]phenyl}-1,2,4-oxadiazol-3-yl)phenyl]propanoic acid,
and salts thereof.

4. A method of treating a subject with a condition or disorder mediated by a S1P1 receptor comprising administering to the subject with said condition or disorder a therapeutically effective amount of a compound of formula (I) or a pharmaceutically acceptable salt thereof according to claim 1.

5. A method according to claim 4, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

6. A method according to claim 5, wherein the condition is multiple sclerosis.

7. (canceled)

8. (canceled)

9. (canceled)

10. A pharmaceutical composition comprising a compound of formula (I) according claim 1 or a pharmaceutically acceptable salt thereof.

11. (canceled)

12. (canceled)
